SEQUENCE LISTING (2 sequences)

SEQ ID NO: 1 - protein, 5 aa, Artificial Sequence
  Description of Artificial Sequence Synthetic peptide
DEADH

SEQ ID NO: 2 - protein, 4 aa, Artificial Sequence
  Description of Artificial Sequence Synthetic peptide
KDEL

(19) United States
(12) Patent Application Publication
(10) Pub. No.: US 2007/0134657 A1
(43) Pub. Date: Jun. 14, 2007

(54) Methods and compositions relating to gradient exposed cells

(75) Inventors: Mark C. Poznansky, Charlestown, MA (US); Rachel Rutishauer, New Haven, CT (US)

Correspondence Address: EDWARDS ANGELL PALMER & DODGE LLP, P.O. BOX 55874, BOSTON, MA, 02205, US

(73) Assignee: GENERAL HOSPITAL CORPORATION, THE, BOSTON, MA (US)

(21) Appl. No.: 10/537,610

(22) PCT Filed: Dec. 8, 2003
(86) PCT No.: PCT/US03/38958

§ 371 Date: Dec. 18, 2006

Related U.S. Application Data
(60) Provisional application No. 60/431,424, filed on Dec. 6, 2002.
(60) Provisional application No. 60/438,848, filed on Jan. 9, 2003.
(60) Provisional application No. 60/445,049, filed on Feb. 5, 2003.

(51) Int. Cl. C12Q 1/68; C12N 15/09

(52) U.S. Cl. 435/6; 435/455

(57) ABSTRACT

The invention relates to methods and compositions for stimulating or inhibiting directed cell migration.

### CROSS-REFERENCE TO RELATED APPLICATIONS

This application claims priority to U.S. Application Ser. No. 60/431,424 filed Dec. 6, 2003, U.S. Application Ser. No. 60/438,848 filed Jan. 9, 2003, and U.S. Application Ser. No. 60/445,049 filed Feb. 5, 2003.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the numbered paragraphs, or in the text itself, and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by NIH grants R21 A145898-01. The government may have certain rights to the invention.

### FIELD OF THE INVENTION

The invention is directed to methods and compositions relating to modulation of gene expression in cells in chemotactic and fugetactic gradients.

### BACKGROUND OF THE INVENTION

Cell movement in response to specific stimuli occurs in prokaryotes and eukaryotes (Doetsch R N and Seymour W F., 1970; Bailey G B et al., 1985). Cell movement by these organisms has been classified into three types; chemotaxis, which is cell movement along a gradient towards an increasing concentration of an agent (e.g., a chemical); negative chemotaxis, which is cell movement towards a decreasing concentration of an agent, and chemokinesis, which is the random movement of cells.

The receptors and signal transduction pathways affected by the actions of specific chemotactically active compounds have been extensively defined in prokaryotic cells. Study of E. coli chemotaxis has revealed that a chemical which attracts the bacteria at some concentrations and conditions may also act as a repellant at others (i.e., a “negative chemotactic chemical” or “chemorepellent”) (Tsang N et al., 1973; Repaske D and Adler J. 1981; Tisa L S and Adler J., 1995; Taylor B L and Johnson M S., 1998).

Chemotaxis and chemokinesis have been observed to occur in mammalian cells (McCutcheon M W, Wartman W and H M Dixon, 1934; Lotz M and H Harris; 1956; Boyden S V 1962) in response to the class of proteins, called chemokines (Ward S G and Westwick J; 1998; Kim C H et al., 1998; Baggiolini M, 1998; Farber J M; 1997). Chemokines induce cell motion by signaling through G-protein coupled receptors (Wells T N et al., 1998).

G-protein coupled receptors include a wide range of biologically active receptors, such as hormone, viral, growth factor and neuroreceptors. The G-protein family of coupled receptors includes dopamine receptors, which bind to neuroleptic drugs used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc.

G-protein coupled receptors have been characterized as having seven putative transmembrane domains, designated as transmembrane domains 1-7 (“TM1,” “TM2,” “TM3,” “TM4,” “TM5,” “TM6,” and “TM7”). The domains are believed to represent transmembrane α-helices connected by extracellular or cytoplasmic loops. In each of the first two extracellular loops, most G-protein coupled receptors have single conserved cysteine residues forming disulfide bonds that are believed to stabilize functional protein structure. Phosphorylation (as well as lipidation, e.g., palmitylation or farnesylation) can influence signal transduction and potential phosphorylation sites lie within the third cytoplasmic loop and/or the carboxy-terminus. For several G-protein coupled receptors, such as the β-adrenoreceptor, phosphorylation by protein kinase A and/or specific receptor kinases mediates receptor desensitization.

Phosphorylation of cytoplasmic residues of G-protein coupled receptors has been identified as an important mechanism for the regulation of G-protein coupling. G-protein coupled receptors can be intracellularly coupled by heterotrimeric G-proteins to various intracellular enzymes, ion channels and transporters (see, Johnson et al., Endoc Rev, 1989, 10:317-331). Different G-protein α-subunits preferentially stimulate particular effectors to modulate various biological functions in a cell. This signaling pathway can be blocked, for example, by pertussis toxin (PTX) (Luster A D, 1998; Baggiolini, 1998).

As discussed above, chemokine-induced cell chemotaxis is mediated via a G_{αi}-linked signal transduction pathway. The chemokine, SDF-1α, provides one example of this signaling model. SDF-1α, causes immigration of subpopulations of leukocytes into sites of inflammation (Aiuti A et al. 1997; Bleul C C et al. 1996; Bleul C C et al., 1996; Oberlin E et al., 1996). Furthermore, mice engineered to be deficient in SDF-1α or its receptor, CXCR4, have abnormal development of hematopoietic tissues and B-cells due to the failure of fetal liver stem cells to migrate to bone marrow (Friedland J S, 1995; Tan J and Thestrup-Pedersen K, 1995; Corrigan C J and Kay A B, 1996; Qing M, et al, 1998; Ward S G et al. 1998). This movement is concentration-dependent, and is mediated via the CXCR4 receptor, Gαi protein and PI-3 kinase (Nature Medicine 2000; 6,543). The switch from a chemotactic to a fugetactic response in T cells is associated with intracytoplasmic levels of cyclic nucleotides and a differential sensitivity to tyrosine kinase inhibitors.

Methods for identification of the genes involved in modulation of cell movement through a gradient (e.g., genes involved in relevant G_{αi}-linked signal transduction pathways) have not been performed. Such methods would be useful for the identification of new therapeutic targets in diseases characterized by aberrant cellular movement.

### SUMMARY OF THE INVENTION

The invention is premised, in part, on the discovery that exposure of cells to a gradient results in changes in the gene expression profile of such cells. In addition, it has been unexpectedly found that movement of a cell through a gradient also induces changes in gene expression. In some cases, the gradients exist across the diameter of a cell such that the leading most edge of a cell is exposed to a different concentration of agent than is the lagging edge of the cell.

Thus, in one aspect, the invention provides a method for identifying a nucleic acid expressed in a concentration dependent manner, comprising determining a first nucleic acid expression profile of a first cell at a first position in an agent concentration gradient, determining a second nucleic acid expression profile of a second cell at a second position in the agent concentration gradient, and determining a difference between the first and second nucleic acid expression profiles. The first position in the agent concentration gradient corresponds to a first concentration of agent, and the second position in the agent concentration gradient corresponds to a second concentration of agent. Preferably, the second cell was genetically identical to the first cell prior to migration through the agent concentration gradient.

In some embodiments, at least the second cell has migrated through the agent concentration gradient. Therefore, the invention provides a method for identifying a nucleic acid expressed in a concentration dependent manner, comprising determining a first nucleic acid expression profile of a first cell at a first position in an agent concentration gradient, determining a second nucleic acid expression profile of a second cell that has migrated through the agent concentration gradient, and determining a difference between the first and second nucleic acid expression profiles.

In other embodiments, the neither cell has migrated through the agent concentration gradient, but at least the second cell is present in a gradient such that the agent concentration at one end of the cell is different from the agent concentration at the opposite end of the cell.

In one embodiment, the nucleic acid expression profile is a mRNA expression profile. In another embodiment, the mRNA expression profile is determined using PCR, RDA, Northern analysis, subtractive hybridization, or microarray analysis.

In one embodiment, the agent concentration gradient is a ligand concentration gradient. In another embodiment, the agent concentration gradient is a chemokine concentration gradient.

In yet another embodiment, the chemokine concentration gradient is selected from the group consisting of SDF-1α, SDF-1β, IP-10, MIG, GROα, GROβ, GROγ, IL-8, PF4, MCP, MIP-1α, MIP-1β, MIP-1γ (mouse), MCP-2, MCP-3, MCP-4, MCP-5 (mouse), RANTES, fractalkine, lymphotactin, CXC, IL-8, GCP-2, ENA-78, NAP-2, IP-10, MIG, I-TAC, SDF-1α, BCA-1, PF4, Bolekine, HCC-1, Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3), I-309, DC-CK1 (PARC, AMAC-1), TECK, CTAK, MPIF1 (MIP-3), MIP-5 (HCC-2), HCC-4 (NCC-4), C-10 (mouse), C Lymphotactin, and CX₃C Fracktelkine (Neurotactin) and ITAC concentration gradients.

The agent concentration gradient may be a cytokine concentration gradient. The cytokine concentration gradient may be selected from the group consisting of PAF, N-formylated peptides, C5a, LTB₄ and LXA₄, chemokines: CXC, IL-8, GCP-2, GRO, GROα, GROβ, GROγ, ENA-78, NAP-2, IP-10, MIG, I-TAC, SDF-1α, BCA-1, PF4, Bolekine, MIP-1α, MIP-1β, RANTES, HCC-1, MCP-1, MCP-2, MCP-3, MCP-4, MCP-5 (mouse), Leukotactin-1 (HCC-2, MIP-5), Eotaxin, Eotaxin-2 (MPIF2), Eotaxin-3 (TSC), MDC, TARC, SLC (Exodus-2, 6CKine), MIP-3a (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), MIP-1γ (mouse), C-10 (mouse), C Lymphotactin, and CX₃C Fracktelkine (Neurotactin) concentration gradients. The cytokine can be a member of the Cys-X-Cys family of chemokines (e.g., chemokines that bind to the CXCR-4 receptor). Preferred cytokines of the invention include SDF-1α, SDF-1β, met-SDF-113, IL-1, IL-2, IL-3, IL-4, IL-5, IL-6, IL-7, IL-10, IL-12, IL-15, IL-18, TNF, IFN-α, IFN-β, IFN-γ, granulocyte-macrophage colony stimulating factor (GM-CSF), granulocyte colony stimulating factor (G-CSF), macrophage colony stimulating factor (M-CSF), TGF-β, FLT-3 ligand, VEGF, DMDA, endothelin, and CD40 ligand.

In one embodiment, the first concentration of agent is a zero concentration of agent, and the second concentration of agent is a non-zero concentration of agent. In another embodiment, the first concentration of agent is greater than the second concentration of agent.

In one embodiment, the first cell has migrated through the agent concentration gradient. The migration through the agent concentration gradient may be fugetactic migration or chemotactic migration.

In one embodiment, the gradient is a step gradient. In another embodiment, the gradient is a continuous gradient. In yet another embodiment, the method further comprises a combination gradient, wherein at least one additional gradient co-exists with the first gradient.

In one embodiment, the first and second cells are adult cells. In preferred embodiments, the first and second cells are human cells. In one embodiment, the first and second cells are primary cells. In another preferred embodiment, first and second cells are hemopoietic cells, such as but not limited to T lymphocytes.

In another aspect, the invention provides a method for identifying a compound that can modulate cell migration in one or more agent concentration gradients comprising contacting a migratory cell in an agent concentration gradient with a test compound, determining the nucleic acid expression profile in the cell and identifying a change in expression of a gene expression product. Cell movement can be chemotaxis or fugetaxis and therefore, the gene expression product can be a chemotaxis or fugetaxis specific gene product.

In another aspect, the invention provides a method for inhibiting cell fugetaxis comprising contacting a cell undergoing or likely to undergo fugetaxis with an agent that inhibits a fugetaxis specific gene expression product in an amount effective to inhibit fugetaxis.

In one embodiment, the fugetaxis specific gene expression product is a nucleic acid or a peptide. In another embodiment, fugetaxis specific gene expression product is a signaling molecule. The signaling molecule may be selected from the group consisting of cell division cycle 42, annexin A3, Rap1 guanine nucleotide exchange factor, adenylate cyclase 1, JAK binding protein, and Rho GDP dissociation inhibitor alpha, but it is not so limited. In another embodiment, the signaling molecule is cell division cycle 42 (cdc42), ribosomal protein S6 kinase, BAI1-associated protein 2, GTPase regulator associated with FAK, protein kinase C-beta 1, phosphoinositide-specific phospholipase C-beta 1, nitric oxide synthase 1, phosphatidylinositol-4-phosphate 5-kinase, and MAP kinase kinase kinase kinase 4.

In another embodiment, the fugetaxis specific gene expression product is an extracellular matrix related molecule. In a related embodiment, the extracellular matrix related molecule may be selected from the group consisting of chitinase 3-like 1 (cartilage glycoprotein-39), carcinoembryonic antigen-related cell adhesion molecule 6, matrix metalloproteinase 8 (neutrophil collagenase), integrin cytoplasmic domain-associated protein 1, ficolin (collagenfibrinogen domain-containing) 1, and lysosomal-associated membrane protein 1, epithelial V-like antigen 1, vascular endothelial growth factor (VEGF), fibulin 1, carcinoembryonic antigen-related cell adhesion molecule 3, but it is not so limited.

In yet another embodiment, the fugetaxis specific gene expression product is a cytoskeleton related molecule. The cytoskeleton related molecule may be selected from the group consisting of ankyrin 1 (erythrocytic), S100 calcium-binding protein A12 (calgranulin C), plectin 1 (intermediate filament binding protein, 500 kD), and ankyrin 2 (neuronal), microtubule-associated protein RPEB3, microtubule-associated protein 1A like protein (MILP), capping protein (actin filament, gelsoline-like), but it is not so limited.

In still another embodiment, the fugetaxis specific gene expression product is a cell cycle molecule. The cell cycle molecule may be selected from the group consisting of v-kit Hardy-Zuckerman 4 feline sarcoma viral oncogene homolog, lipocalin 2 (oncogene 24p3), lectin, (galactoside-binding, galectin 3), RAB31 (member RAS oncogene family), disabled (Drosophila) homolog 2 (mitogen-responsive phosphoprotein), RAB9 (member RAS oncogene family, pseudogene 1), and growth differentiation factor 8, but it is not so limited.

In a further embodiment, the fugetaxis specific gene expression product is an immune response related molecule. The immune response related molecule may be selected from the group consisting of major histocompatibility complex (class II, DR alpha), S100 calcium-binding protein A8 (calgranulin A), small inducible cytokine subfamily A (Cys-Cys), eukaryotic translation initiation factor 5A, small inducible cytokine subfamily B (Cys-X-Cys) (member 6, granulocyte chemotactic protein 2), Fc fragment of IgG binding protein, CD24 antigen (small cell lung carcinoma cluster 4 antigen), cytochrome P450 (subfamily IVF, polypeptide 3, leukotriene B4 omega hydroxylase), MHC class II transactivator, T cell receptor (alpha chain), T cell activation (increased late expression), MKP-1 like protein tyrosine phosphatase, T cell receptor gamma constant 2, T cell receptor gamma locus, but it is not so limited.

In a further embodiment, the fugetaxis specific gene expression product is chemokine (C-X3-C) receptor 1.

In another aspect, the invention provides a method for inhibiting cell chemotaxis comprising contacting a cell undergoing or likely to undergo chemotaxis with an agent that inhibits a chemotaxis specific gene expression product in an amount effective to inhibit chemotaxis.

In one embodiment, the chemotaxis specific gene expression product is a nucleic acid or a peptide. In another embodiment, the cell is an immune cell.

In one embodiment, the contacting occurs in vivo in a subject having or at risk of having an abnormal immune response. In one embodiment, the abnormal immune response is an inflammatory response. In another embodiment, the abnormal immune response is an autoimmune response. The autoimmune response may be selected from the group consisting of rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus, but it is not so limited. In still another embodiment, the abnormal immune response is a graft versus host response.

In one embodiment, the chemotaxis specific gene expression product is a signaling molecule. In a related embodiment, the signaling molecule is selected from the group consisting of G protein-coupled receptor kinase 6, vaccinia related kinase 1, PTK2 protein tyrosine kinase 2, STAM-like protein containing SH3 and ITAM domains 2, signal-induced proliferation-associated gene 1, CD47 antigen (Rh-related antigen, integrin-associated signal transducer), and protein tyrosine phosphatase (non-receptor type 12). In another related embodiment, the signaling molecule is selected from the group consisting of PTK2 (focal adhesion kinase), MAP kinase kinase kinase kinase 2, guanine nucleotide binding protein, PT phosphatase (receptor), cdc42-binding protein kinase beta, Ral GEF (RalGPS1A), MAP kinase 7, autotaxin, inositol 1,4,5-triphosphate receptor, phosphoinositide-3-kinase gamma, PT phosphatase (non-receptor), RAS p21 protein activator (GAP), RAS guanyl releasing protein 2, and Arp23 complex 20 kDa subunit.

In one embodiment, the chemotaxis specific gene expression product is a extracellular matrix related molecule. In a related embodiment, the extracellular matrix related molecule is selected from the group consisting of spondin 1 (f-spondin, extracellular matrix protein), collagen type XVIII (alpha 1), CD31 adhesion molecule, tetraspan 3, glycoprotein A33, and angio-associated migratory cell protein.

In one embodiment, the chemotaxis specific gene expression product is a cytoskeleton related molecule. In a related embodiment, the cytoskeleton related molecule is selected from the group consisting of actin related protein 23 complex (subunit 4, 20 kD), tropomyosin 2 (beta), SWISNF related matrix associated actin dependent regulator of chromatin (subfamily a, member 5), spectrin beta (non-erythrocytic 1), myosin (light polypeptide 5, regulatory), keratin 1, plakophilin 4, and capping protein (actin filament, muscle Z-line, alpha 2).

In one embodiment, the chemotaxis specific gene expression product is a cell cycle molecule. In a related embodiment, the cell cycle molecule is selected from the group consisting of FGF receptor activating protein 1, v-maf musculoaponeurotic fibrosarcoma (avian) oncogene homolog, cyclin-dependent kinase (CDC2-like) 10, TGFB inducible early growth response 2, retinoic acid receptor alpha, anaphase promoting complex subunit 10, RAS p21 protein activator (GTPase activating protein, 3-Ins-1,3,4,5,-P4 binding protein), cell division cycle 27, programmed cell death 2, c-myc binding protein, mitogen-activated protein kinase kinase kinase 1, TGF beta receptor III (betaglycan, 300 kDa), and G1 to S phase transition 1.

In one embodiment, the chemotaxis specific gene expression product is an immune response related molecule. In a related embodiment, the immune response related molecule is selected from the group consisting of major histocompatibility complex class II DQ beta 1, bone marrow stromal cell antigen 2, Burkitt lymphoma receptor 1 (GTP binding protein, CXCR5), CD7 antigen (p41), Stat2 type a, T cell immune regulator 1, and interleukin 21 receptor.

In another aspect, the invention provides a method for promoting cell fugetaxis comprising contacting a cell with a non-chemokine agent that promotes fugetaxis in an amount effective to promote fugetaxis. In one embodiment, the contacting occurs in vivo in a subject having a disorder characterized by lack of fugetaxis. In one embodiment, the cell is a hematopoietic cell, such as a T lymphocyte. In another embodiment, the cell is a neural cell.

In another aspect, the invention provides a method for promoting cell chemotaxis comprising contacting a cell with a non-chemokine agent that promotes chemotaxis in an amount effective to promote chemotaxis. In one embodiment, the contacting occurs in vivo in a subject having a disorder characterized by lack of chemotaxis. In another embodiment, the cell is a hematopoietic cell, such as a T lymphocyte. In another embodiment, the cell is a neural cell.

The invention is also premised in part on various other findings. These include the finding that neutrophils migrate bi-directionally in response to IL-8. That is, neutrophils respond to low concentrations of IL-8 (e.g., 10 ng/ml to 500 ng/ml) by undergoing chemotaxis. Neutrophils respond to high concentration of IL-8 (e.g., 1 microgram/ml to 10 microgram/ml) by undergoing fugetaxis. Accordingly, the invention provides methods for modulating neutrophil migration by modulating the concentration of IL-8.

In one embodiment, the invention provides a method for promoting chemotaxis in a neutrophil comprising contacting a cell with IL-8 in an amount effective to promote chemotaxis by the neutrophil. In one embodiment, the contacting occurs in vivo in a subject having a disorder characterized by lack of neutrophil chemotaxis. Disorders characterized by lack of neutrophil chemotaxis include, but are not limited to, bacterial infections and granulomatous diseases (e.g., tuberculosis).

In one embodiment, the invention provides a method for promoting fugetaxis in a neutrophil comprising contacting a cell with IL-8 in an amount effective to promote fugetaxis by the neutrophil. In one embodiment, the contacting occurs in vivo in a subject having a disorder characterized by lack of neutrophil fugetaxis.

Disorders characterized by lack of neutrophil fugetaxis include, but are not limited to, inflammatory or immune mediated diseases, rejection of a transplanted organ or tissue, rheumatoid arthritis, automimmune diseases and asthma.

The invention further provides methods for identifying gene products that are modulated (i.e., either up regulated or down regulated) in response to IL-8 induced fugetaxis or chemotaxis. Thus, in a further aspect, the invention also provides methods for modulating the effects of IL-8 on neutrophils by inhibiting or enhancing the effects of IL-8 induced fugetaxis specific gene products or IL-8 induced chemotaxis specific gene products.

In another embodiment, the invention provides a method for inhibiting neutrophil chemotaxis comprising contacting a neutrophil undergoing or likely to undergo chemotaxis with IL-8 in an amount effective to inhibit or enhance expression of a chemotaxis specific gene expression product. In one embodiment, the contacting occurs in vivo in a subject having or at risk of having an abnormal immune response.

In a further embodiment, the chemotaxis specific gene expression product is an immune response related molecule. The immune response related molecule may be selected from the group consisting of IL-8, GCP-2, Gro-α, Gro β, Gro γ, CINC-1, CINC-2, ENA-78, NAP-2, LIX, SDF-1, IL-1α and IL-1β, C3a, C5a and leukotrienes.

The invention is further premised in part on the finding that IL-8 induced chemotaxis of neutrophils is selectively inhibited by the PIK3 inhibitor wortmannin, causing cells to undergo fugetaxis to all concentrations of IL-8. Accordingly, in one embodiment, the invention provides methods for inhibiting IL-8 induced chemotaxis of neutrophils (conversely enhancing IL-8 induced fugetaxis of neutrophils) by administering to a subject in need thereof an effective amount of wortmannin. The effective amount of wortmannin is that amount effective to selectively inhibit IL-8 induced chemotaxis of neutrophils and optionally to enhance neutrophil fugetaxis in the presence of IL-8. The method can also be performed with other species of this genus.

In another embodiment, the invention provides a method for inhibiting neutrophil fugetaxis comprising contacting a neutrophil undergoing or likely to undergo fugetaxis with IL-8 in an amount effective to inhibit or enhance expression of a fugetaxis specific gene expression product. In one embodiment, the contacting occurs in vivo in a subject having or at risk of having an abnormal immune response.

In a further embodiment, the fugetaxis specific gene expression product is an immune response related molecule. The immune response related molecule may be selected from the group consisting of IL-8, GCP-2, Gro-α, Gro β, Gro γ, CINC-1, CINC-2, ENA-78, NAP-2, LIX, SDF-1, IL-1α and IL-1β, C3a, C5a and leukotrienes.

The invention is further premised in part on the finding that IL-8 induced fugetaxis of neutrophils is selectively inhibited by alternative PI3K inhibitor LY294002, causing cells to chemotax to all concentrations of IL-8. Accordingly, in one embodiment, the invention provides methods for inhibiting IL-8 induced fugetaxis of neutrophils (and conversely enhancing IL-8 induced chemotaxis of neutrophils) by administering to a subject in need thereof an effective amount of PI3K inhibitor LY294002. The effective amount of LY294002 is that amount effective to selectively inhibit IL-8 induced fugetaxis of neutrophils and optionally to enhance neutrophil chemotaxis in the presence of IL-8. The method can also be performed with other species of this genus.

These and other objects of the invention will be described in further detail in connection with the detailed description of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings, in which:

FIG. 1 is a schematic showing chemotaxis, chemokinesis, and fugetaxis in a T cell migration assay.

FIGS. 2A and 2B are schematics showing putative downstream events that result following chemokine engagement at the cell surface.

FIGS. 3 through 8 indicate the genes that are significantly (p value≦to 0.05; fold change≧1.7) differentially regulated under different gradient conditions of SDF-1. Gen Bank Accession Numbers are provided to further describe the identified gene products.

FIG. 3 depicts Table 1, indicating genes that are differentially regulated in Medium vs. Chemokinesis gradients of SDF-1. Positive values are upregulated in Chemokinesis; Negative values are down regulated in Chemokinesis; p≦0.05.

FIG. 4 depicts Table 2, indicating genes that are differentially regulated in Fugetaxis vs. Chemotaxis gradients of SDF-1. Positive values are upregulated in Fugetaxis; Negative values are up regulated in Chemotaxis; p≦0.05.

FIG. 5 depicts Table 3, indicating genes that are differentially regulated in Chemokinesis vs. Chemotaxis gradients of SDF-1. Positive values are upregulated in Chemotaxis; Negative values are downregulated in Chemotaxis; p≦0.05.

FIG. 6 depicts Table 4, indicating genes that are differentially regulated in Chemokinesis vs. Fugetaxis gradients of SDF-1. Positive values are upregulated in Fugetaxis; Negative values are downregulated in Fugetaxis; p≦0.05.

FIG. 7 depicts Table 5, indicating genes that are differentially regulated in Medium vs. Chemotaxis gradients of SDF-1. Positive values are upregulated in Chemotaxis; Negative values are downregulated in Chemotaxis; p≦0.05.

FIG. 8 depicts Table 6, indicating genes that are differentially regulated in Medium vs. Fugetaxis gradients of SDF-1. Positive values are upregulated in Chemotaxis; Negative values are downregulated in Chemotaxis; p≦0.05.

FIG. 9 depicts Table 7, indicating actin/cytoskeletal, extracellular matrix/adhesion, T-cell activation and migration related proteins differentially regulated under different gradient conditions of SDF-1.

FIGS. 10A through 10P depict the migration of human neutrophils in a continuous (0, 12 mm, 120 nM or 1.2 mM) linear gradient of IL-8 in microfabricated devices. Cell migration in uniform concentrations or continuous gradients of IL-8 (tracked with the assistance of MetaMorph software) is depicted in FIGS. 10E through 10H. Normalized cell concentration across the migration channel (measured by MetaMorph software) is depicted in FIGS. 10I through L. Distribution of movement vector angles for all cells for all time points is depicted in FIGS. 10M through P.

FIGS. 11A and 11B depict lots of mean speeds (11A) and mean square displacement (11B) for cells tracked over time in videos of cells migrating in the absence of IL-8 or defined as continuous linear gradients of the chemokine at peak concentrations of 12 nM, 120 nM and 1.2 mM.

FIG. 12 depicts effect of SB225002 on directional migration of neutrophils towards and away from IL-8.

FIG. 13 depict effects of chemokine signal transduction pathway inhibitors on directional human neutrophil migration in defined continuous gradients of IL-8.

FIGS. 14A through 14I depict intravital microscopic quantitation of rat neutrophil migration in response to continuous diffusive gradients of the IL-8 orthologue, CINC-1. Diffusive continuous gradients are mathematically modeled and depicted in FIGS. 14A, 14B and 14C. A single photomicrograph derived from the first frame of the timelapse video is depicted in 14D (Video 5), 14E (Video 6) and 14F (Video 7). FIGS. 14G, 14H and 14I depict cell tracks normalized to an origin and again use the same color code as in FIG. 14 for directional and random cell movement.

FIG. 15 depicts quantitative parameters defined for measuring the directional bias and orientation of cellular movement of cells tracked in videos of neutrophils migrating in the absence of IL-8 (No-IL-8), a constant concentration of chemokine (120 nM IL-8 no gradient), and three continuous linear gradient conditions with peak concentrations of IL-8, 12 nM, 120 nM and 1.2 mM within microfabricated devices (Table 8).

FIG. 16 depicts quantitated motility parameters for rat neutrophils migrating in response to diffusive continuous CINC-1 gradients in vivo (Table 9).

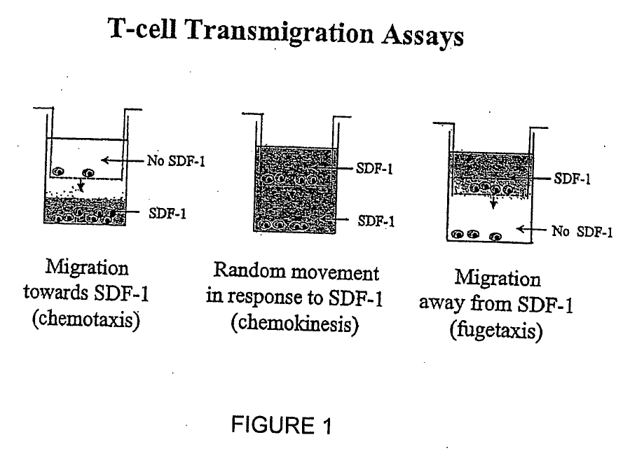

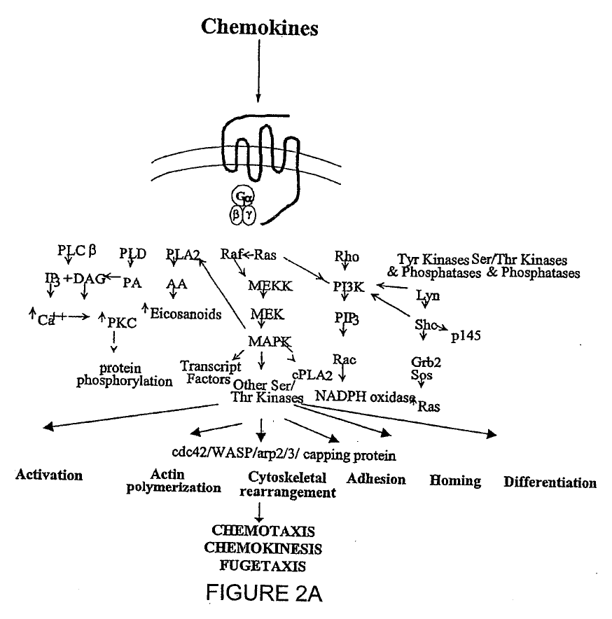

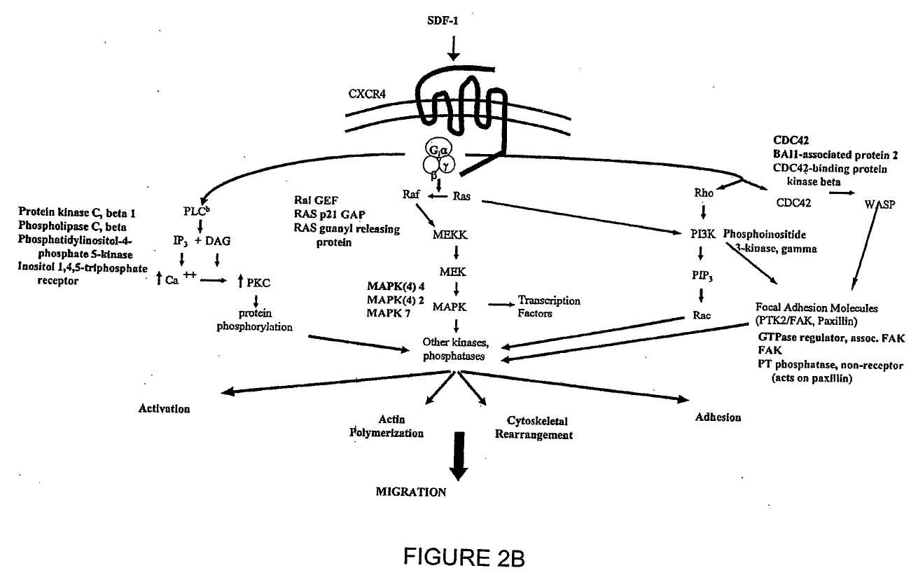

### DETAILED DESCRIPTION OF THE INVENTION

The invention is premised in part on the discovery that cells exposed to a gradient undergo gene expression changes associated with the presence of the gradient and movement through the gradient. It has been unexpectedly found that exposure of cells to an agent gradient causes differential gene expression in cells so exposed as compared to cells exposed to a uniform agent concentration (i.e., no gradient). As a result gene expression profiles during or following exposure to gradients is significantly different from those observed during or following exposure to uniform agent concentrations. Furthermore, gene expression profiles are dependent on the structure of the gradient. That is, if the gradient is oriented such that the cell is attracted to an agent source (an attractant gradient or a chemoattractant agent), the gene expression profile will be different than if the gradient is oriented such that the cell is repelled from the agent source (a fugetactic gradient or agent). Gene expression profiles for cells exposed to a fugetactic gradient are clearly distinct from those seen in chemotactic gradients. As an example, when a cell is exposed to an SDF-1 (CXCL12) gradient, it begins to differentially express genes involved in chemokine signal transduction depending upon whether it is migrating towards or away from an agent source.

Definitions

As used in accordance with terms appearing herein, the following definitions are provided:

An “agent” is a diffusible substance that can alter gene expression in a migratory cell, either alone or in combination with other agents. Preferably, the agent is an attractant or repellant of a migratory cell.

An “agent concentration gradient” is a gradually increasing concentration of an agent, wherein the location of highest agent concentration is at the agent source.

A “continuous gradient” is a physiologically relevant, continuous agent concentration range over a fixed distance.

A “step gradient” comprises agent concentrations that descend or ascend abruptly to another concentration of the agent.

An “agent source” is the point at which the concentration of an agent is highest. As a cell migrates towards the source, it is moving towards higher agent concentration, and as it migrates away from the source, it is moving towards lower agent concentration.

A “ligand” is a molecule, such as a protein, lipid or cation, capable of binding to another molecule for which it has affinity, such as a receptor. A ligand is therefore one member of a binding interaction or association.

“Chemotactic migration” or “chemotaxis” is the movement of a migratory cell toward an agent source (i.e., towards a higher concentration of agent).

“Fugetactic migration” or “fugetaxis” is the movement of a migratory cell away from an agent source (i.e., towards a lower concentration of agent).

“Chemokinetic migration” or “chemokinesis” is the random movement of cells irrespective of a gradient.

A “cytokine” is generic term for all extracellular proteins or peptides that mediate cell-cell communication, often with the effect of altering the activation state of cells.

A “chemokine” is a cytokine with a conserved cysteine motif and which can serve as an attractant

A “signaling molecule” is a molecule involved in the transduction of a signal cascade from one compartment of the cell to another (e.g., in the case of cell movement, a signaling molecule can be involved in the transduction of a signal cascade from the cell membrane to the actin cytoskeleton).

A “cytoskeleton related molecule” is a component of the cytoskeleton, which is a system of protein filaments (e.g., actin filaments, integrins, microtubules and intermediate filaments) in the cytoplasm of a eukaryotic cell that gives the shape and capacity for cellular movement.

A “cell cycle molecule” is a molecule involved in regulating, initiating or halting the reproductive cycle of a cell, which is the cycle by which a cell duplicates its contents and divides into two.

An “extracellular matrix related molecule” is a molecule that is a component of the extracellular matrix, which is a network of structural elements, such as polysacchrides and proteins, secreted by cells.

An “immune response related molecule” is a molecule involved in the generation, propagation or termination of an immune response, which is a response by an immune cell to an antigen.

An “immune cell” is a cell of hematopoietic origin that is involved in the specific recognition of antigens. Immune cells include, but are not limited to T-cells, B-cells, NK cells, dendritic cells. monocytes and macrophages.

“Primary cells” are cells directly obtained from living normal or diseased tissues.

An “inflammatory cell” is a cell contributing to an immune response including, but not limited to, neutrophils, basophils, eosinophils and mast cells.

Additional definitions and descriptions appear in context below.

Other aspects of the invention are disclosed in, or are obvious from the following disclosure and are within the ambit of the invention.

Methods of the Invention

The methods of the invention can be used to determine the differences between cells that undergo chemotaxis versus those that undergo fugetaxis, or differences between cells that undergo either chemotaxis or fugetaxis versus those that undergo chemokinesis (i.e., random movement). In some instances, gene expression profiles of cells undergoing chemokinesis are considered “background” and thus subtracted from both chemotactic and fugetactic gene expression profiles.

These expression differences identify further mediators of chemotaxis and fugetaxis and provide novel targets that can be affected in order to modulate directed cell movement. In some instances, these newly identified targets can be administered to cells directly. Alternatively, the newly identified targets can be up-regulated or down-regulated in ways that are independent of actual exposure to a chemotactic or fugetactic gradient. These include introduction of nucleic acids into cells (e.g., antisense or gene therapy), and exposure of cells to compounds that modulate the newly identified targets (e.g., agonists or antagonists).

Yet another unexpected finding of the invention is the observation that cells are capable of sensing not only differences in agent concentration, but also differences in agent concentration along their length. Previous work relating to concentration gradients and cells compared cells in differing concentrations. The invention is based in part on the finding that cells respond to changes in concentration, but also are able to sense their position in a gradient based on the difference in agent concentration along the length of the cell. That is, a cell can sense its position in a gradient, and thereby modulate its expression profile, by sensing that its opposite ends are exposed to different agent concentrations.

In one aspect, the invention provides a method for identifying a nucleic acid expressed in an agent concentration dependent manner. The method comprises determining a first nucleic acid expression profile of a first cell at a first position in an agent concentration gradient, determining a second nucleic acid expression profile of a second cell at a second position in the agent concentration gradient, and determining a difference between the first and second nucleic acid expression profiles, wherein the first position in the agent concentration gradient corresponds to a first concentration of agent, and the second position in the agent concentration gradient corresponds to a second concentration of agent.

In some embodiments, at least the second cell has migrated through the agent concentration gradient. Therefore, the invention provides a method for identifying a nucleic acid expressed in a concentration dependent manner, comprising determining a first nucleic acid expression profile of a first cell at a first position in an agent concentration gradient, determining a second nucleic acid expression profile of a second cell that has migrated through the agent concentration gradient, and determining a difference between the first and second nucleic acid expression profiles.

In another embodiment, the second cell is positioned in the gradient such that a gradient exists along the length (or diameter) of the cell. In other words, the agent concentration at one end of the cell (e.g., the leading edge of the cell) is different that the agent concentration at the opposite end of the cell (e.g., the lagging edge of the cell). Thus, the method may be performed by placing a cell into a preformed concentration gradient, or allowing the cell to move through the concentration gradient, depending upon the application and information desired.

The chemotactic, fugetactic or chemokinetic response can be measured as described herein, or according to the transmigration assays described in greater detail in U.S. Pat. No. 6,448,054 B1, and in U.S. Pat. No. 5,514,555, entitled: “Assays and therapeutic methods based on lymphocyte chemoattractants,” issued May 7, 1996, to Springer, T A, et al.). Other suitable methods will be known to one of ordinary skill in the art and can be employed using only routine experimentation.

Agent concentration gradients can be established using an agent source. The agent source is the location in a gradient having the highest concentration of agent, and is generally the location at which agent is supplied to establish the gradient. Agent can be continually supplied or the source can be over-saturated with agent that there is no need for replenishment of the agent during the course of the screening. In preferred embodiments, the gradient is established and it remains constant throughout the screening process. That is, the concentration differential between the agent source and the end of the gradient is constant, as is the concentration differential between different locations in the gradient.

In some embodiments, the first concentration of agent is a zero concentration of agent, and the second concentration of agent is a non-zero concentration of agent, while in other embodiments the first concentration of agent is greater than the second concentration of agent. The cells might migrate through the gradient, and in these embodiments, one or both cells will migrate through the agent concentration gradient. The migration may be fugetactic migration, or chemotactic migration. The gradient can be either a step gradient or a continuous gradient, although a continuous gradient is preferred in some embodiments. In still another embodiment, there may be a second gradient overlapped onto the first gradient. In an important embodiment, the first cell has undergone chemotaxis and the second cell has undergone fugetaxis, and the expression profiles of these cells are compared.

The nucleic acid expression profile can be an RNA (preferably an mRNA) profile or it can be a protein profile. Depending upon which expression product is being analysed, the method of analysis and quantitation of the expression product will differ. If the nucleic acid expression product is itself a nucleic acid, such as an RNA (e.g., mRNA), then it can be quantitated using a number of methods including but not limited to Northern analysis, reverse-transcriptase polymerase chain reaction (RT-PCR), subtractive hybridization, differential display, representational difference analysis and cDNA microarray analysis. In some embodiments, the nucleic acids are harvested from the cells and analyzed without the need for in vitro amplification.

The differentially expressed molecule can be identified in a number of ways. If the expression product is a nucleic acid (i.e., an mRNA), then it may be identified using techniques such as subtractive hybridization (including suppression subtractive hybridization), differential display, representational difference analysis, or microarray analysis (e.g., Affymetrix chip analysis). These techniques have been reported in the literature, and thus one of ordinary skill will be familiar with these. (See, for example, Methods Enzymol 303:349-380, 1999; Ying and Lin in Biotechniques 26:966-8, 1999; Zhao et al., J Biotechnol 73:35-41, 1999; and Blumberg and Belmonte in Methods Mol Biol 97:555-574, 1999.) Sequences isolated in this screening process can then be sequenced and compared to the GenBank non-redundant and EST databases using the BLAST algorithm.

Another important technique for identifying differentially expressed transcripts involves DNA chip technology and cDNA microarray hybridization. This technique is able to analyze hundreds if not thousands of coding sequences at a time. Standard and custom-made DNA chips are now commercially available from manufacturers such as Affymetrix and InCyte. These approaches have evolved to the extent that high throughput screening for difference sequences can be readily accomplished. (Von Stein, et al., Nucleic Acids Res 25:2598-602, 1997; Carulli, et al., J Cell Biochem Suppl 30-31:286-96, 1998) One of the major advantages of DNA chip technology is that no RNA amplification is required.

If the nucleic acid expression product is a protein, then it may be identified using, for example, gel electrophoresis separation followed by Coomassie Blue staining. In this latter approach, differences between the experimental cell and a control may be revealed by the presence or absence of stained protein bands. Further separation, sequencing and cloning of these “difference bands” would then be required, all of which are within the realm of the ordinary artisan. Other approaches can similarly be used to identify and/or quantitate nucleic acid expression products that are proteins, and these include but are not limited to immunohistochemistry, Western analysis, and fluorescence activated cytometry.

The agent to be used in establishing a gradient is not intended to be limiting. Any agent that induces a change in gene expression profile would be suitable. The agent can be a ligand, resulting in a ligand concentration gradient. Accordingly, the ligand can also be a receptor. In some preferred embodiments, the agent is a molecule that induces chemotaxis or fugetaxis.

The agent may be a cytokine (including a chemokine). For a further description of a cytokine, see Human Cytokines: Handbook for Basic & Clinical Research (Aggrawal, et al. eds., Blackwell Scientific, Boston, Mass. 1991) (which is hereby incorporated by reference in its entirety for all purposes). Examples of cytokines include PAF, N-formylated peptides, C5a, LTB₄ and LXA₄, chemokines: CXC, IL-8, GCP-2, GRO, GROα, GROβ, GROγ, ENA-78, NAP-2, IP-10, MIG, I-TAC, SDF-1α, BCA-1, PF4, Bolekine, MIP-1α, MIP-1β, RANTES, HCC-1, MCP-1, MCP-2, MCP-3, MCP-4, MCP-5 (mouse), Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), MIP-1γ (mouse), C-10 (mouse), C Lymphotactin, and CX₃C Fracktelkine (Neurotactin). The cytokine can be a member of the Cys-X-Cys family of chemokines (e.g., chemokines that bind to the CXCR-4 receptor). Preferred cytokines of the invention include SDF-1α, SDF-1β, met-SDF-1β, IL-1, IL-2, IL-3, IL-4, IL-5, IL-6, IL-7, IL-10, IL-12, IL-15, IL-18, TNF, IFN-α, IFN-β, IFN-γ, granulocyte-macrophage colony stimulating factor (GM-CSF), granulocyte colony stimulating factor (G-CSF), macrophage colony stimulating factor (M-CSF), TGF-β, FLT-3 ligand, VEGF, DMDA, endothelin, and CD40 ligand. This list is not meant to be exhaustive and one of ordinary skill will be able to identify other cytokines that can be used in the methods of the invention. In certain embodiments, the cytokine is a cytokine with chemoattractant and/or chemokinetic properties.

The agent may be a chemokine. Chemokines, or chemoattractant cytokines, are a family of small proteins with a conserved cysteine motifs. These small proteins have been implicated in a wide range of disease states, such as acute and chronic inflammatory processes, angiogenesis, leukocyte migration, regulation of cell proliferation and maturation, hematopoiesis, viral replication, and other immunoregulatory functions. Chemokines are expressed by a number of different cells and have distinct but overlapping cellular targets.

Chemokines have been classified into four subgroups, depending on the nature of the spacing of two highly-conserved cysteine amino acids that are located near the amino terminus of the polypeptide. The first chemokine subgroup is referred to as “CXC”; the second subgroup is referred to as “CC”; the third chemokine subgroup is referred to as “CX3C”; and the fourth chemokine subgroup is referred to as “C”. Within these subgroups, the chemokines are further divided into related families that are based upon amino acid sequence homology. The CXC chemokine families include the IP-10 and MIG family; the GROα, GROβ, and GROβ family; the interleukin-8 (IL-8) family; and the PF4 family. The CC chemokine families include the monocyte chemoattractant protein (MCP) family; the family including macrophage inhibitory protein-1α (MIP-1α), macrophage inhibitory protein-1β (MIP-1β), and regulated on activation normal T cell expressed (RANTES). The stromal cell-derived factor 1α (SDF-1α) and stromal cell-derived factor 1β (SDF-1β) represent a chemokine family that is approximately equally related by amino acid sequence homology to the CXC and CC chemokine subgroups. The CX3C chemokine family includes fractalkine; the C chemokine family includes lymphotactin.

In general, the CXC chemokines are bound by members of the CXCR class of receptors; the CC chemokines are bound by the CCR class of receptors; the CX3C chemokines are bound by the CX3CR class of receptors; and the C chemokines are bound by the CR class of receptors. The majority of chemokine receptors are transmembrane spanning molecules which belong to the family of G-protein-coupled receptors. Many of these receptors couple to guanine nucleotide binding proteins to transmit cellular signals.

Chemokines and receptor expression is upregulated during inflammatory responses and cellular activation. Chemokines, through binding to their respective receptors, have been shown to be involved in a number of physiologic conditions. For instance, chemokines of the CXC group, like interleukin-8, can stimulate angiogenesis, while platelet factor-4, growth-related oncogene-β (GRO-β) and interferon-γ induced protein-10 (IP-10) inhibit endothelial cell proliferation and angiogenesis. Interleuken-8 stimulates endothelial cell proliferation and chemotaxis in vitro, and appears to be a primary inducer of macrophage induced angiogenesis. It was shown that the activities of these chemokines are dependent on the NH₂-terminal amino acid sequence (Streiter et al., J. Biol. Chem., 270:27348-27357). SDF-1, another CXC chemokine, is active in the recruitment and mobilization of hematopoietic cells from the bone marrow, as well as the attraction of monocytes and lymphocytes.

The agent can be any molecule, either naturally occurring or synthetically produced. The agent may be isolated from a biological sample such as a biological fluid. Biological fluids include but are not limited to synovial fluid, cerebral spinal fluid, fallopian tube fluid, seminal fluid, ocular fluid, pericardial fluid, pleural fluid, inflammatory exudate, and ascitic fluid. The agent may also be present in a tumor cell culture supernatant, tumor cell eluate and/or tumor cell lysate.

In preferred embodiments, the agent is a molecule that induces chemotaxis or fugetaxis. In another embodiment, the agent is a fugetactic agent at one concentration and a chemotactic agent at a lower concentration.

The cells to be used in the methods of the invention are not limited to cell type, provided it has migratory capacity. An example of a cell with migratory capacity is a hematopoietic cell, such as neutrophils, basophils, eosinophils, monocytes, macrophages, dendritic cells, T cells, and the like. In some embodiments, the cell with migratory capacity is a neural cell. In further embodiments, the cell with migratory capacity is an epithelial cell. In yet further embodiments, the cell with migratory capacity is a mesenchymal cell. In some embodiments, the cell with migratory capacity is an embryonic stem cell. In certain embodiments, the cell with migratory capacity is a germ cell. In important embodiments, the cells are mammalian cells, such as human cells. In important embodiments, the cells are primary human T cells. In other embodiments, the cells are neural cells such as neurons capable of undergoing chemotaxis or fugetaxis for example in response to a neurotransmitter.

Cells which express chemokine receptors include migratory cells such as lymphocytes, granulocytes, and antigen-presenting cells (APCs) that are believed to participate in immune responses or that may release other factors to mediate other cellular processes in vivo. The presence of a chemokine gradient serves to attract migratory cells which express the chemokine receptors. For example, migratory cells can be attracted by a chemokine gradient to a particular site of inflammation, at which location they play a role in further modifying the immune response.

“Immune cells” as used herein are cells of hematopoietic origin that are involved in the specific recognition of antigens. Immune cells include antigen presenting cells (APCs), such as dendritic cells or macrophages, B cells, T cells, etc. “Mature T cells” as used herein include T cells of a CD4^{lo}CD8^{hi}CD69⁺TCR⁺, CD4^{hi}CD8^{lo}CD69⁺TCR⁺, CD4⁺CD45⁺RA⁺, CD4⁺CD3⁺RO⁺, and/or CD8⁺CD3⁺ RO⁺ phenotype. Fugetaxis may play a role in the emigration of T cells from the thymus during development.

Cells of “hematopoietic origin” include, but are not limited to, pluripotent stem cells, multipotent progenitor cells and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage. The hematopoietic cells may be derived from a tissue such as bone marrow, peripheral blood (including mobilized peripheral blood), umbilical cord blood, placental blood, fetal liver, embryonic cells (including embryonic stem cells), aortal-gonadal-mesonephros derived cells, and lymphoid soft tissue. Lymphoid soft tissue includes the thymus, spleen, liver, lymph node, skin, tonsil and Peyer's patches. In other embodiments, the “hematopoietic origin” cells may be derived from in vitro cultures of any of the foregoing cells, and in particular in vitro cultures of progenitor cells.

Cells of neural origin, include neurons and glia, and/or cells of both central and peripheral nervous tissue that express RR/B (see, U.S. Pat. No. 5,863,744, entitled: “Neural cell protein marker RR/B and DNA encoding same,” issued Jan. 26, 1999, to Avraham, et al.). Work in Xenopus indicates that neurons and growth cones respond to netrins. Neurons are expected to respond either by chemotaxing or fugetaxing to the presence of neurotransmitters. Cells of epithelial origin, include cells of a tissue that covers and lines the free surfaces of the body. Such epithelial tissue includes cells of the skin and sensory organs, as well as the specialized cells lining the blood vessels, gastrointestinal tract, air passages, ducts of the kidneys and endocrine organs. Cells of mesenchymal origin include cells that express typical fibroblast markers such as collagen, vimentin and fibronectin. Cells involved in angiogenesis are cells that are involved in blood vessel formation and include cells of epithelial origin and cells of mesenchymal origin. An embryonic stem cell is a cell that can give rise to cells of all lineages; it also has the capacity to self-renew. A germ cell is a cell specialised to produce haploid gametes. It is a cell further differentiated than a stem cell that can still give rise to more differentiated germ-line cells. The cell may be a eukaryotic cell or a prokaryotic cell.

In some embodiments, the cells used in the screening assays are adult cells. Preferably, they are human cells. They may be primary cells (e.g., directly harvested cells), or they may be secondary cells (including cells from a cell line).

The invention in one aspect identifies differential expression products that are upregulated or downregulated during chemokinesis (i.e., random movement), as compared to cells in medium alone. The identification of these products can be exploited in instances where it is desired to inhibit or facilitate cell movement. Products upregulated during chemokinesis include the signaling molecules PTK2 (focal adhesion kinase) (upregulated by a value of 6.88) and regulator of G-protein signaling 10 (upregulated by a value of 2.53). Products downregulated during chemokinesis include the signaling molecules phospholipase C beta 3 (downregulated by a value of 2.54), RAS p21 protein activator (GAP) 3 (downregulated by a value of 2.20), RAS guanyl releasing protein 2 (calcium/DAG) (downregulated by a value of 2.16), G protein-coupled receptor kinase 6 (downregulated by a value of 2.15), Rho-specific GEF (p114) (downregulated by a value of 1.70) and protein kinase C substrate 80K-H (downregulated by a value of 1.70). Knowledge of these products at a minimum allows for the identification of products that are specifically differentially regulated in response to either chemotaxis or fugetaxis (i.e., it is possible to distinguish between those products that are impacted by purposeful directional movement rather than simply random movement). The data provided in the tables below are generally presented as levels of expression of a particular gene product relative to the level of that gene product when the cell from which it is derived is placed in medium alone or is allowed to undergo chemokinesis. Knowledge of these products also leads to methods for inhibiting or stimulating movement of cells, depending upon the desired effect. It is possible that many of these products are required in chemotaxis and fugetaxis and thus provide another target for preventing or stimulating these directional migrations. In this way, these “chemokinesis” specific products can be thought of as the “housekeeping products” of cell movement in general (i.e., they are required for movement, regardless of whether the movement is directional or not). Agents that stimulate these products include agonists and nucleic acids that encode the products, but are not so limited. Agents that inhibit these products include antagonists, antibodies, and antisense nucleic acids, but are not so limited.

In another aspect, the invention provides a method for identifying a compound that can modulate cell migration in one or more agent concentration gradients comprising contacting a migratory cell in an agent concentration gradient with a test compound, determining the nucleic acid expression profile in the cell and identifying a change in expression of a gene expression product. Cell movement can be chemotaxis or fugetaxis and therefore, the gene expression product can be a chemotaxis or fugetaxis specific gene product. A test compound is any compound that is thought to potentially modulate chemotaxis or fugetaxis.

The invention further provides methods of modulating chemotaxis and fugetaxis. As used herein, modulate means to affect or change, and includes stimulation or inhibition. In order to modulate chemotaxis or fugetaxis, cells are contacted or exposed to agents that are differential expression products as identified according to the invention, or that impact upon the differential expression products. The differential expression products identified according to the invention are thus additional, previously unrecognized targets that can be manipulated in order to modulate chemotaxis or fugetaxis.

The ability to modulate chemotaxis and fugetaxis is important for manipulating bodily processes, such as but not limited to immune responses, thymic emigration, and neural outgrowth (for example, in response to neurotransmitters). In some instances, it will be desirable to inhibit an immune response that is occurring or is likely to occur in a subject. Examples include subjects that have asthma, allergy, autoimmune diseases such as rheumatoid arthritis, infections that are detrimental due to the immune response that is formed in response (e.g., RSV infection, particularly in infants), inflammatory conditions, graft versus host disease (GVHD), and the like. In other instances, it will be desirable to promote or stimulate an immune response where a subject is likely to benefit from such a response. These subjects include those that have or are likely to develop infections (e.g., bacterial infections, viral infections, fungal infection, parasitic infections), and those that have or are likely to develop a cancer in order to heighten immune surveillance for cancer cells. Other subjects include those that are diagnosed as having an impaired immune response, particularly where the defect lies in the inability of immune cells to respond to chemotactic factors.

Accordingly, in one embodiment, a cell undergoing or likely to undergo fugetaxis is contacted or exposed to an agent that inhibits a fugetaxis specific gene expression product in an amount effective to inhibit fugetaxis. The fugetaxis inhibiting agent can act at the nucleic acid or protein level. Fugetaxis specific gene expression products are those that are upregulated in response to fugetaxis as compared to their level when the cells are moving randomly (i.e., chemokinesis) or when the cells are chemotaxing. Since these products are upregulated in response to fugetaxis, fugetaxis may be inhibited by blocking the activity of these products using a number of methods known in the art, including but not limited to antisense and antibody approaches. The products can also be targeted in order to modulate chemotaxis, as one of ordinary skill will understand.

The signaling molecules can be but are not limited to cell division cycle 42, annexin A3, Rap1 guanine nucleotide exchange factor, adenylate cyclase 1, JAK binding protein, and Rho GDP dissociation inhibitor alpha. In another embodiment, the signaling molecule is cell division cycle 42 (cdc42), ribosomal protein S6 kinase, BAI1-associated protein 2, GTPase regulator associated with FAK, protein kinase C-beta 1, phosphoinositide-specific phospholipase C-beta 1, nitric oxide synthase 1, phosphatidylinositol-4-phosphate 5-kinase, and MAP kinase kinase kinase kinase 4.

The extracellular matrix related molecules can be but are not limited to Chitinase 3-like 1 (cartilage glycoprotein-39), carcinoembryonic antigen-related cell adhesion molecule 6, matrix metalloproteinase 8 (neutrophil collagenase), integrin cytoplasmic domain-associated protein 1, ficolin (collagenfibrinogen domain-containing) 1, epithelial V-like antigen 1, vascular endothelial growth factor (VEGF), fibulin 1, carcinoembryonic antigen-related cell adhesion molecule 3, and lysosomal-associated membrane protein 1.

The cytoskeleton related molecules can be but are not limited to Ankyrin 1 (erythrocytic), S100 calcium-binding protein A12 (calgranulin C), plectin 1 (intermediate filament binding protein, 500 kD), microtubule-associated protein RPEB3, microtubule-associated protein 1A like protein (MILP), capping protein (actin filament, gelsolin-like), and ankyrin 2 (neuronal).

The cell cycle molecules can be but are not limited to V-kit Hardy-Zuckerman 4 feline sarcoma viral oncogene homolog, lipocalin 2 (oncogene 24p3), lectin, (galactoside-binding, galectin 3), RAB31 (member RAS oncogene family), disabled (Drosophila) homolog 2 (mitogen-responsive phosphoprotein), RAB9 (member RAS oncogene family, pseudogene 1), and growth differentiation factor 8.

The immune response related molecules can be but are not limited to major histocompatibility complex (class II, DR alpha), S100 calcium-binding protein A8 (calgranulin A), small inducible cytokine subfamily A (Cys-Cys), eukaryotic translation initiation factor 5A, small inducible cytokine subfamily B (Cys-X-Cys) (member 6, granulocyte chemotactic protein 2), Fc fragment of IgG binding protein, CD24 antigen (small cell lung carcinoma cluster 4 antigen), cytochrome P450 (subfamily IVF, polypeptide 3, leukotriene B4 omega hydroxylase), MHC class II transactivator, T cell receptor (alpha chain), T cell activation (increased late expression), MKP-1 like protein tyrosine phosphatase, T cell receptor gamma constant 2, T cell receptor gamma locus.

The fugetaxis specific gene expression product may also be chemokine (C—X3-C) receptor 1.

The invention further provides a method for inhibiting cell chemotaxis. The method involves contacting a cell undergoing or likely to undergo chemotaxis with an agent that inhibits a chemotaxis specific gene expression product in an amount effective to inhibit chemotaxis.

The chemotaxis inhibiting agent can act at the nucleic acid or protein level. Chemotaxis specific gene expression products are those that are upregulated in response to chemotaxis as compared to their level in chemokinesis or in fugetaxis. Since these products are upregulated in response to chemotaxis, chemotaxis may be inhibited by blocking the activity of these products using a number of methods known in the art, including but not limited to antisense and antibody approaches.

The signaling molecules can be but are not limited to G protein-coupled receptor kinase 6, vaccinia related kinase 1, PTK2 protein tyrosine kinase 2, STAM-like protein containing SH3 and ITAM domains 2, signal-induced proliferation-associated gene 1, CD47 antigen (Rh-related antigen, integrin-associated signal transducer), and protein tyrosine phosphatase (non-receptor type 12). The signaling molecule may also be selected from the group consisting of PTK2 (focal adhesion kinase), MAP kinase kinase kinase kinase 2, guanine nucleotide binding protein, PT phosphatase (receptor), cdc42-binding protein kinase beta, Ral GEF (RalGPS1A), MAP kinase 7, autotaxin, inositol 1,4,5-triphosphate receptor, phosphoinositide-3-kinase gamma, PT phosphatase (non-receptor), RAS p21 protein activator (GAP), RAS guanyl releasing protein 2, and Arp23 complex 20 kDa subunit.

The extracellular matrix related molecules can be but are not limited to spondin 1 (f-spondin, extracellular matrix protein), collagen type XVIII (alpha 1), CD31 adhesion molecule, tetraspan 3, glycoprotein A33, and angio-associated migratory cell protein.

The cytoskeleton related molecules can be but are not limited to actin related protein 23 complex (subunit 4, 20 kD), tropomyosin 2 (beta), SWISNF related matrix associated actin dependent regulator of chromatin (subfamily a, member 5), spetrin beta (non-erythrocytic 1), myosin (light polypeptide 5, regulatory), keratin 1, plakophilin 4, and capping protein (actin filament, muscle Z-line, alpha 2).

The cell cycle molecules can be but are not limited to FGF receptor activating protein 1, v-maf musculoaponeurotic fibrosarcoma (avian) oncogene homolog, cyclin-dependent kinase (CDC2-like) 10, TGFB inducible early growth response 2, retinoic acid receptor alpha, anaphase promoting complex subunit 10, RAS p21 protein activator (GTPase activating protein, 3-Ins-1,3,4,5, -P4 binding protein), cell division cycle 27, programmed cell death 2, c-myc binding protein, mitogen-activated protein kinase kinase kinase 1, TGF beta receptor III (betaglycan, 300 kDa), and G1 to S phase transition 1.

The immune response related molecules can be but are not limited to major histocompatibility complex class II DQ beta 1, bone marrow stromal cell antigen 2, Burkitt lymphoma receptor 1 (GTP binding protein, CXCR5), CD7 antigen (p41), Stat2 type a, T cell immune regulator 1, and interleukin 21 receptor.

The contacting of cells with the inhibitory or stimulatory agents of the invention can occur in vivo. And as mentioned above the subject receiving the agent will vary depending upon the type of agent being administered. Thus, in one embodiment where the method is intended to inhibit chemotaxis, the subject is one having or at risk of having an abnormal immune response.

The abnormal immune response may be an inflammatory response or an autoimmune response but it is not so limited. Autoimmune disease is a class of diseases in which an subject's own antibodies react with host tissue or in which immune effector T cells are autoreactive to endogenous self peptides and cause destruction of tissue. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, uveitis, rheumatic fever, Guillain-Barre syndrome, psoriasis, and autoimmune hepatitis.

According to still another aspect of the invention, a method is provided for promoting fugetaxis. The method involves contacting a cell with a non-chemokine agent that promotes fugetaxis in an amount effective to promote fugetaxis. In one embodiment, the contacting occurs in vivo in a subject having a disorder characterized by abnormal fugetaxis. As used herein, a non-chemokine agent is an agent that is not a chemokine such as those recited above. The non-chemokine agent is preferably one of the downstream targets of fugetaxis identified according to the invention, or it is an agonist thereof.

The invention further provides a method for promoting chemotaxis. The method involves contacting a cell with a non-chemokine agent that promotes chemotaxis in an amount effective to promote chemotaxis. In one embodiment, the contacting occurs in vivo in a subject having a disorder characterized by lack of chemotaxis. The non-chemokine agent is preferably one of the downstream targets of fugetaxis identified according to the invention, or it is an agonist thereof.

As stated above, in some instances, modulating occurs by administration of nucleic acids (e.g., in antisense therapy), or proteins or peptides (e.g., antibody therapy). In some embodiments, the nucleic acids or proteins/peptides are isolated. In still further embodiments, the nucleic acids or proteins/peptides are substantially pure.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein with respect to proteins/peptides, the term “isolated” means separated from its native environment in sufficiently pure form so that it can be manipulated or used for any one of the purposes of the invention. Thus, isolated means sufficiently pure to be used (i) to raise and/or isolate antibodies, (ii) as a reagent in an assay, or (iii) for sequencing, etc.

The term “substantially pure” means that the nucleic acid or protein/peptide is essentially free of other substances with which it may be found in nature or in vitro systems, to an extent practical and appropriate for their intended use. Substantially pure polypeptides may be produced by techniques well known in the art. As an example, because an isolated protein may be admixed with a pharmaceutically acceptable carrier in a pharmaceutical preparation, the protein may comprise only a small percentage by weight of the preparation. The protein is nonetheless isolated in that it has been separated from many of the substances with which it may be associated in living systems, i.e. isolated from certain other proteins.

According to another aspect, the invention provides compositions and methods relating to attracting or repelling immune cells to or from a material surface. These aspects of the invention involve coating or loading material surfaces alternatively with the chemotactic inhibiting agents, the chemotactic stimulating agents, the fugetactic inhibiting agents, or the fugetactic stimulating agents provided herein. “Material surfaces” as used herein, include, but are not limited to, dental and orthopedic prosthetic implants, artificial valves, and organic implantable tissue such as a stent, allogeneic and/or xenogeneic tissue, organ and/or vasculature.

Implantable prosthetic devices have been used in the surgical repair or replacement of internal tissue for many years. Orthopedic implants include a wide variety of devices, each suited to fulfill particular medical needs. Examples of such devices are hip joint replacement devices, knee joint replacement devices, shoulder joint replacement devices, and pins, braces and plates used to set fractured bones. Some contemporary orthopedic and dental implants, use high performance metals such as cobalt-chrome and titanium alloy to achieve high strength. These materials are readily fabricated into the complex shapes typical of these devices using mature metal working techniques including casting and machining.

The material surface is coated with an amount of agent effective to repel or attract cells (e.g., immune cells), depending upon the desired therapeutic effect. In important embodiments, the material surface is part of an implant. In important embodiments, in addition to a fugetactic agent, the material surface may also be coated with a cell growth potentiating agent, an anti-infective agent, and/or an anti-inflammatory agent.

A cell-growth potentiating agent as used herein is an agent which stimulates growth of a cell and includes growth factors such as PDGF, EGF, FGF, TGF, NGF, CNTF, and GDNF.

An anti-infectious agent as used herein is an agent which reduces the activity of or kills a microorganism and includes: Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosformycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifiratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarnoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Omidazole; Pentisomicin; and Sarafloxacin Hydrochloride.

An anti-inflammatory agent is an agent that reduces or inhibits altogether an inflammatory response in vivo and includes Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Tainiflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

According to one aspect of the invention, a method of inhibiting migration of immune cells to a specific site in a subject is provided. The method involves locally administering to a specific site in a subject in need of such treatment an agent that promotes fugetaxis in an amount effective to inhibit migration of immune cells to the specific site in a subject.

In one important embodiment, the invention provides a method of inhibiting migration of immune cells to a site of inflammation in the subject. “Inflammation” as used herein, is a localized protective response elicited by a foreign (non-self) antigen, and/or by an injury or destruction of tissue(s), which serves to destroy, dilute or sequester the foreign antigen, the injurious agent, and/or the injured tissue. Inflammation occurs when tissues are injured by viruses, bacteria, trauma, chemicals, heat, cold, or any other harmful stimuli. In such instances, the classic weapons of the immune system (T cells, B cells, macrophages) interface with cells and soluble products that are mediators of inflammatory responses (neutrophils, eosinophils, basophils, kinin and coagulation systems, and complement cascade).

A typical inflammatory response is characterized by (i) migration of leukocytes at the site of antigen (injury) localization; (ii) specific and nonspecific recognition of “foreign” and other (necrotic/injured tissue) antigens mediated by B and T lymphocytes, macrophages and the alternative complement pathway; (iii) amplification of the inflammatory response with the recruitment of specific and nonspecific effector cells by complement components, lymphokines and monokines, kinins, arachidonic acid metabolites, and mast cell/basophil products; and (iv) macrophage, neutrophil and lymphocyte participation in antigen destruction with ultimate removal of antigen particles (injured tissue) by phagocytosis.

According to yet another aspect of the invention, a method of enhancing an immune response in a subject having a condition that involves a specific site, is provided. The method involves locally administering to a specific site in a subject in need of such treatment an agent that inhibits fugetaxis or stimulates chemotaxis in an amount effective to inhibit immune cell-specific fugetactic activity at a specific site in the subject. In some embodiments, the specific site is a site of a pathogenic infection. Efficient recruitment of immune cells to help eliminate the infection is therefore beneficial.

In certain embodiments, the specific site is a germ cell containing site. In this case the recruitment of immune cells to these specific sites will help eliminate unwanted germ cells, and/or implanted and nonimplanted embryos. In further embodiments, co-administration of contraceptive agents other than anti-fugetactic agents is also provided.

In further embodiments, the specific site is an area immediately surrounding a tumor. Since most of the known tumors escape immune recognition, it is beneficial to enhance the migration of immune cells to the tumor site. In further embodiments, co-administration of anti-cancer agents other than anti-fugetactic agents is also provided. Non-anti-fugetactic anti-cancer agents include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cluorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n 1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Podofilox; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogernanium Hydrochloride; Spiromustine; Spiroplatin; Streptonigrin; Streptozocin; Sulofenur; Talisomycin; Taxotere; Tecogalan Sodium; Tegaflur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; and Vinrosidine Sulfate.

In some embodiments, the fugetaxis stimulating, fugetaxis inhibiting, chemotaxis stimulating or chemotaxis inhibiting agents of the invention are administered substantially simultaneously with other therapeutic agents. By “substantially simultaneously,” it is meant that the agents are administered to the subject close enough in time, so that the other therapeutic agents may exert a potentiating effect on migration inhibiting or stimulating activity of the fugetactic or chemotactic agent. The fugetactic or chemotactic agent may be administered before, at the same time, and/or after the administration of the other therapeutic agent.

The methods provided herein in some instances may be carried out by administration of antisense molecules in order to block transcription or translation of nucleic acid expression products. As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the identification of molecules that are upregulated in fugetaxis or chemotaxis (see the Tables herein), one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11): 1116-1118, 1995. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules together with pharmaceutically acceptable carriers. Antisense oligonucleotides may be administered as part of a pharmaceutical composition. In this latter embodiment, it is preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient.

The compositions, as described above, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result. In some cases this is a local (site-specific) reduction of inflammation. In other cases, it is inhibition of tumor growth and/or metastasis. In still other embodiments, the effective amount is that amount sufficient for stimulating an immune response leading to the inhibition of an infection, or a cancer.

Generally, doses of active compounds of the present invention would be from about 0.01 mg/kg per day to 1000 mg/kg per day. It is expected that doses ranging from 50-500 mg/kg will be suitable. A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. When peptides are used therapeutically, in certain embodiments a desirable route of administration is by pulmonary aerosol. Techniques for preparing aerosol delivery systems containing peptides are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences 18th edition, 1990, pp 1694-1712; incorporated by reference). Those of skill in the art can readily determine the various parameters and conditions for producing antibody or peptide aerosols without resort to undue experimentation.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

The agents may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The invention in other aspects includes pharmaceutical compositions of the agents. When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Various techniques may be employed for introducing nucleic acids of the invention (e.g., antisense nucleic acids) into cells, depending on whether the nucleic acids are introduced in vitro or in vivo in a host. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Other delivery systems can include time-release, delayed release or sustained release delivery systems (collectively referred to herein as controlled release). Such systems can avoid repeated administrations of the fugetactic agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) difusional systems in which an active component permeates at a controlled rate from a polymer such as described in U.S. Pat. Nos. 3,832,253, and 3,854,480.

A preferred delivery system of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μM can encapsulate large macromolecules. RNA, DNA, and intact virions can be encapsulated within the aqueous interior and be delivered to cells in a biologically active form (Fraley, et al., Trends Biochem. Sci., (1981) 6:77). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one important embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the fugetactic agents described herein are encapsulated or dispersed within the biocompatible, preferably biodegradable polymeric matrix disclosed in PCT/US/03307.

The polymeric matrix preferably is in the form of a microparticle such as a microsphere (wherein an agent is dispersed throughout a solid polymeric matrix) or a microcapsule (wherein an agent is stored in the core of a polymeric shell). Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used. Preferably when an aerosol route is used the polymeric matrix and fugetactic agent are encompassed in a surfactant vehicle. The polymeric matrix composition can be selected to have both favorable degradation rates and also to be formed of a material which is bioadhesive, to further increase the effectiveness of transfer. The matrix composition also can be selected not to degrade, but rather, to release by diffusion over an extended period of time.

In another important embodiment the delivery system is a biocompatible microsphere that is suitable for local, site-specific delivery. Such microspheres are disclosed in Chickering et al., Biotech. And Bioeng., (1996) 52:96-101 and Mathiowitz et al., Nature, (1997) 386:410-414.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, fugetactic agents are delivered using a bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In addition, important embodiments of the invention include pump-based hardware delivery systems, some of which are adapted for implantation. Such implantable pumps include controlled-release microchips. A preferred controlled-release microchip is described in Santini, J T Jr., et al., Nature, 1999, 397:335-338, the contents of which are expressly incorporated herein by reference.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In certain embodiments, the agents of the invention are delivered directly to the site at which there is inflammation, e.g., the joints in the case of a subject with rheumatoid arthritis, the blood vessels of an atherosclerotic organ, etc. For example, this can be accomplished by attaching an agent (nucleic acid or polypeptide) to the surface of a balloon catheter; inserting the catheter into the subject until the balloon portion is located at the site of inflammation, e.g. an atherosclerotic vessel, and inflating the balloon to contact the balloon surface with the vessel wall at the site of the occlusion. In this manner, the compositions can be targeted locally to particular inflammatory sites to modulate immune cell migration to these sites. In another example the local administration involves an implantable pump to the site in need of such treatment. Preferred pumps are as described above. In a further example, when the treatment of an abscess is involved, the fugetactic agent may be delivered topically, e.g., in an ointment/dermal formulation. Optionally, the agents are delivered in combination with other therapeutic agents (e.g., anti-inflammatory agents, immunosuppressant agents, etc.).

The invention will be more fully understood by reference to the following examples. These examples, however, are merely intended to illustrate the embodiments of the invention and are not to be construed to limit the scope of the invention.

### EXAMPLE 1

Example 1 describes experiments and findings that demonstrate that bi-directional migratory response of T cells to specific gradients of the chemokine are associated with differential changes in the expression of genes encoding proteins involved in SDF-1/CXCR4 signal transduction pathway.

Methods

Primary murine or human T cells were exposed to specific gradients of SDF-1 to induce chemotaxis or fugetaxis in vitro and in vivo. The Zigmund/Hirsch chamber and microfabricated devices as well as a murine model of allergic peritonitis were used to establish defined SDF-1 gradients in vitro and in vivo, respectively. Purified T cells were generated from these systems and unamplified RNA examined using genomic array technology (Affymetrix). These results were validated by RT-PCR and Northern blotting. Control experiments were performed on T cells which had not been exposed to SDF-1 or which had been exposed to the chemokine in the absence of a gradient.

Cell Cultures: CD4+CD45+RA cells were obtained from peripheral blood Buffy coat samples from healthy donors.

Transwell Assays: Transwell assays were done using 0.4 μm pore size filters (23 mm diameter, with polycarbonate membrane; Corning Inc., New York). 10×106 cells suspended in 0.5% FBS-containing IMDM were added to the upper chamber of the transwell. To create positive, negative, uniform, and absent gradients, either of 0.5% FBS IMDM medium alone or medium plus SDF-1α.

Total RNA Extraction: Total RNA was extracted from all samples using Gibco's TRIzol protocol (GIBC-BRL, Life Technologies, Rockville, Md.) with 1 mL Trizol per 10-20×10⁶ cells. Total RNA was brought to a concentration of 1 μg/μL and 5-10 μg were used on the Affymetrix chips.

cRNA Preparation and Chip Hybridization Conditions: cRNA probes were prepared according to the GeneChip Expression Analysis Technical Manual and as described previously (Warrington et al. 2000). Briefly, 5-10 μg of total RNA was used to synthesize double-stranded cDNA using SuperScript Choice System (GIBCO-BRL) and a T7-(dT)-24 primer (Geneset Oligos, La Jolla, Calif.). The cDNA was purified by phenol/chloroform/isoamyl alcohol extraction with Phase Lock Gel (5Prime 3Prime, Boulder, Colo.) and concentrated by EtOH precipitation. In vitro transcription produced biotin-labeled cRNA using a BioArray HighYield RNA Transcript Labeling Kit (Affymetrix) according to the manufacturer's instructions. cRNA was linearly amplified with T7 polymerase, the biotinylated cRNA was cleaned with RNeasy Mini kit (Qiagen), and 20 μg of labeled cRNA was fragmented (Warrington et al. 2000). The fragmented cRNA was hybridized to the microarray for 16 hours at 45° C. with a constant rotation of 60 rpm in the GeneChip Hybridization Oven 640 (Affymetrix). After being washed, the arrays were stained with streptavidin-phycoerythrin (Molecular Probes, Eugene, Oreg.) and amplified by biotinylated anti-streptavidin (Vector Laboratories, Burlingame, Calif.) using the GeneChip Fluidics Station 400 (Affymetrix), and scanned on the GeneArray scanner (Affymetrix). The intensity for each feature of the array was captured with Affymetrix GeneChip Software v5.0, according to standard Affymetrix procedures (Warrington et al. 2000).

Statistical Analysis of Expression Data: To enable comparison between experiments, Affymetrix image (.cel) files were loaded into the Rosetta Resolver v4.0 Expression Data Analysis System and normalized according to the Resolver error model (see Waring et al. 2001, Lock et al. 2002 for description).

Q-PCR Verification of Gene Targets: Total RNA from primary T cells was isolated, purified, and quantified as described above. QRT-PCR was performed using the Brilliant One-Setp QRT-PCR kit (Stratagene, La Jolla, Calif.) containing SYBR Green I (1:30,000, Molecular Probes), forward and reverse primers (50 nM each; Invitrogen), and sample RNA (amount was variable, depending on the transcript abundance).

Results

Chips of the same condition were combined using Rosetta's Resolver error-model based software, as described in the Methods. The combined experiments were then compared between each other in different combinations in order to address distinct sub-components of the hypothesis: M/M—basal conditions; CM—chemokinesis; CT—chemotaxis in positive SDF-1 gradient; and FT—fugetaxis in negative SDF-1 gradient.

The gene expression profile for T cells which underwent chemotaxis differed from the profile generated for T cells which underwent fugetaxis in response to gradients of SDF-1 in several significant respects. Cluster analysis of gene expression demonstrated that genes encoding molecules known to be involved in SDF-1 signal transduction were significantly and differentially expressed (p≦0.05 for 1.7 to 21 fold changes in RNA expression) when cells which had undergone fugetaxis or chemotaxis were compared. Of particular note, these differentially expressed genes encoded members of the G-protein-coupled receptor kinase, cellular tyrosine kinase, PI-3 kinase and Rho GTPase cascades as well as the cyclic nucleotide metabolic pathway. The gene expression profile for control T cells exposed to SDF-1 in the absence of a gradient also differed from profiles generated from cells responding to gradients of the chemokine.

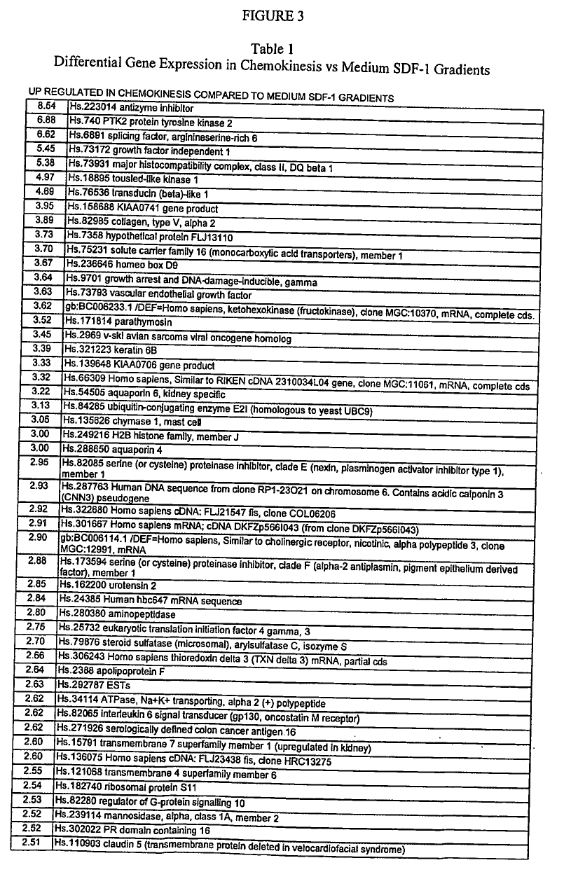

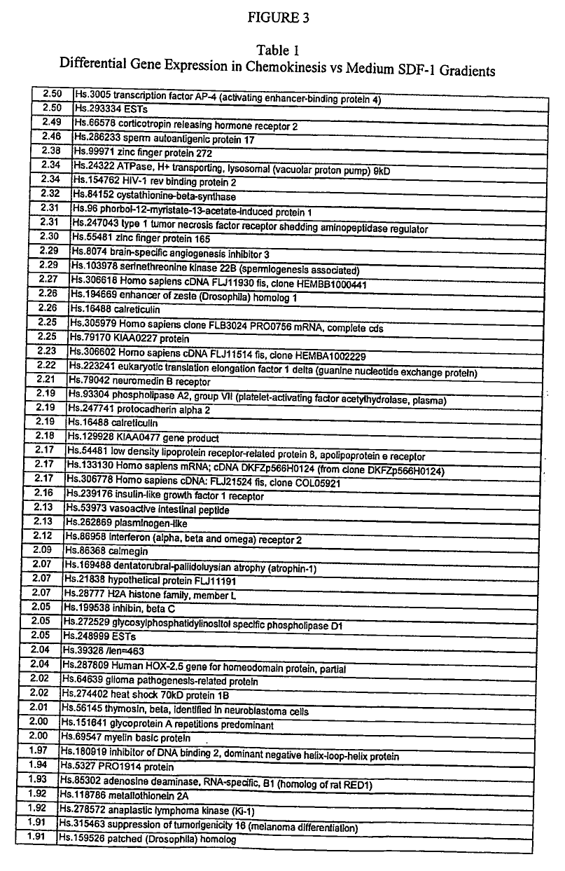

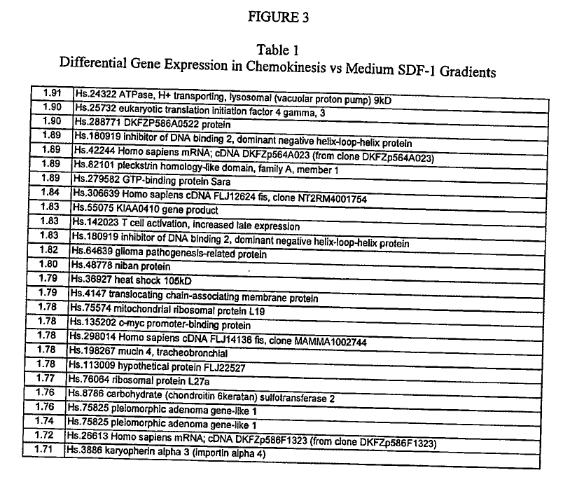

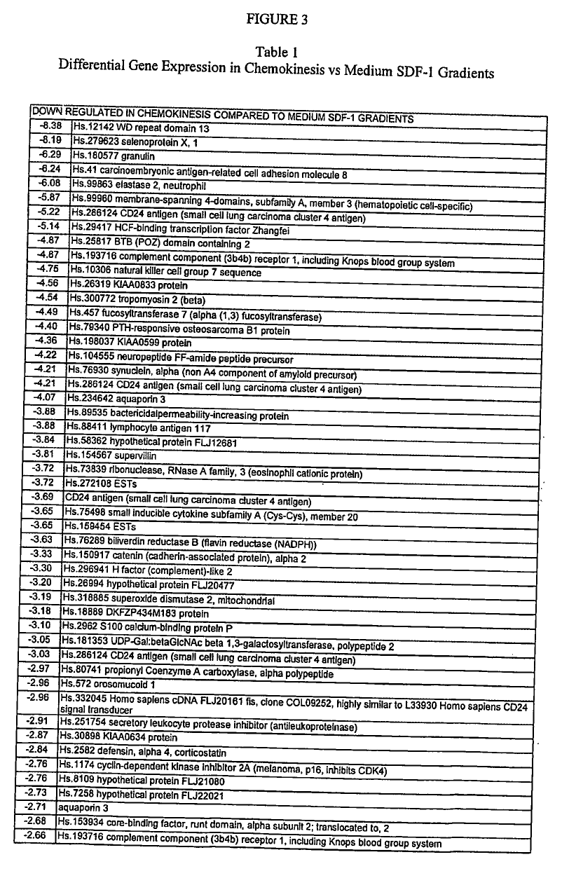

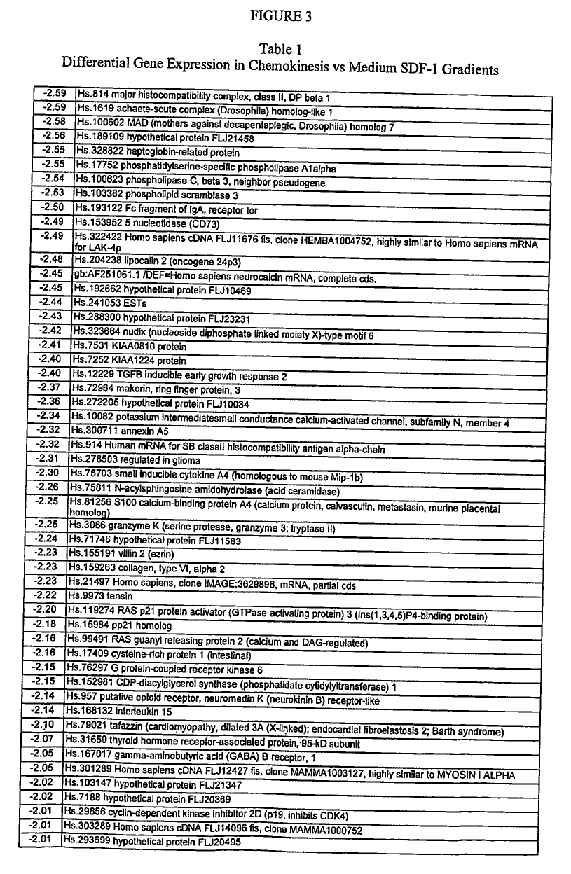

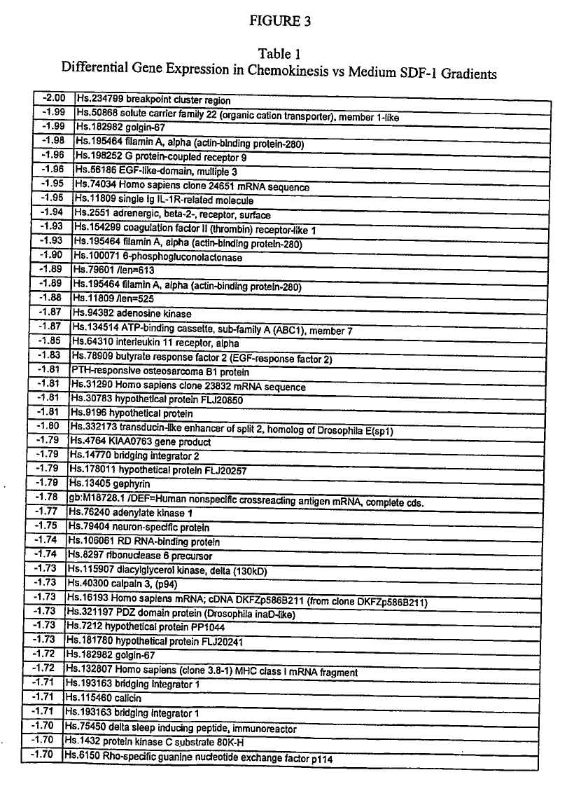

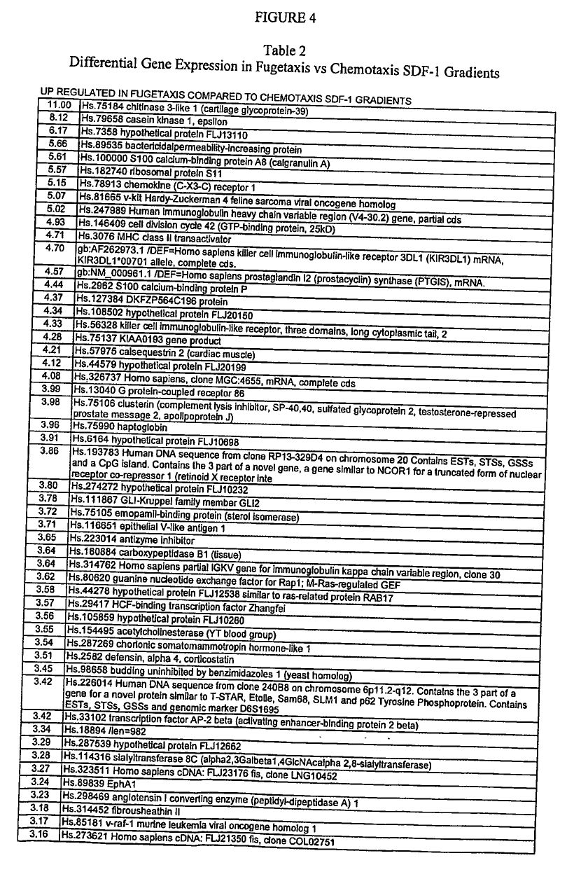

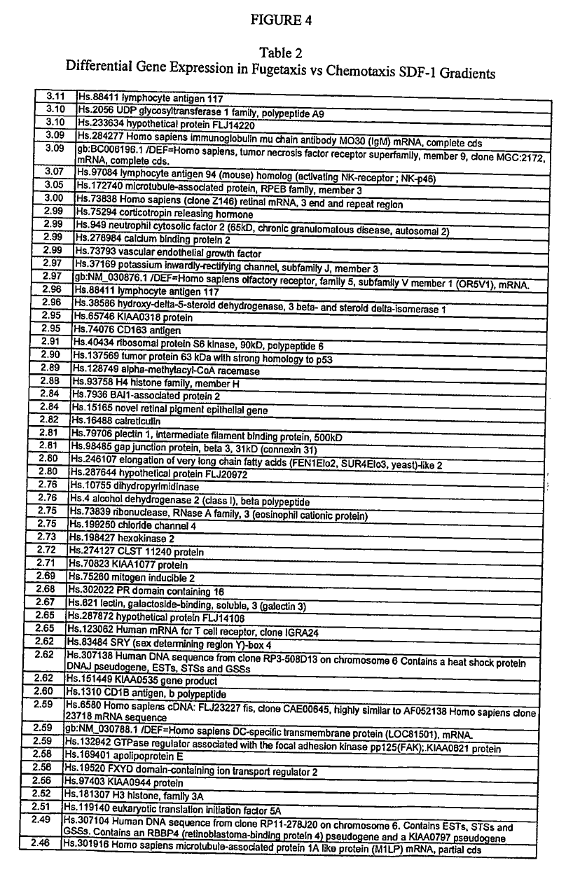

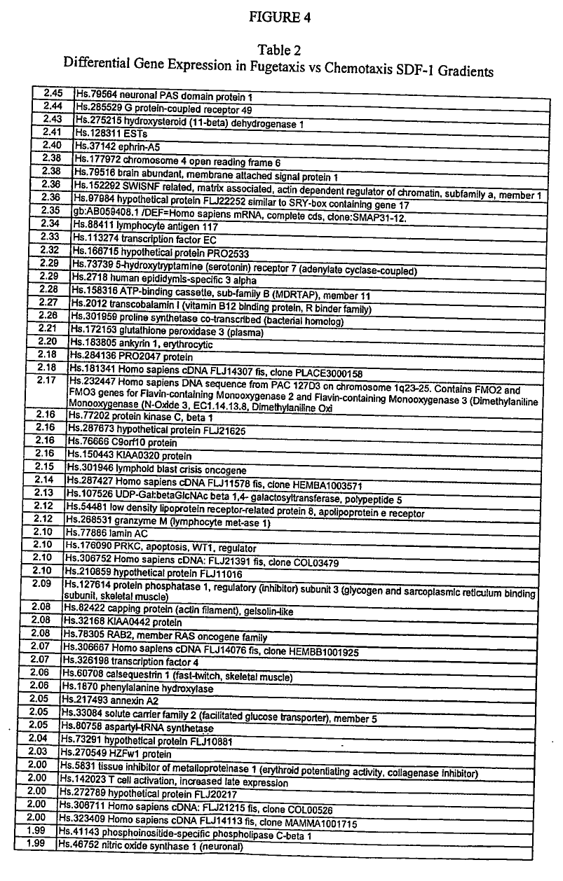

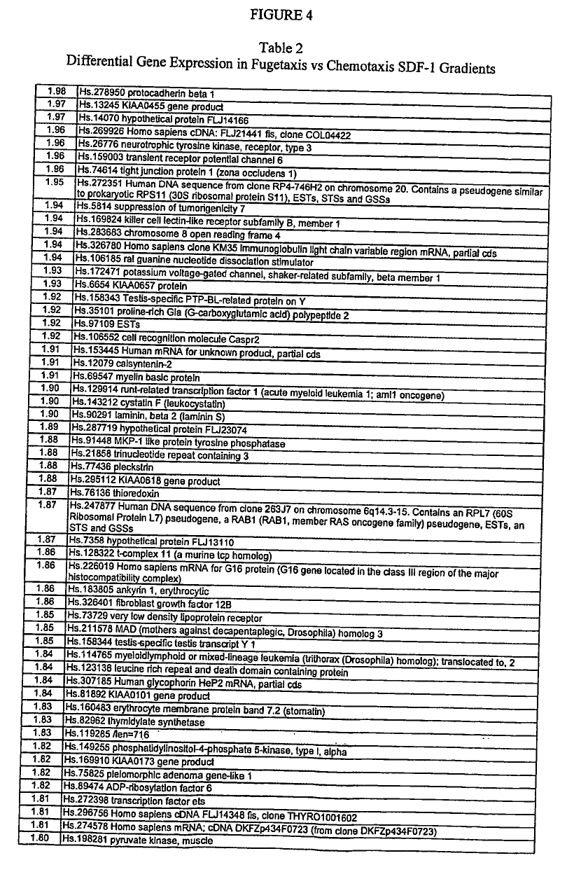

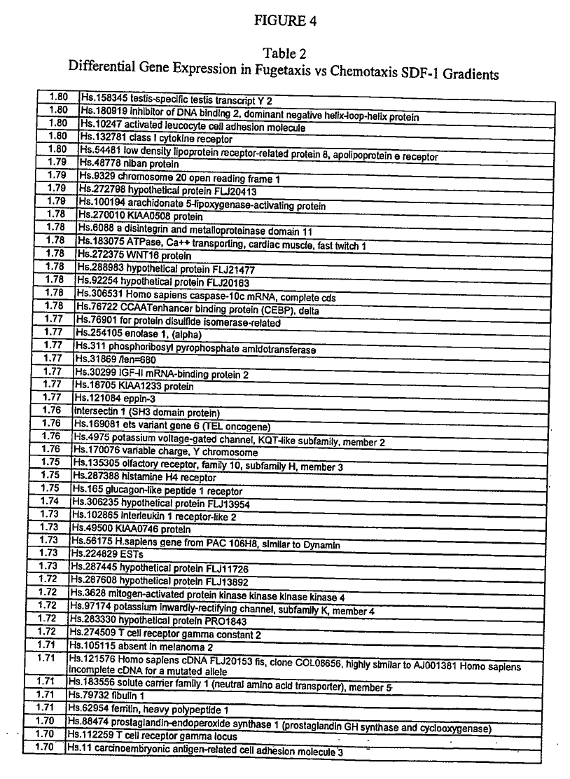

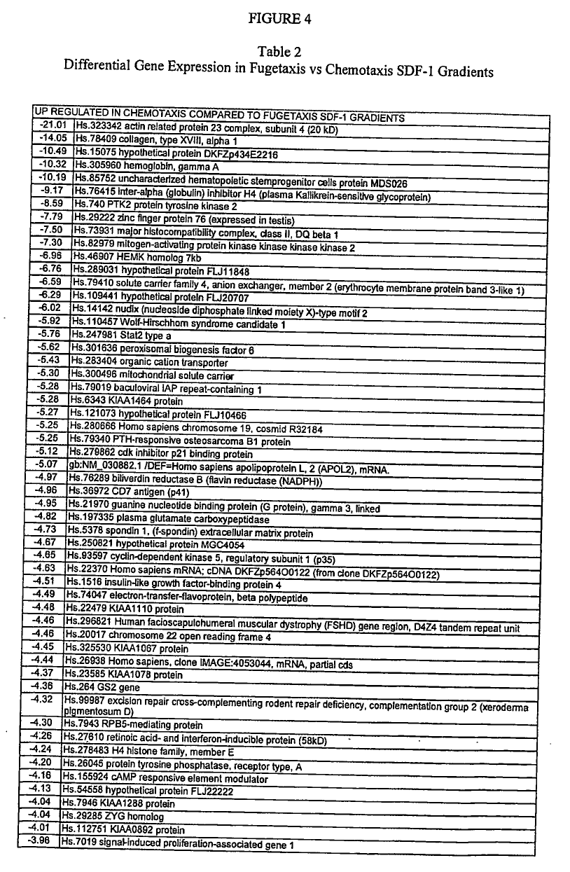

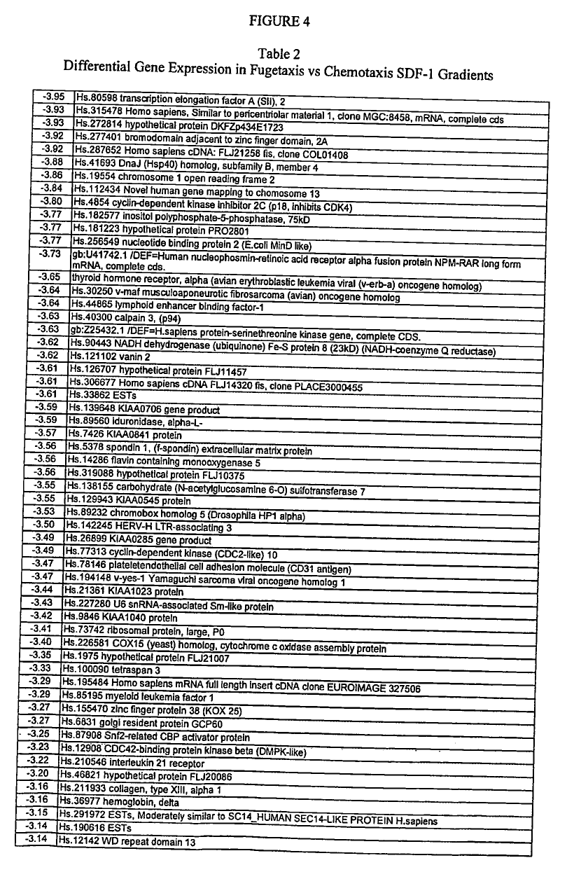

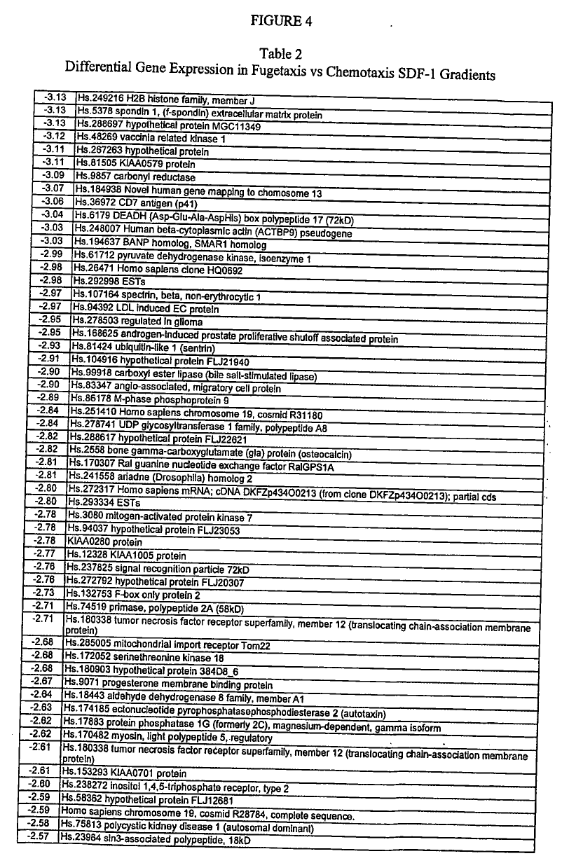

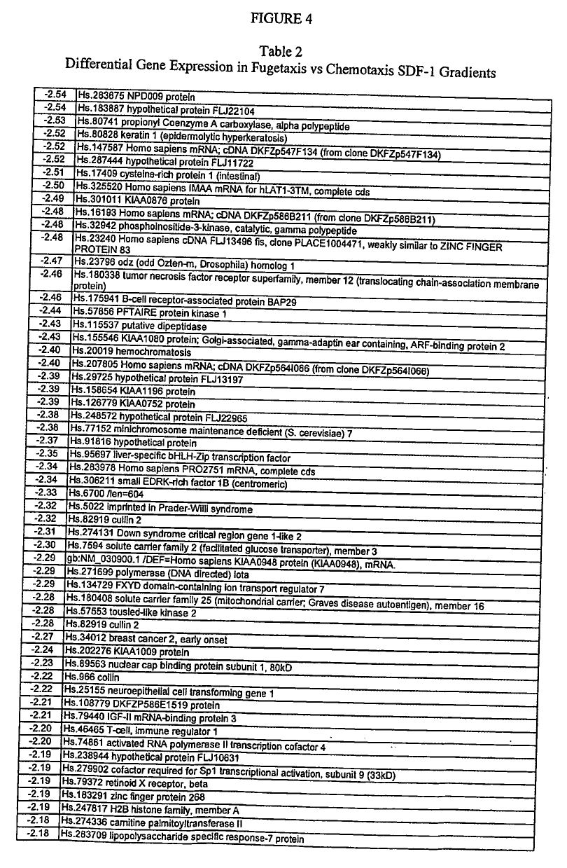

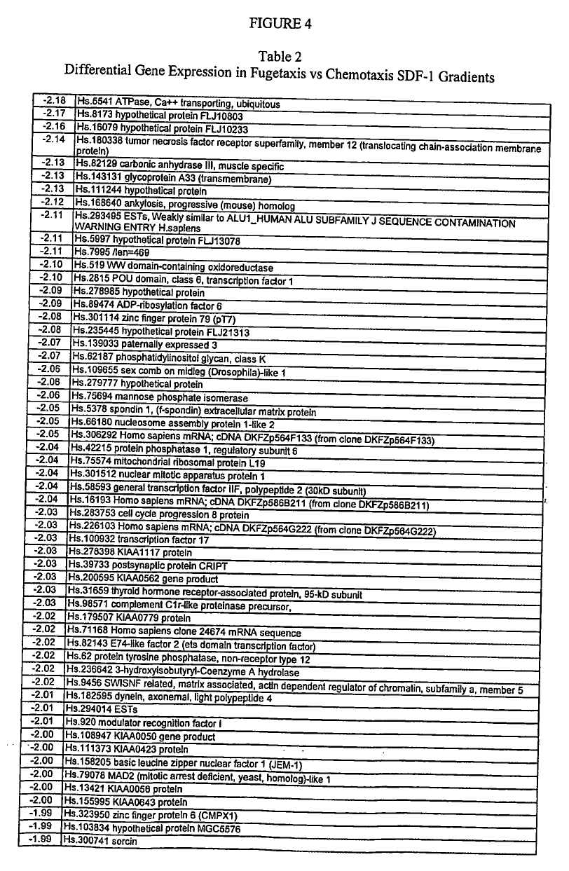

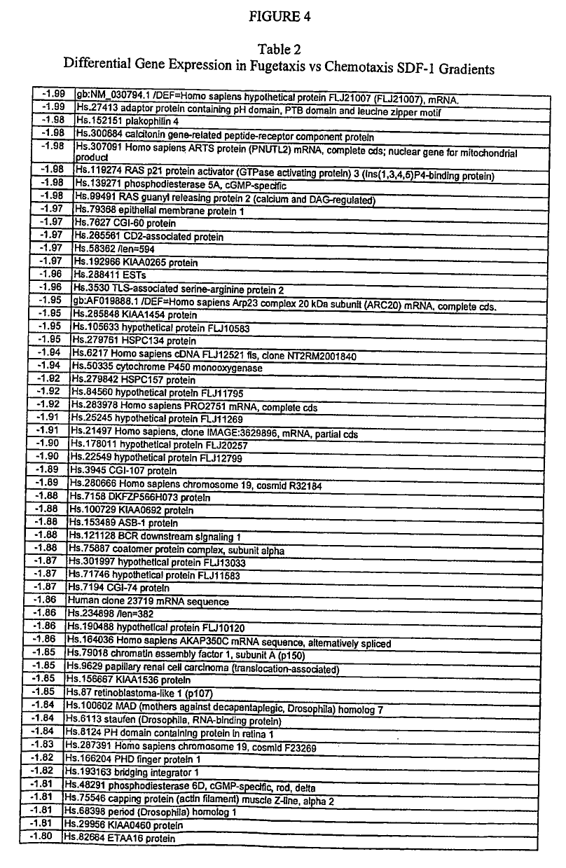

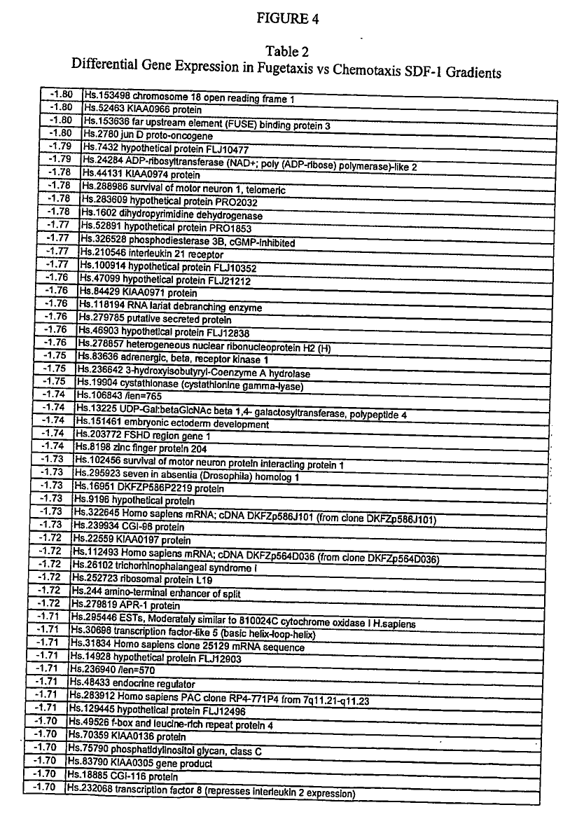

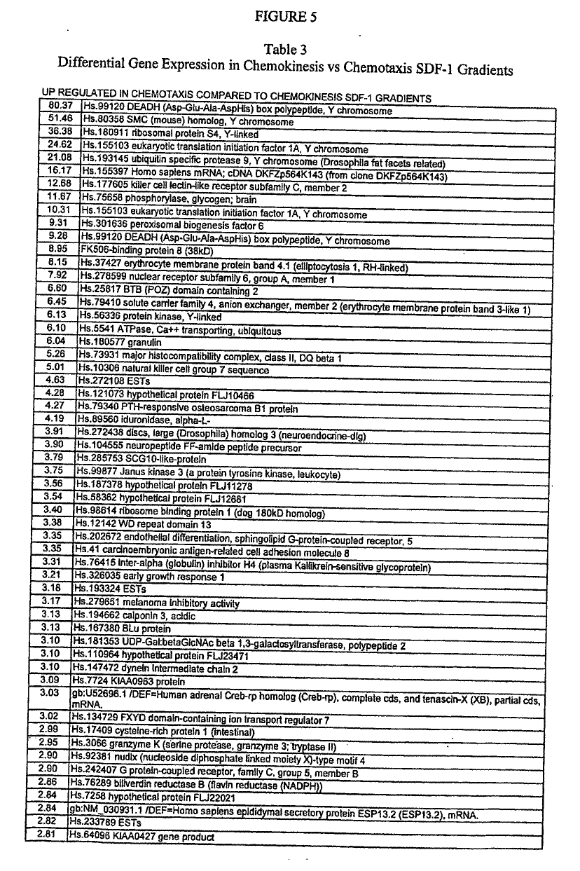

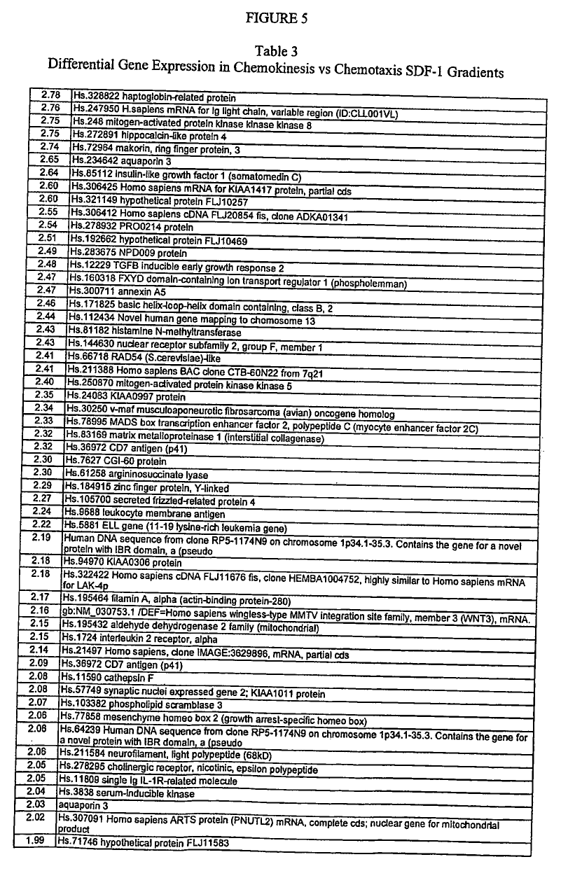

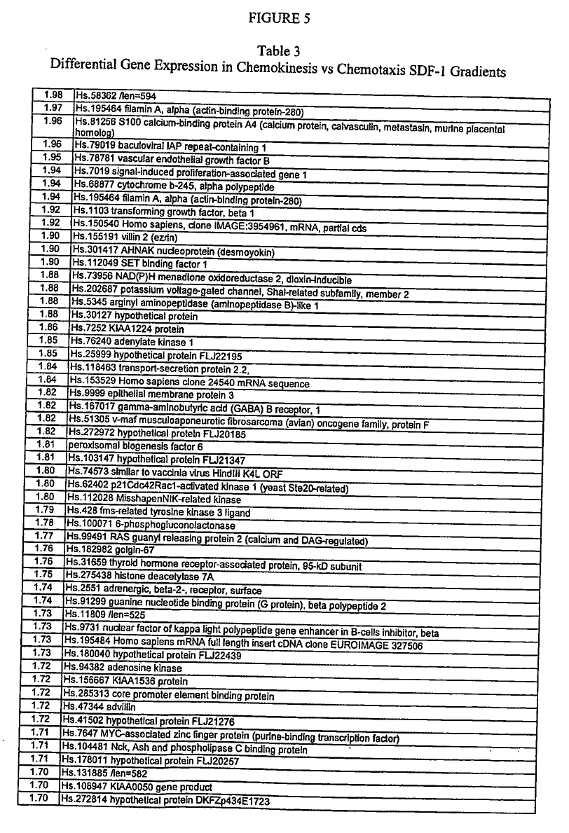

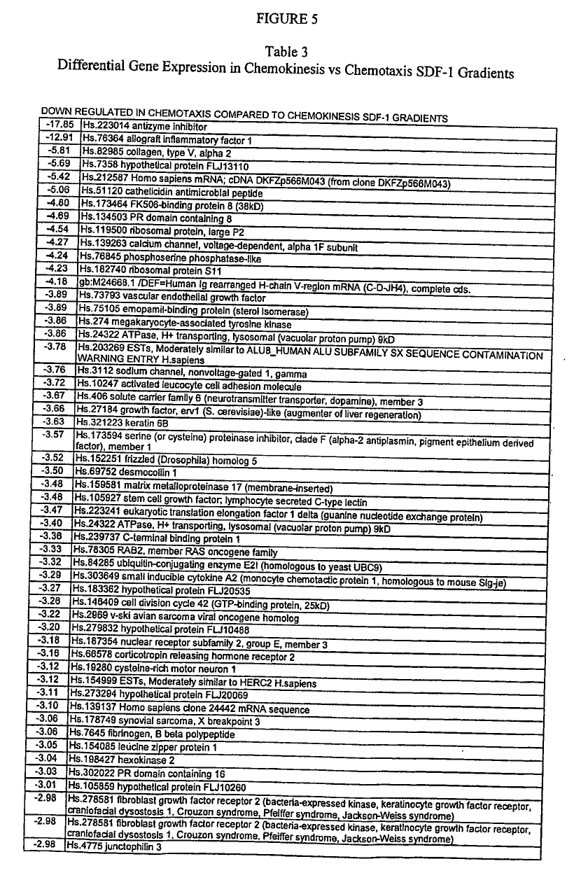

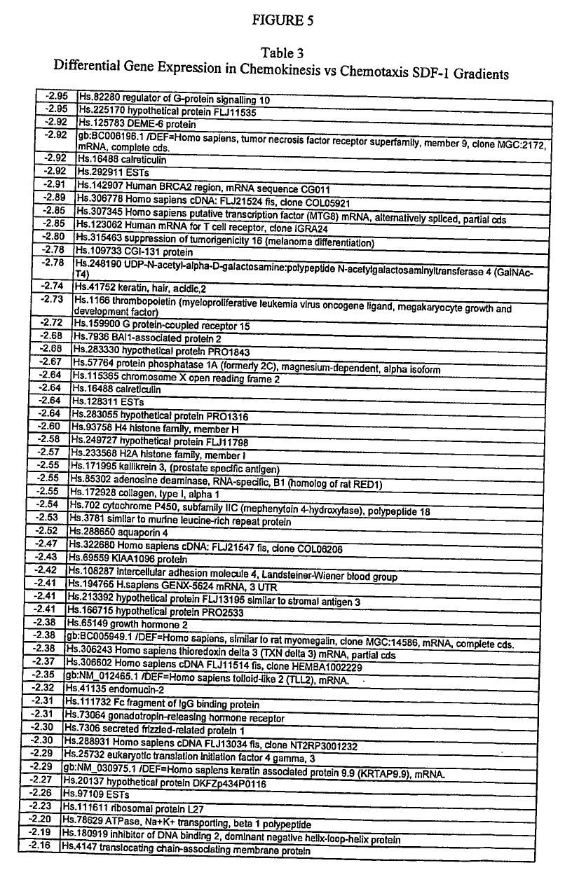

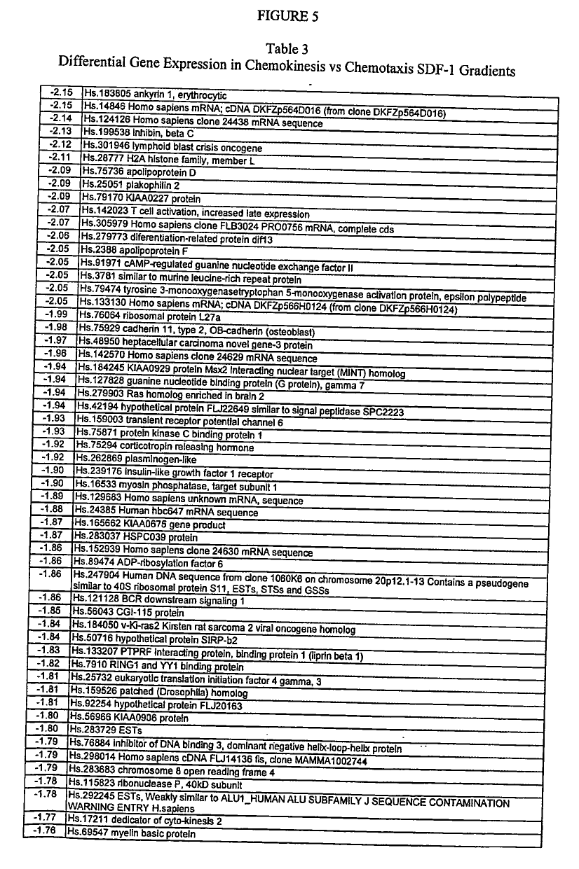

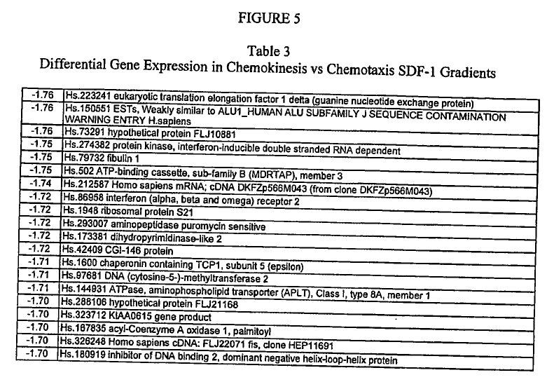

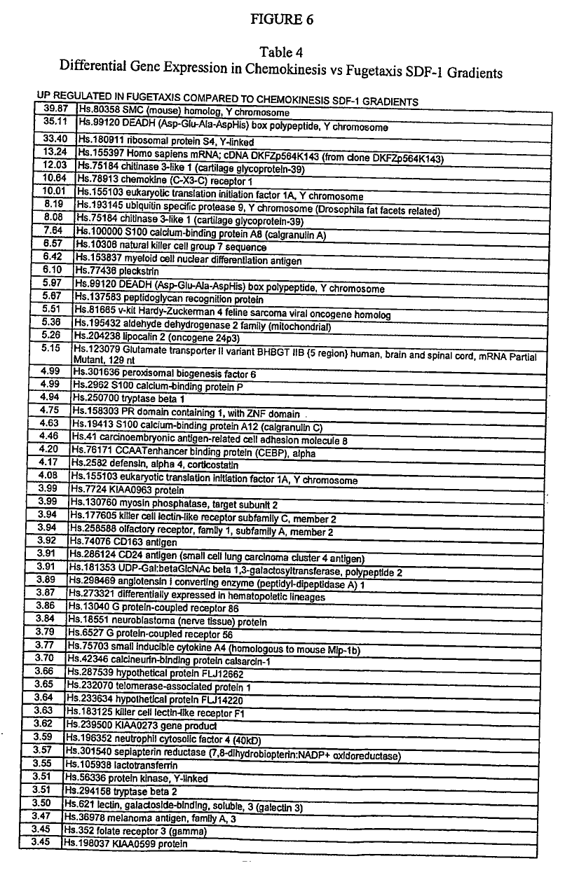

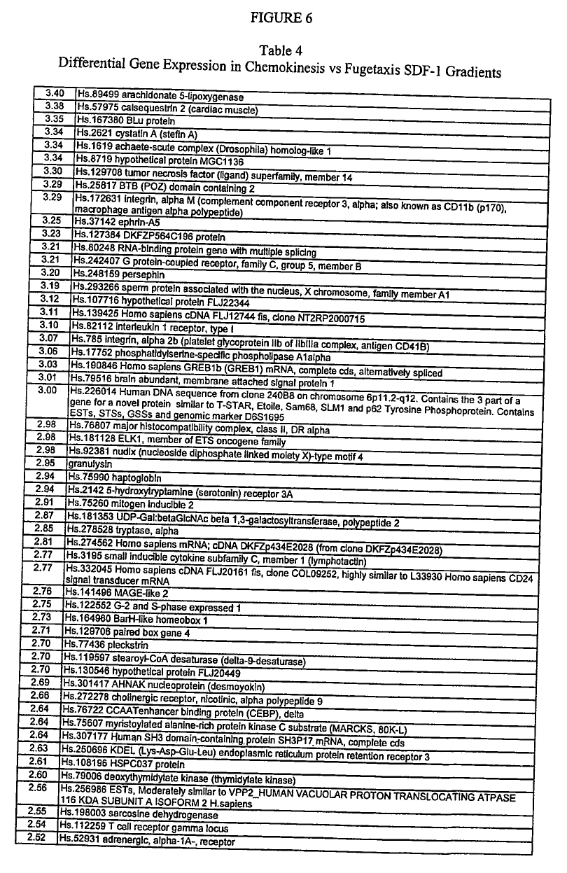

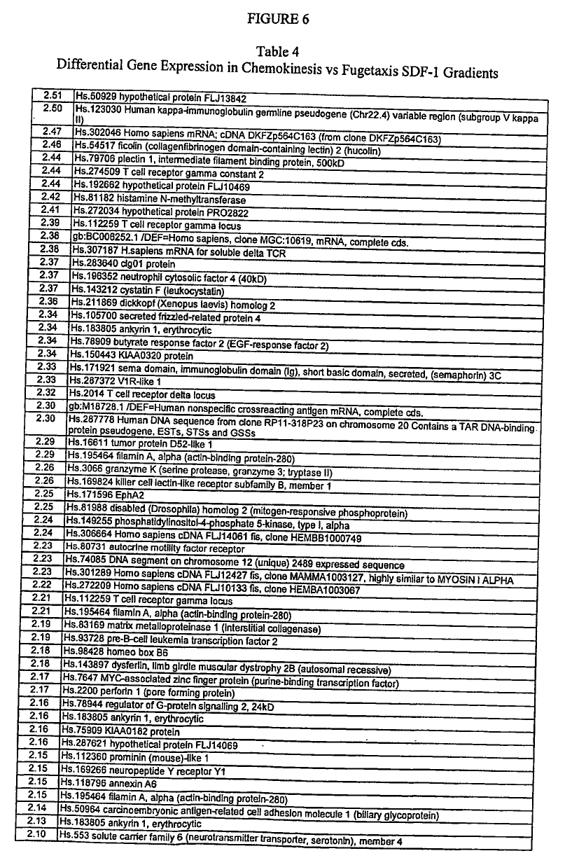

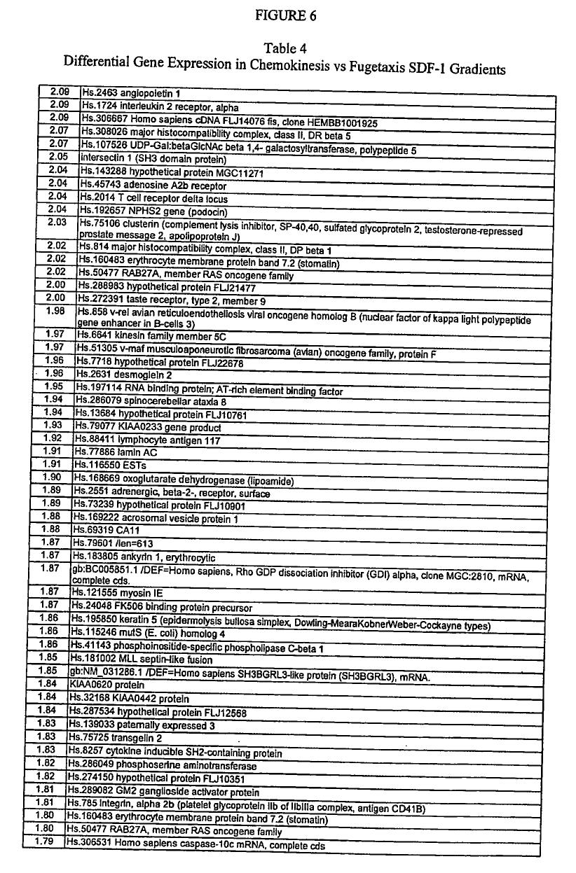

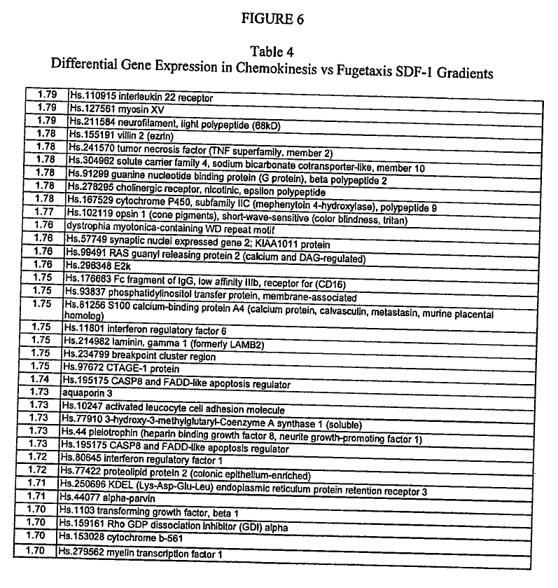

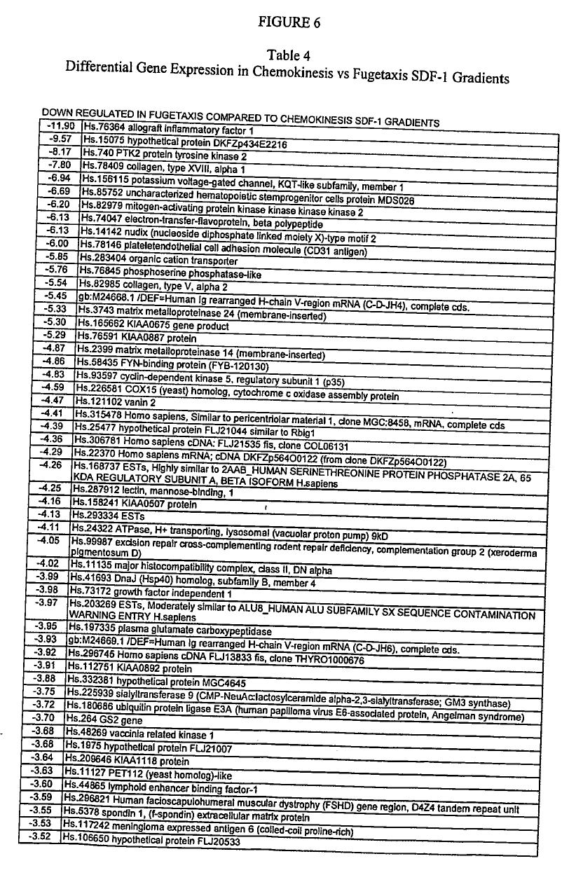

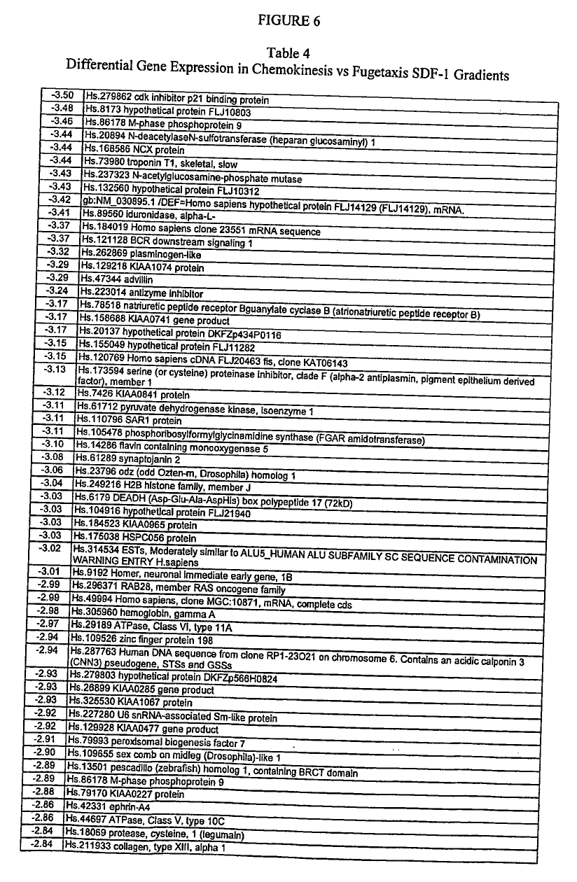

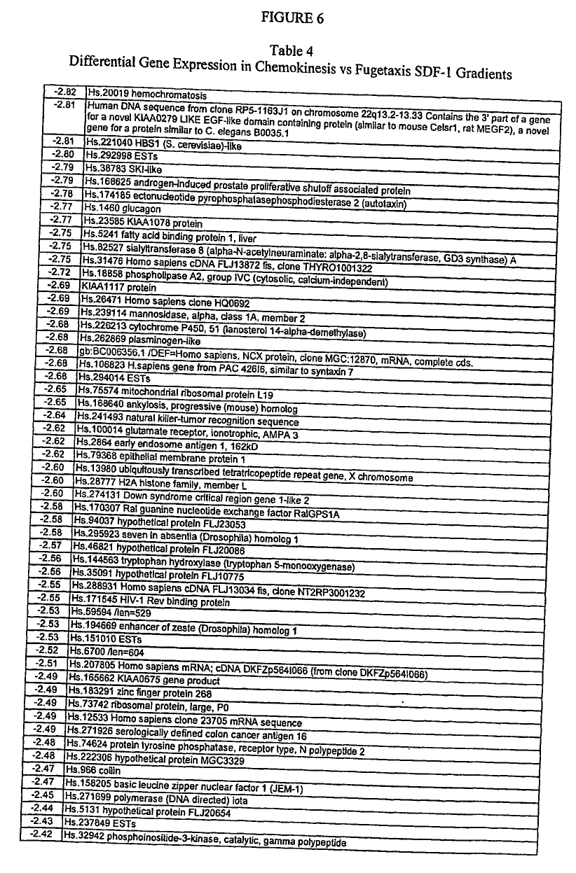

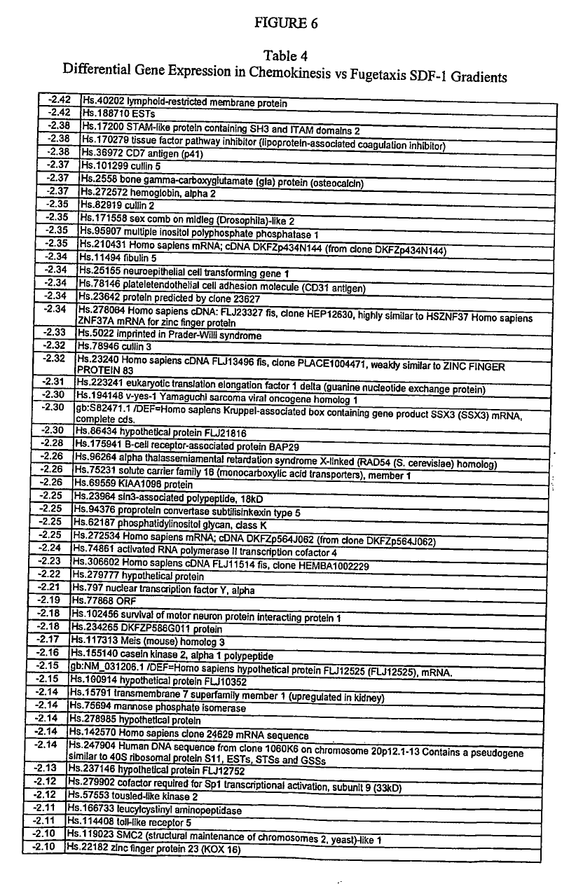

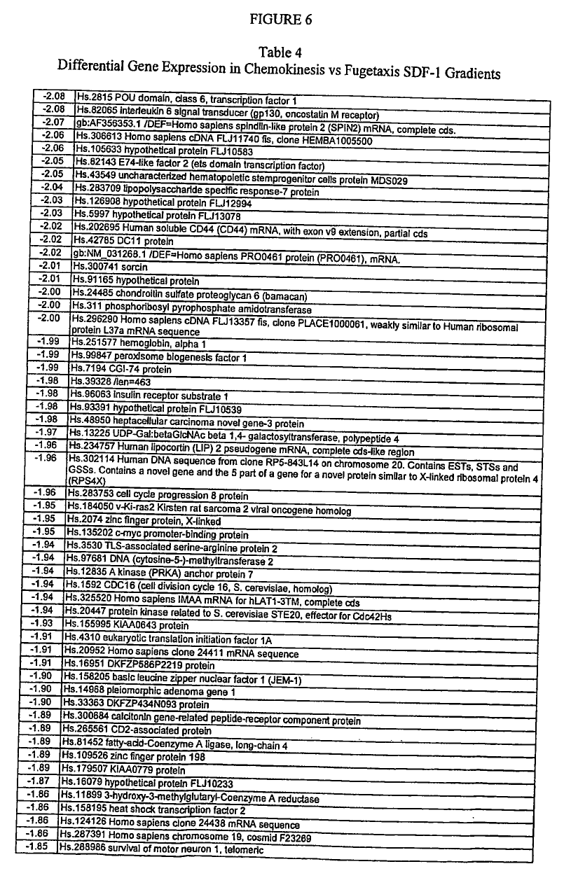

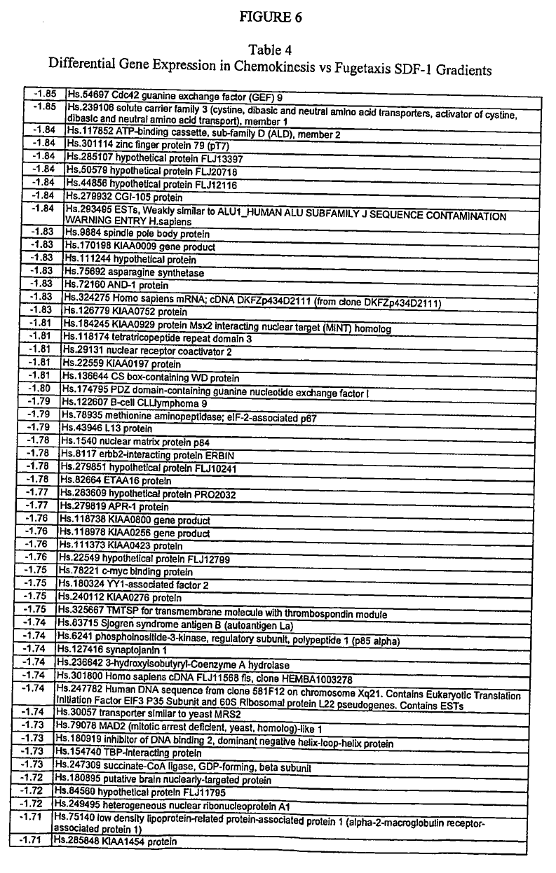

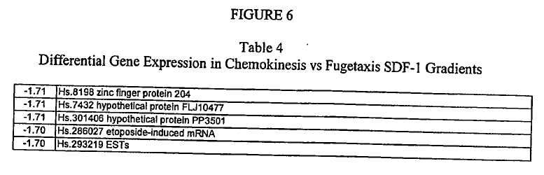

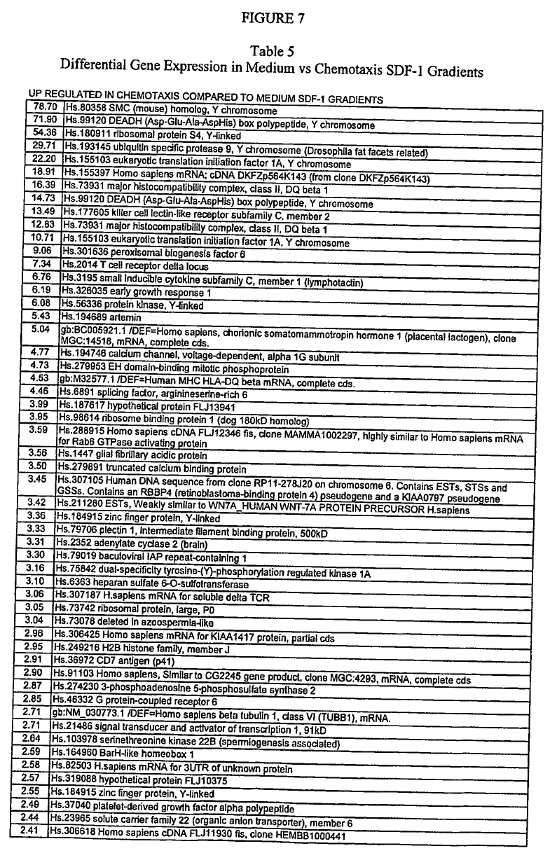

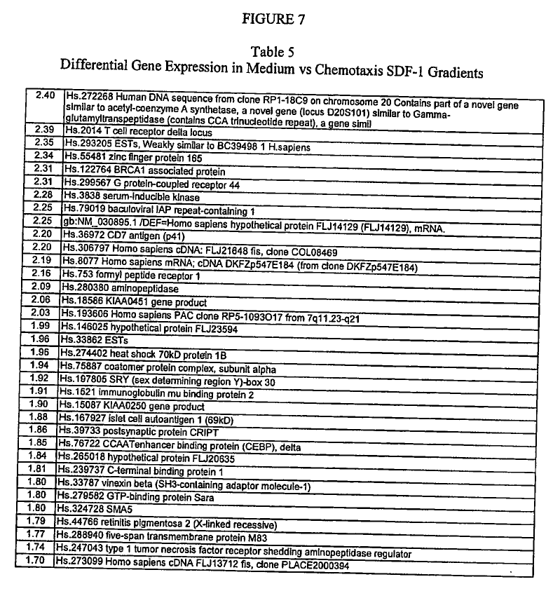

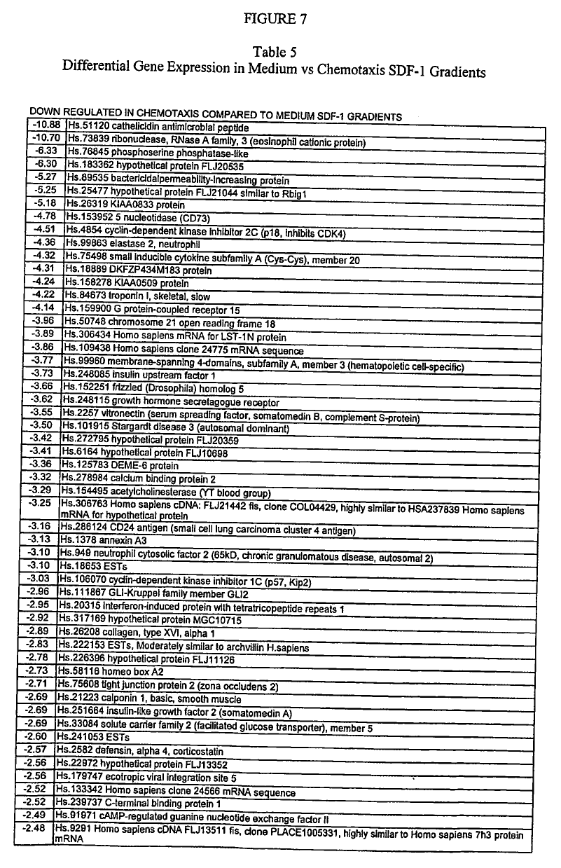

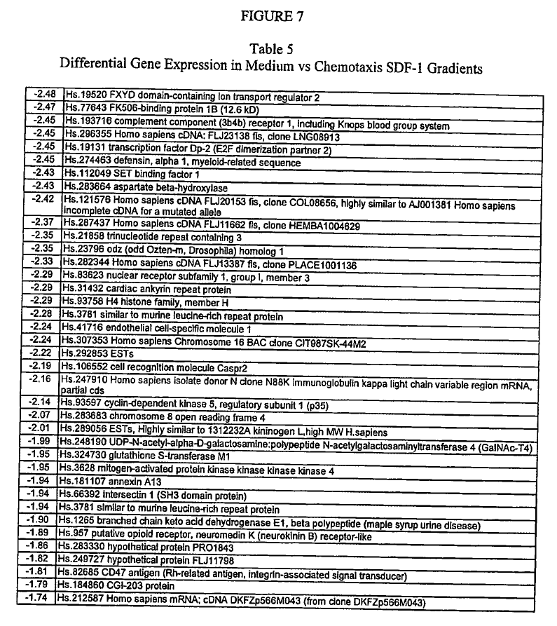

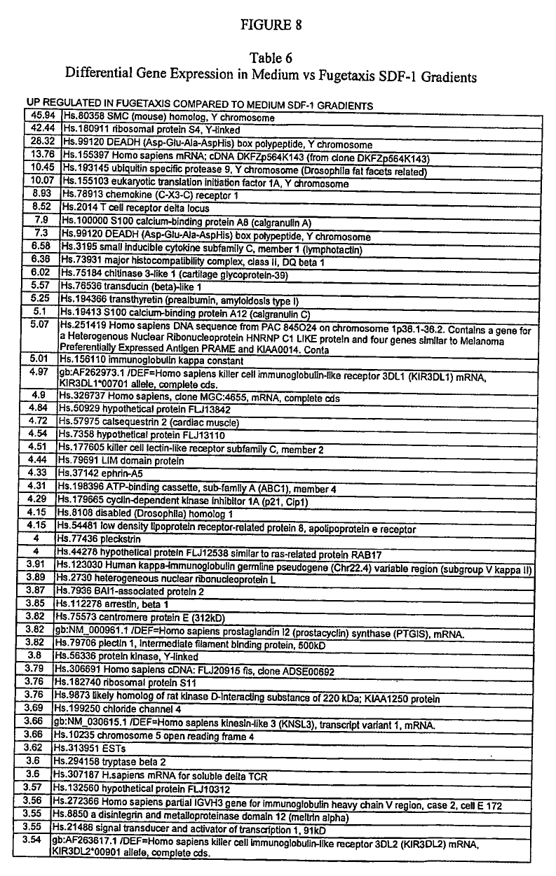

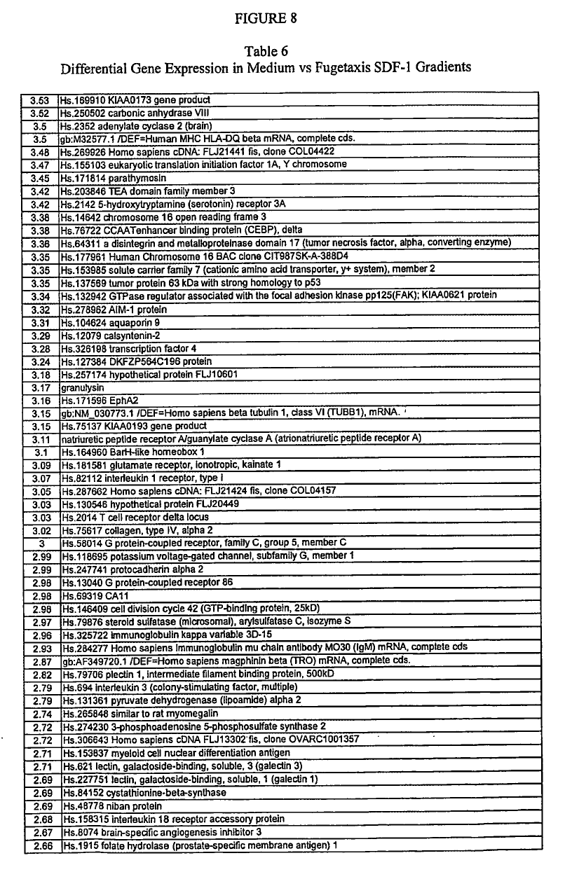

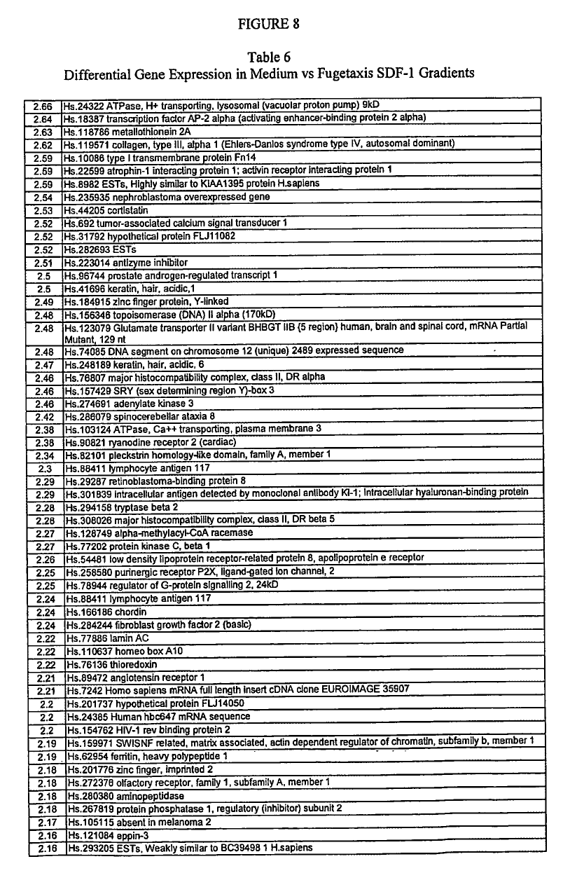

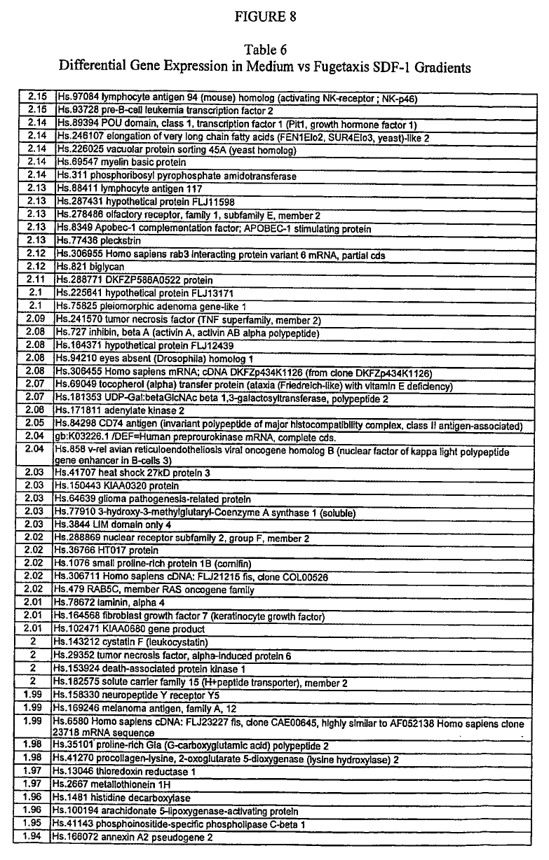

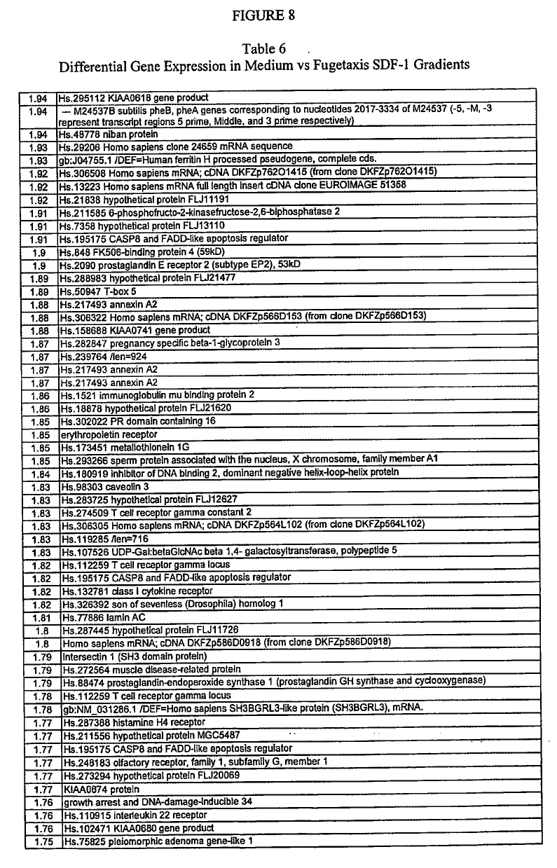

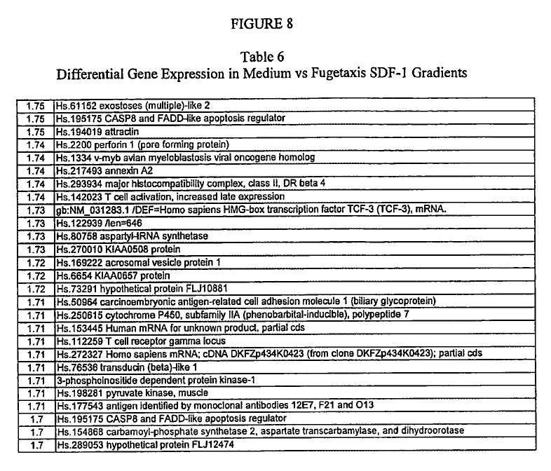

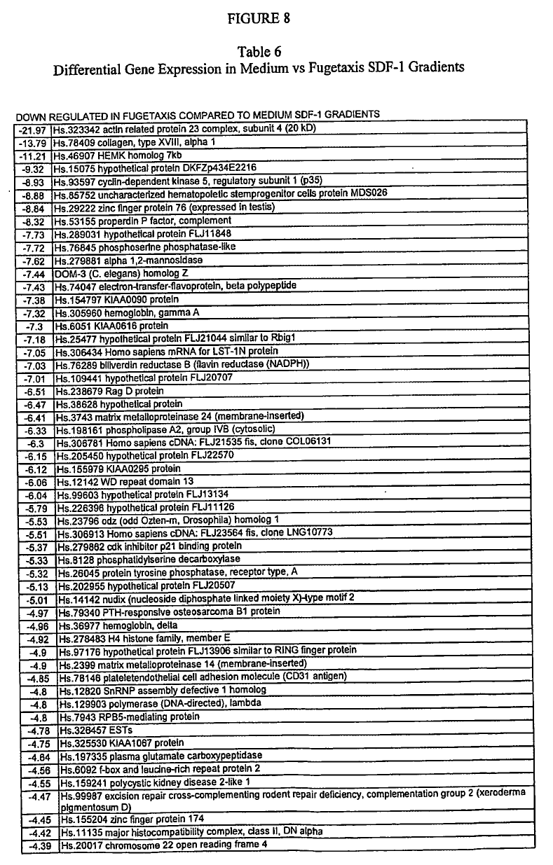

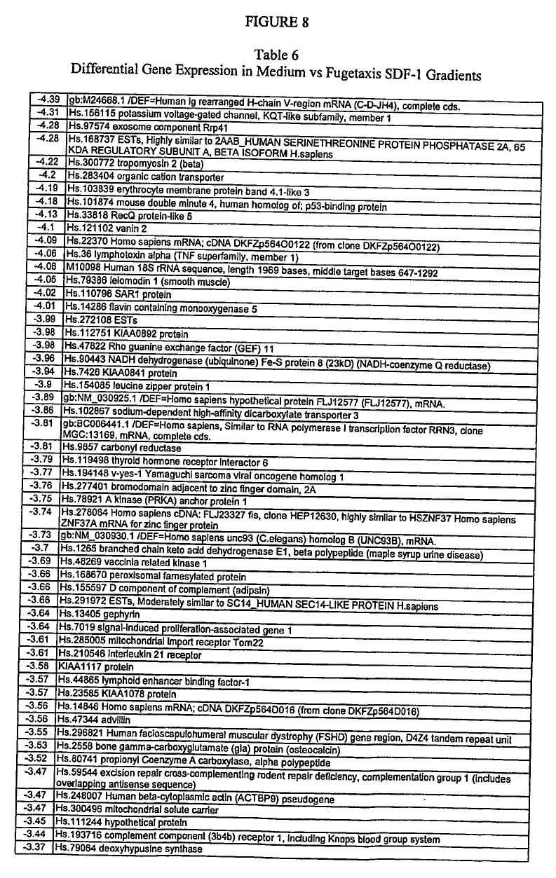

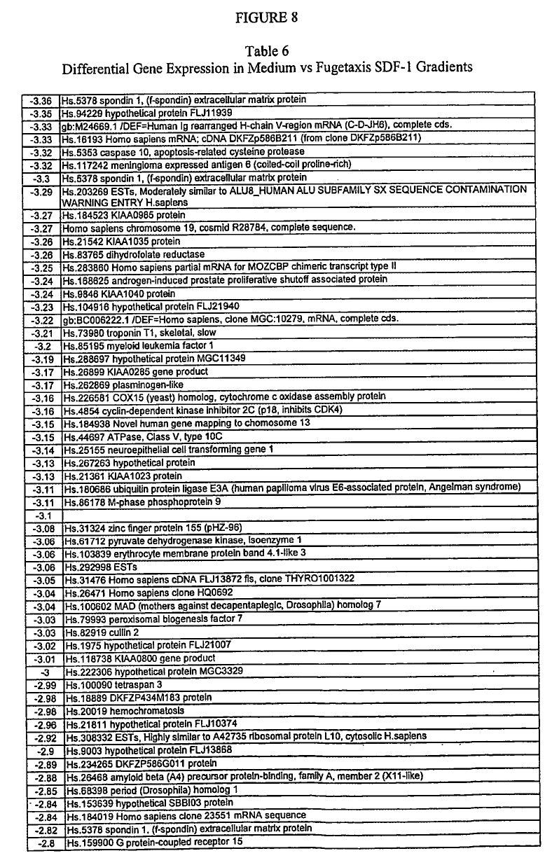

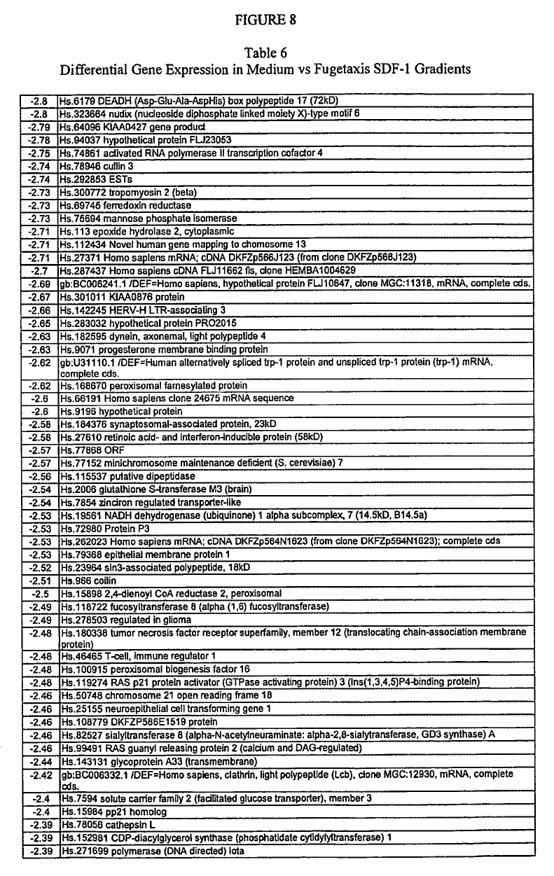

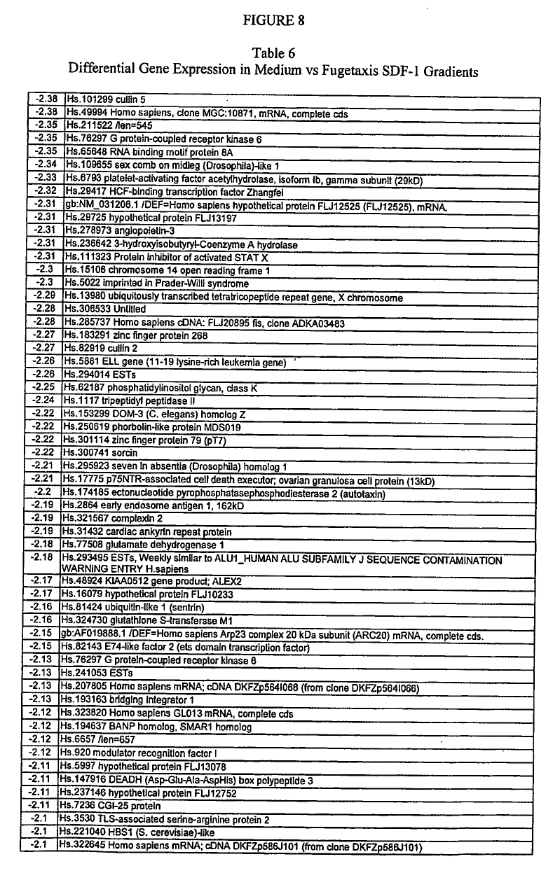

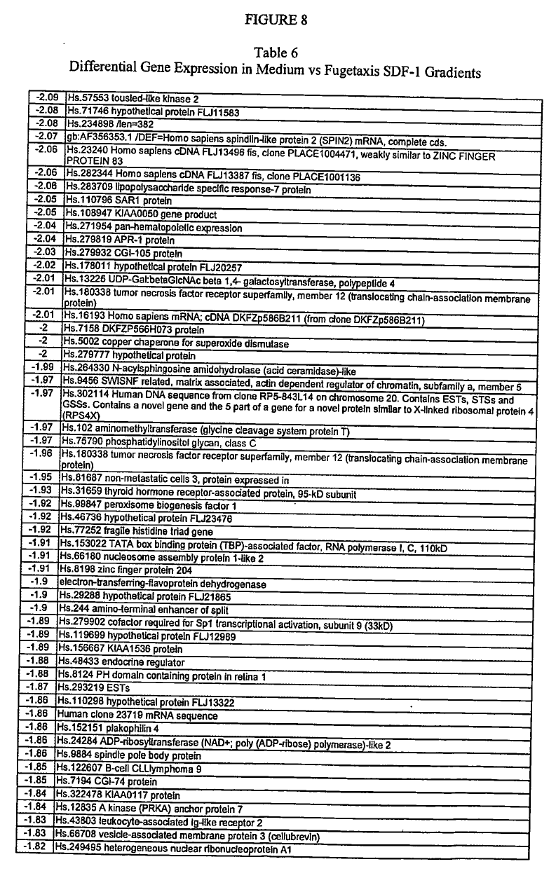

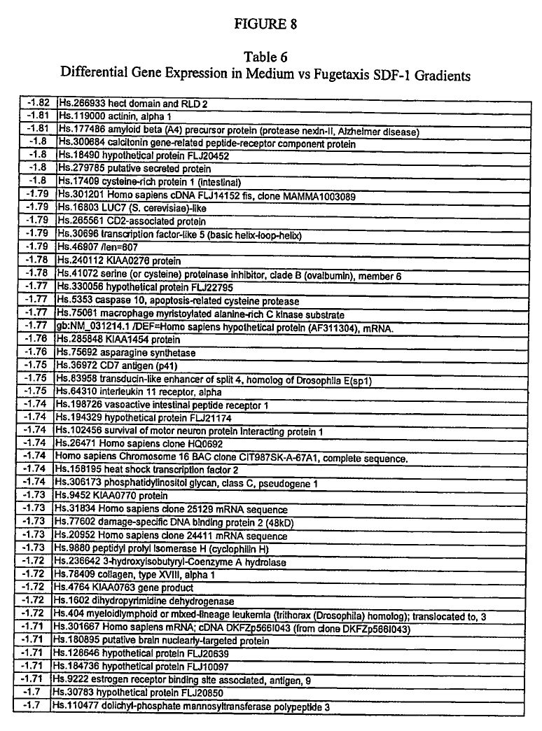

The data are presented in Tables 1-6 (FIGS. 3-8).

Signaling molecules that are upregulated in a uniform gradient of SDF-1 (chemokinetic) gradient of SDF-1 include PTK2 (+6.88) and Regulator of G-protein signaling 10 (+2.53).

Signaling molecules that are downregulated in a uniform gradient of SDF-1 (chemokinetic) gradient of SDF-1 include Phospholipase C, beta 3 (−2.54), RAS p21 protein activator (GAP) 3 (−2.20), Ras guanyl releasing protein 2 (calcium/DAG) (−2.16), G protein-coupled receptor kinase 6 (−2.15), Rho-specific GEF (p114) (−1.70), Protein kinase C substrate 80K-H (−1.70).

Signaling molecules that are upregulated in the presence of a directional (chemotactic and fugetactic) versus neutral (chemokinetic) gradient include Transforming growth factor, beta 1 (1.92 Chemokinetic vs Chemotactic; 1.70 Chemo Fugetactic) and Guanine nucleotide binding protein (1.74 Chemokinetic vs Chemotactic; 1.78 Chemokinetic vs Fugetactic).

Signaling molecules that are downregulated in the presence of a directional (chemotactic and fugetactic) versus neutral (chemokinetic) gradient include Allograft inflammatory factor 1 (−12.9 Chemokinetic vs Chemotactic; −11.9 Chemokinetic vs Fugetactic), Phosphoserine phosphatase-like (−4.24 Chemokinetic vs Chemotactic; -5.76 Chemokinetic vs Fugetactic) BCR downstream signaling 1 (−1.86 Chemokinetic vs Chemotactic; −2.14 Chemokinetic vs Fugetactic) v-Kit-ras2 Kirsten rat sarcoma 2 viral oncogene (−1.84 Chemokinetic vs Chemotactic; −1.95 Chemokinetic vs Fugetactic).

Signaling molecules differentially expressed between a positive (chemotactic) and a negative (fugetactic) gradient of SDF-1.

Signaling molecules that are more highly expressed in a chemotactic gradient of SDF-1 (versus a fugetactic gradient) include PTK2 (focal adhesion kinase) (8.59), MAP kinase kinase kinase kinase 2 (7.30), Guanine nucleotide binding protein (4.95), PT phosphatase receptor (4.20), CDC42-binding protein kinase beta (3.23), Ral GEF (RalGPSIA) (2.81), MAP kinase 7 (2.78), Autotaxin (2.63), Inositol 1,4,5-triphosphate receptor (2.60), Phosphoinositide-3-kinase, gamma (2.48), PT phosphatase, non-receptor (2.02), Ras p21 protein activator (GAP) (1.98), Ras guanyl releasing protein 2 (1.98) and Arp23 complex 20 kDa subunit (1.95). Signaling molecules that are more highly expressed in a fugetactic gradient of SDF-1 (versus a chemotactic gradient) include Cell division cycle 42 (4.93), Ribosomal protein S6 kinase (2.91), BAI1-associated protein 2 (2.84), GTPase regulator associated with FAK (2.59), Protein kinase C, beta 1 (2.16), Phosphoinositide-specific phospholipase C-beta 1 (1.99), Nitric oxide synthase I (1.99), Phosphatidylinositol-4-phosphate 5-kinase (1.82) and MAP kinase kinase kinase kinase 4 (1.72).

Conclusions

This work elucidates the mechanism of bi-directional T cell migration in vitro and in vivo in response to gradients of SDF-1 and shows that the regulation of gene expression associated with the signal transduction pathway for chemotaxis is distinct from that which is associated with fugetaxis. This work forms the basis for identifying potential molecular targets for specific therapeutic agents which could selectively block or enhance the chemotactic or fugetactic responses of T cells to gradients of SDF-1 in vivo.

### EXAMPLE 2

Example 2 describes experiments and findings that demonstrate a new aspect of neutrophil migration in response to the chemokine, Interleukin-8, namely bi-directional movement. Specifically, use of specific non-peptide antagonists of the L-8 receptor, CXCR2, and known inhibitors of chemokine signal transduction reveal that neutrophils can make a directional decision to move up and down an IL-8 gradient and that this decision is dependent on the steepness of the gradient, the absolute concentration of the chemokine that the neutorphil is exposed to, and the level of occupancy of the CXCR2 receptor. Moreover, the directional decision of neutrophils to migrate down a gradient was also found to be differentially sensitive to signal transduction inhibitors as compared to migration up the gradient.

Methods

Primary human T cells were exposed to specific gradients of IL-8 to induce chemotaxis or fugetaxis in vitro and in vivo in microfabricated devices. Intravital microscopy and digital image analysis were used to examine neutrophil bi-directional movement in response to Il-8.

Neutrophil isolation: Human whole blood was obtained from healthy volunteers by venipuncture into tubes containing sodium heparin (Becton Dickinson, San Jose, Calif.). Whole blood was centrifuged for 4 minutes at 2400 rpm and plasma was removed. Resulting pellet was resuspended in Iscove's Modified Dulbecco's Medium (IMDM; Cellgro MediaTech, Herndon, Va.) with 0.5% (w/v) fetal calf serum FCS; Cellgro MediaTech). 25 mL of suspension was layered over 10 mL Lymphocyte Separation Medium (ICN, Irvine, Calif.) and centrifuged for 40 minutes at 1600 rpm at 22° C. Supernatant was aspirated, resulting pellet was resuspended in IMDM with 0.5% (w/v) FCS and 2% (w/v) dextran (Sigma-Aldrich, St. Louis, Mo.), and red blood cells (RBC) were allowed to sediment for 30 minutes at room temperature. Supernatant was transferred into clean tube and centrifuged for 5 minutes at 2000 rpm. Supernatant was aspirated, pellet was mixed with cold ddH₂O for hypotonic lysis of remaining RBCs, and transferred to IMDM with 0.5% (w/v) FCS. Isolated neutrophils were washed and resuspended in IMDM with 0.5% (w/v) FCS, determined to be 95% pure, and 99% viable by Trypan Blue exclusion.

Fabrication and Preparation of Microfluidic Linear Gradient Generator: The microfluidic linear gradient generator was fabricated in poly(dimethylsiloxane) (PDMS; Sylgard 184, Dow Corning, N.Y.) using rapid prototyping and soft lithography as described previously. Briefly, a high resolution printer was used to generate a transparency mask from a computer-aided design image file. The mask was used in contact photolithography with SU-8 photoresist (Microlithography Chemical Co., Newton Mass.) to generate a positive relief of patterned photoresist on a silicon wafer. Replicas with embossed channels were fabricated by curing PDMS prepolymer against the patterned wafer. Inlet and outlet ports for media and cell suspension were bored out of the cured PDMS replica using a sharpened syringe needle. The PDMS replica and glass substrate were placed in an oxygen plasma generator (150 mTorr, 100 W) for 1 minute. Immediately following plasma treatment, the PDMS replica and glass were placed against each other and irreversibly bonded. Polyethylene tubing (Becton Dickenson) was inserted into inlet and outlet ports to make the fluidic connections. Tubing was connected to a PHD 2000 syringe pump (Harvard Apparatus, Holliston, Mass.) to complete the setup. Hemostats were used to control flow during cell loading.

Characterization of Linear Gradient Generator: Verification of gradient formations in the microfluidic device were carried out using solutions of phosphate buffered saline (PBS; Cellgro MediaTech) and fluorescein isothiocynate (FITC; Sigma-Aldrich) as previously described. Verification of gradient formations in the microfluidic device were carried out using solutions of Dulbecco's phosphate buffered saline (DPBS; Cellgro MediaTech) and fluorescein isothiocynate (FITC; Sigma-Aldrich) as previously described. Briefly, PBS and PBS with 100 μM FITC were introduced into the device. Fluorescent micrographs were taken of the stable gradients at various steady flow speeds (0.1, 1, 10, 10 mm/s). Graphs of the fluorescent intensity profile across the migration channel demonstrate generation of temporally and spatially stable linear gradients; profiles at low flow rates are smooth and continuous, while increased flow speed yields stepped gradients as fluid flow becomes more laminar. (Li Jeon et al, Nat Biotech 2002; Li Jeon et al, Langmuir 2000).

Microfluidic Migration Assay and Timelapse Microscopy: Neutrophils (1×10³ cells) were placed uniformly across the migration channel and allowed to migrate under a linear gradient of human Interleukin-8 (72 a.a.; PeproTech, Rocky Hill, N.J.) in IMDM with 0.5% (w/v) FCS flowing at 0.1 mm/sec. Migration was observed in a Nikon Eclipse TE2000-S microscope (Nikon, Japan) through a 10× Plan-Fluor objective (Nikon). Brightfield images were taken every 30 seconds using a C4742-95 Hamamatsu digital camera (Hamamatsu, Japan) controlled by IPLab 3.6.1 (Scanalytics, Fairfax, Va.). Cell movement was always observed at a set point along the migration channel. Gradients were also calibrated at this set point. Migration was quantified for all cells across the gradient.

Construction of Digital Videos for Quantitative Analysis: Digital videos were mad from time-lapse video microscopy file stacks or S-VHS videotapes using a combination of IPLab 3.6.1, Photoshop 6.0 (Adobe Systems, San Jose, Calif.), and Apple QuickTime Pro 5.0 (Apple Computer, Cupertino, Calif.). Migration tracking was carried out using MetaMorph 4.5 (Universal Imaging, Downington, Pa.) object tracking application, which generated tables of Cartesian coordinate data for each tracked cell.

Mathematical Analysis of Cell Migration in Linear Gradient Generator: The angular correlation function, or cosine correlation function, was calculated for each experiment. For experiments with no gradient, the correlation function decayed exponentially with increasing time interval, while the function decayed much slower, potentially by a power law, for experiments with a gradient; in all cases correlation of angles over time was increased as absolute [IL-8] increased. The fact that angular choice is correlated over time allowed us to compare angular frequency distributions as an index of directional migration Cell movement within the linear gradient generator was characterized based on a biased random walk model (Moghe et al, J Immun Methods 1995; Tan et al, J Biomed Mater Res 2000), thus the movement between tracked positions in successive frames of a video can be considered as a vector, with a length and associated angle. Tracking data from MetaMorph was analyzed in Excel (Microsoft, USA) and MATLAB 13 (Mathworks, Inc.) to determine mean squared displacements, coefficients of motility, angular frequencies and correlations, random walk path lengths, and migration velocities. Cell motility was characterized as follows. For each cell, the squared displacement R²(t) was calculated at time interval t,
<R²(t)>=<(x(t₀+t)−x(t₀))²+(y(t₀+t)−y(t₀))²>,

where t₀ is the time at the origin. The origin was shifted along the data set and the displacements were averaged for overlapping time intervals. A global average was performed over all cells in the set to calculate the mean squared displacement. Mathematically modeling cell movement as a correlated, biased random walk, this can be written as
<R²(t)>=2S²P[t−P(1−e^{-t/P})],

where S and P are measures of the rate of movement and persistence time respectively. When time interval t is much greater than persistence time P, the mean squared displacement becomes linearly proportional to t, analogous to Brownian diffusion,
<R²(t)>=2S²Pt=4μt

where μ is the motility coefficient. The slope and intercept of a least squares regression fitted to the linear section of the mean squared displacement give an estimate of μ and P, respectively. Additionally, a “persistence index” (PI) of the motion or mean free path, was calculated as the total displacement of the cell divided by the total distance traveled along the track. The PI is an indicator of turning behavior, with 1 indicating motion in a straight line and 0 indicating no net displacement.

The directional bias of cell motility was quantified as follows. For each cell, histograms of angle frequency show the distribution of angles associated with each displacement vector between successive time intervals of migration. The binning of these histograms can be varied to reduce the stochastic noise associated with a random walk. The x-axes of these histograms are folded around one point to create a circular histogram presenting the angular frequencies in 360°. The angular correlation function (or cosine correlation function) was calculated as:
g(τ)=φ(t).φ(t+τ)>=<cos[φ(t)−φ(t+τ)]>,

where φ(t) is the angle that the displacement vector makes with respect to the direction of the gradient. The decay of this function with increasing time interval indicates the correlation between successive turn angles and is a measure of the directional persistence or memory of the cells. To quantify directional bias with respect to the established gradient, we calculated the “mean chemotropism index” (MCI), which is defined as the net path length traversed by a cell with respect to the direction of the established gradient divided by the total distance traveled and is a measure of the accuracy of orientation.

$$CI = \frac{\sum {l}_{l} cos {φ}_{l}}{\sum {l}_{l}}$$

The index for each cell was calculated and then averaged over the whole population. The average chemotropism index will be 1 if cells are moving directly up the gradient, 0 if there is no preferred orientation, and −1 for migration directly down the gradient.

Signaling Pathway Inhibitors: Cells were treated with pertussis toxin (100 ng/mL; 30 minutes at 37° C.), wortmannin (1 μM, 10 μM; 20 minutes at 37° C., 8-Br-cAMP (1 mM; 15 minutes at room temperature), 8-Br-cGMP (1 mM; 15 minutes at room temperature) (Sigma-Aldrich), or the CXCR2 non-peptide antagonist, SB225002 (1 pM, 100 pM, 1 nM, or 1 μM for 15 minutes at 37° C.; Calbiochem, Calif.). Immediately after treatment cells were seeded in migration channel of the microfluidic device and allowed to migrate as described above.

Intravital Microscopy: Male Sprague Dawley rats (200-300 g) were purchased from Harlan-Olac (Bicester, U.K.). Male rats were prepared for intravital microscopy. Briefly, following sedation with i.m. Hypnorm (fentanyl-fluanisone mixture, 0.1 ml; Janssen-Cilag, High Wycombe, U.K.), animals were anesthetized with i.v. sodium pentobarbitone (30 mg/kg loading dose followed by 30 mg/kg/h; Rhône Mérieux, Harlow, U.K). The animals were maintained at 37° C. on a custom-built heated microscope stage. Following midline abdominal incision, the mesentery adjoining the terminal ileum was carefully arranged over a glass window in the microscope stage and pinned in position. The mesentery was kept warm and moist by continuous application of Tyrode's balanced salt solution (Sigma Aldrich). Mesenteric post-capillary venules (15-40 μm in diameter) were viewed on an upright fixed-stage microscope (Axioskop F S, Carl Zeiss, Welwyn Garden City, U.K.) fitted with water immersion objectives. Images were captured with a digital camera (C5810-01, Hamamatsu Photonics U.K., Enfield, U.K.) for viewing on a monitor (PVM-1453 MD, Sony U.K., Weybridge, U.K.) and storage by videocassette recorder (AG-MD830E, Panasonic U.K., Bracknell, U.K.). As the resolution of intravital microscopy does not allow definitive distinctions to be made between different subpopulations of leukocytes, all responses are quantified in terms of leukocyte behaviour. Hence, rolling leukocytes were defined as those cells moving slower than the flowing erythrocytes, and rolling flux was quantified as the number of rolling cells moving past a fixed point on the venular wall per minute, averaged for 4-5 min. Firmly adherent leukocytes were defined as those that remained stationary for at least 30 s within a 100-μm segment of a venule. Extravasated leukocytes were defined as those in the perivenular tissue adjacent to, but remaining within a distance of 150 μm of a 100-μm length of vessel segment under study. After baseline readings of rolling, adhesion and transmigration were taken; CINC-1 at final concentrations of 10⁻⁹ M, 10⁻M or 10⁻⁷M (Peprotech) was applied topically to the mesenteric tissue in the superfusion buffer. Leukocyte responses within the chosen vessels were quantified for up to 180 minutes, during which the topical application of CINC-1 was maintained. In each animal, multiple vessel segments from appropriate vessels were quantified. Videos of migrating cells were constucted for quantitative and mathematical analysis as described above; at the end of certain in vivo experiments, the mesentery was stained with acridine orange (Sigma Aldrich), a nuclear dye, scanned with a 488 nm laser line generated from an Argon laser, and observed by confocal microscopy (LSM5 PASCAL, Axioskop II FS, Carl Zeiss) to verify that migrating cells were neutrophils.

Mathematical Modeling of Continuous Gradients in vivo: The chemokine concentration profile in the mesentery at steady state was predicted using a novel in vivo model based on classical diffusion equations applied on a spherical model of the postcapillary venule, and the assumption that the receptor-dependent transport of the chemokine by the endothelial cells is the main mechanism for generating the gradient the vicinity of postcapillary venules. The steady state solution was calculated for the concentration gradient around a sphere in a homogenous medium, with the two boundary conditions: 1) the concentration far from the sphere is constant, and 2) the chemokine flux across the surface of the sphere also constant. Other mechanisms of chemokine transport out of the tissue were considered less significant due to the low lipid solubility of CINC-1 and IL-8 and the presence of tight intercellular junctions between endothelial cells in the absence of vasoactive signals (Middleton et al, Cell 1997). Thus, the steady state concentration C at distance r from the capillary wall was calculated as:

$$C ( r ) = {C}_{0} - \frac{{F}_{0} {a}^{2}}{D ( r + a )} ,$$

where, C₀ is the chemokine concentration in the perfusion solution (either 10 or 100 nM), a the vessel radius (12.5 μm), F₀ the rate of chemokine uptake, and D the diffusion coefficient. The rate of chemokine uptake by the endothelial cells was estimated in the range of 1,000 to 10,000 molecules/cell/min by comparison with endocytosis rates for other proteins (Schwartz, Annu Rev Immunol 1990). A value of 0.6×10⁻⁷ cm²/s for diffusion coefficient of the CINC-1 (MW 7,800) in the mesentery was interpolated from the diffusion coefficient of albumin (MW 66,000) determined experimentally in similar tissues (Parameswaran et al, Microcirculation 1999).

Results

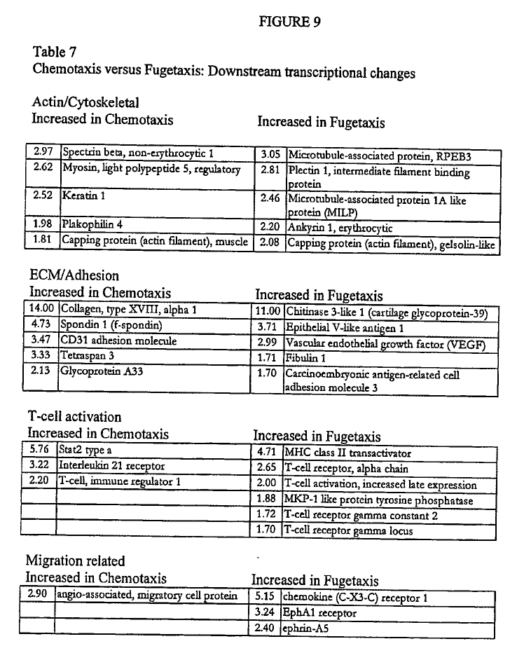

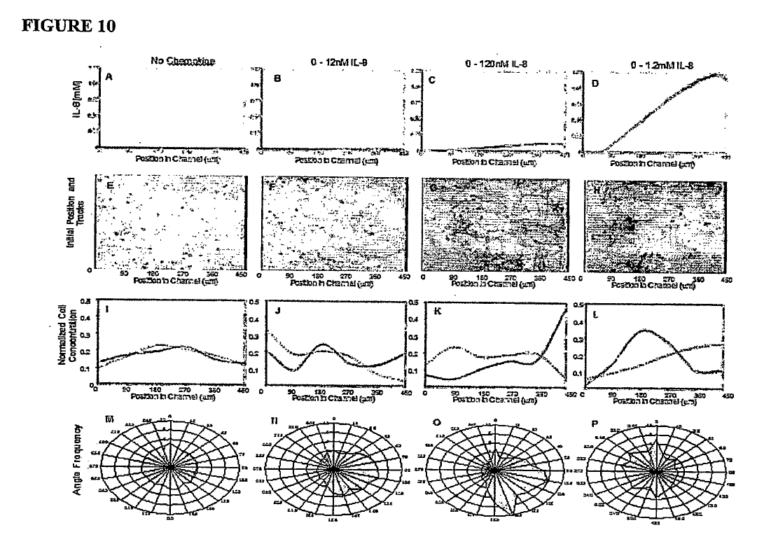

In order to examine whether neutrophils were capable of bi-directional migration continuous gradients of IL-8 of varying steepness in microfabricated devices were established as previously described (Li Jeon, N., et al., (2002) Nat Biotechnol. 20(8):826-30). Previous work with microfabricated devices demonstrated robust chemotaxis of primary human neutrophils in gradients of recombinant human IL-8 between 0 and 50 nM and 0 and 100 nM (Li Jeon, N., et al., (2002) Nat. Biotechnol. 20(8):826-30). Since it had been previously demonstrated that T-cell undergoes fugetaxis at higher concentrations of the chemokine, SDF-1, gradients from 0 to 12 nM, 0 to 12 μM, 0 to 1.2 μM and 0 to 2.4 μM for IL-8 were further examined. Each gradient was initially calibrated and characterized as shown in FIGS. 10A through D and as previously described (Li Jeon, N., et al., (2002) Nat Biotechnol. 20(8):826-30). The differential concentration of chemokine across the migration channel ranged between 0.0267 nM per micron to 5.34 nM per micron or the equivalent of a difference in concentration of the chemokine of 0.267 nM or 50.34 nM across the length of a 10 micron long neutrophil. Neutrophils were also exposed to control conditions including no chemokine or uniform concentrations of IL-8 of 12 nM, 120 nM or 1.2 μM in the migration channel. Human neutroplils were loaded into the device and their migration tracked and quantitated using MetaMorph software in conjunction with MatLab software, respectively (FIGS. 10E through H). The initial and final density of cells across the migration channel was plotted for each of the conditions and the angular frequency of all directional movements determined for each cell using MetaMorph (FIGS. 10I through L). Cells exposed to no chemokine or chemokine at a uniform concentration across the migration channel underwent chemokinesis characterized by angular frequencies in all directions. In contrast, cells placed in gradients between 0 and 12 nM and 0 and 120 nM predominantly demonstrated chemotaxis with predominant angular frequencies occurring towards the peak concentration of the chemokine in the gradient (FIGS. 10M through P). Surprisingly, when cells were exposed to the steepest chemokine gradient of 0 to 1.2 migratory behaviors were more complex. Cells in the lower third of the gradient chemotaxed towards higher levels of the chemokine whereas cells originating in the upper third of the gradient underwent fugetaxis down the gradient and away from the peak concentration of chemokine. Cells initially commencing at a position in the central third of the gradient underwent chemokinesis. The cell density across the migration channel prior to and after neutrophil migration reflects a redistribution of randomly arranged cells to the central third of this gradient (FIG. 10L). In addition, the angular frequency distribution for this gradient reflects a predominant movement away from the chemokine in this gradient (FIG. 10P). Cells exposed to the steepest chemokine gradient studied, (0 to 2.4 μM) underwent chemokinesis regardless of their position within the gradient (data not shown). In this way, the robust bi-directional neutrophil migration within a steep and temporally and spatially stable gradient of IL-8 was observed.

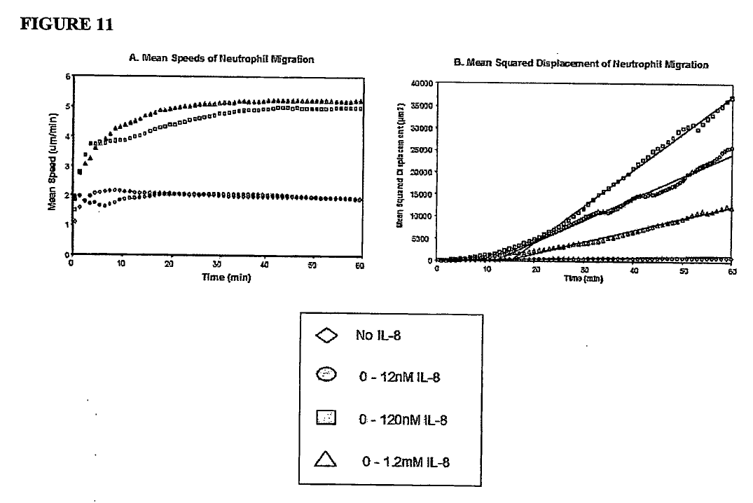

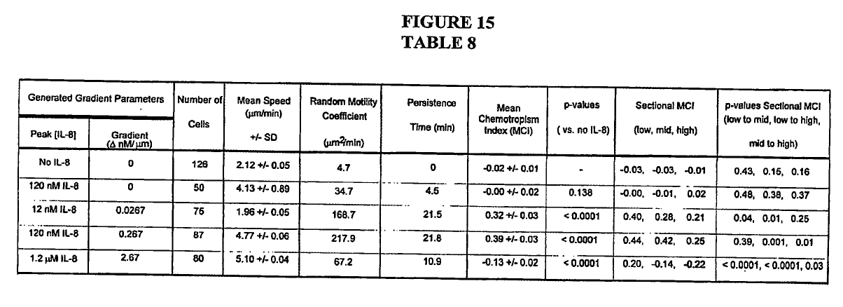

Further, videos of cells migrating in IL-8 gradients were analysed using MetaMorph software and each position of each cell in each frame was defined by its Cartesian coordinates within that frame. It was therefore possible to examine quantitative parameters which describe each cells migratory path. A random walk mode was used to quantitate cell migration, and the previously defined parameters of mean speed, random motility coefficient and persistence time to measure how “diffusive” or “ballistic” cell migration is and mean chemotropism index to measure the directionality of movement towards or away from a chemokine were used. Mean velocity and mean squared displacement for cells migrating in the absence of a chemokine or within gradients in which chemotaxis (0 to 12 nM and 0 to 120 nM) or fugetaxis (0 to 1.2 μM) is seen predominantly (FIGS. 11A and 11B). Measurement of mean velocity demonstrates that cells undergoing chemotaxis in the 0 to 120 nM gradient or fugetaxis in the 0 to 1.2 μM gradient migrate at similar speeds. Mean squared displacement reflects the directional bias of the cells random walk. Chemotaxing and fugetaxing cells demonstrate an exponentially increasing directional bias as they migrate in the 0 to 120 nM and 0 to 1.2 μM gradients, respectively. The gradient of the linear section of the mean squared displacement plot for cells migrating in each experimental and control condition defines the random motility coefficient for cell migration (FIG. 15, Table 8). Random motility coefficients are significantly higher in cells undergoing directional migration in the 0 to 120 nM and 0 to 1.2 gradient than in the presence of a uniform concentration of IL-8 of 120 nM in which chemokinesis predominates. The y-intercept of the linear segment of the mean squared displacement plot indicates the persistence time which is a measurement of how “ballistic” cell movement is (FIG. 15, Table 8). The persistence time for cells migrating in linear gradients of varying steepness are greater than those for cells presented with no chemokine or a uniform concentration of chemokine. Persistence times for cell movement in the IL-8 gradient in which chemotaxis (21.5 minutes) or fugetaxis (10.9 minutes) are seen predominantly are higher than those seen for cells undergoing chemokinesis in the absence of a gradient (0 minutes) or a uniform concentration of IL-8 (4.5 minutes). Chemotaxis and fugetaxis up or down a defined IL-8 gradient approach “ballistic” movement whereas cell movement in the absence of a chemokine gradient is more “diffusive”.

The analysis of cell displacement within a random walk model of cell migration does not measure the directionality of movement towards or away from a chemokine. In addition, treating all cells equally within a gradient assumes that all cells behave in the same way in the same gradient. Since it had been identified that cells can migrate up or down a gradient in a manner that is dependent on their precise position within the gradient, the measurement of mean chemotropism index (MCI) was utilized to define the directionality of movement up (positive values) or down (negative values) a gradient and analysed cell movement three arbitrary sectors of each gradient (FIG. 15, Table 8). Cells exposed to uniform concentrations of chemokine at 120 nM or no chemokine had MCI values of −0.02 +/−0.01 and 0.00+/−0.02 respectively. Cells undergoing chemotaxis in gradients between 0 and 12 nM and 0 and 120 nM demonstrated MCIs of +0.32 and +0.39 respectively. In contrast, cells exposed to the steeper gradient of 0 to 1.2 μM demonstrated a negative MCI of −0.13 supporting the view that the predominant movement of cells in the gradient was away from the peak concentration of IL-8. Cells migrating in the steepest 0 to 2.4 μM gradient exhibited chemokinesis. In order to further analyse the effect of the influence of both gradient steepness and absolute concentration of the chemokine gradient each gradient was divided into three equal segments and cell populations, and commencing movement in each segment were then analysed separately. Cells migrating in all sectors of the 0 to 12 nM and 0 to 120 nM gradient reveal positive MCIs of between +0.21 and +0.44. Whereas, cells migrating in the lower segment of the 0 to 10.2 μM gradient had a mean sectional MCI of +0.2, cells in the middle third and upper third of the gradient have negative MCIs of −0.14 and −0.22 respectively. These quantitative data which examine both the bias and direction of the random walk confirm the finding of bi-directional neutrophil migration. In addition, these quantitative data confirm that the directional decision of a cell to move up or down a gradient is determined by both the steepness of the gradient and the absolute concentration of the chemokine that it is exposed to within the gradient.

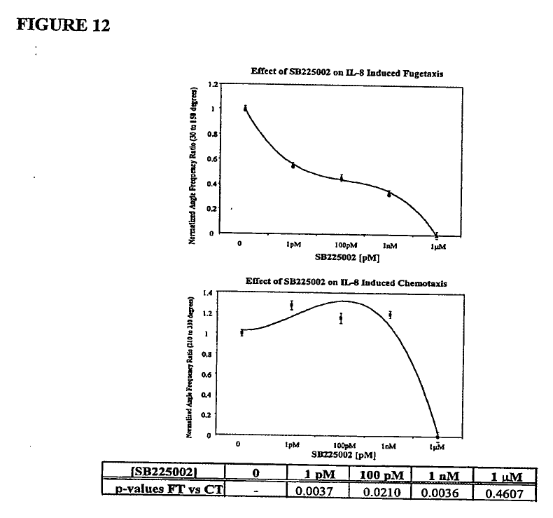

Since receptor occupancy is known to play a role in directional decision making and gradient sensing in the context of chemotaxing eukaryotic cells, it was postulated that chemokine receptor occupancy by a chemokine might also play a critical role in the decision of a cell to move up or down a chemokine gradient. Thus, a SB25002, the specific non-peptide antagonist of the IL-8 receptor, CXCR2 was utilized to examine this postulate. Neutrophils were pretreated with SB225002 at concentrations between 1 pM and 1 μM and then exposed to 0 to 1.2 μM gradients of IL-8 in microfabricated devices as described above. Videos of cell migration were analysed using MetaMorph and MathLab software to generate normalized angular frequencies determined for cells migrating in each of the three sectors of the gradient. The absence of inhibitor generates a normalized angular frequency of 1.0 whereas inhibition of fugetactic or chemotactic angular frequencies results in a normalized frequency of <1.0 and augmentation of either directional response results in a value greater than 1. This analysis allows to precisely quantitate the effect of a given concentration of inhibitor on the directional decision of the cell to move up or down a gradient. The lowest concentrations of SB225002 (1 pM and 100 pM) lead to significant inhibition (p=0.0037 and 0.0210) of fugetaxis whereas chemotaxis was infact augmented under these conditions (FIG. 12). Gradually increasing concentrations of SB225002 ultimately inhibited both fugetaxis and chemotaxis. These data indicate that receptor occupancy plays a significant role in determining the directional decision of a cell to move up or down a steep IL-8 gradient. Furthermore, although IL-8 binds to both CXCR2 and CXCR1 on the cell surface, of the human neutrophil bi-directional signaling was evidently critically dependent on CXCR2.

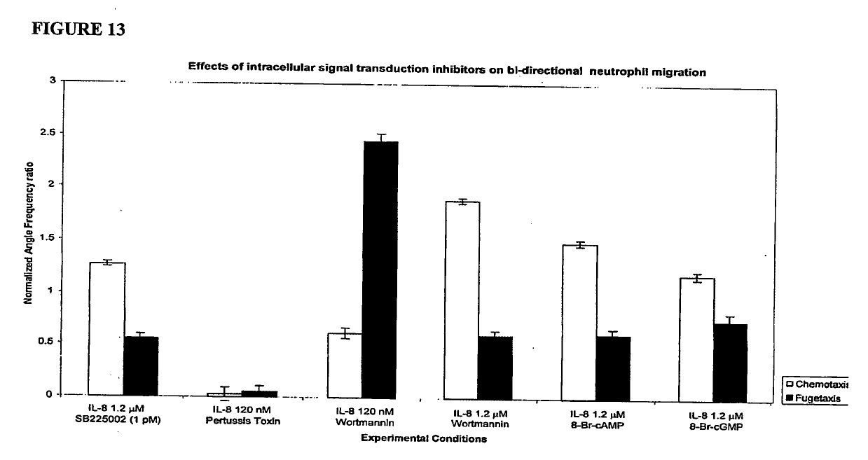

It had been previously shown that the signaling pathway for chemotaxis is distinct from that for chemorepulsion or fugetaxis. It is known that T-cell fugetaxis in response to SDF-1 in standard transmigration assays was differentially more sensitive to inhibition by the intracytoplasmic cyclic nucleotide agonist 8-Br-cAMP than chemotaxis. Furthermore, it had been demonstrated that T-cell chemotaxis was differentially more sensitive to inhibition by the tyrosine kinase inhibitor, genistein, than was fugetaxis. The study of neutrophil migration in microfabricated devices allows to examine precisely the effects of these inhibitors on quantitative parameters of cell migration including the directional bias of cells in the context of precisely defined and stable chemokine gradients. Primary human neutrophils were pretreated with known inhibitors of the chemokine signal transduction pathway including pertussis toxin, wortmannin, genistein, 8-Br-cAMP and 8-Br-cGMP and then exposed to IL-8 gradients in which chemotaxis and fugetaxis were seen. The effect of the inhibitor on directional migration towards or away from the chemokine was quantitated by determining the directional motility index of cells migrating in the context of these gradients. Movement vector angles corresponding to movement up the gradient (30 to 150 degrees—see FIG. 13) were defined as chemotactic and measured movement vector angles corresponding to movement down the gradient (210 to 330 degrees—see figure) were defined as fugetactic. The directional choice of cells to move up or down a chemokine gradient were therefore compared in the presence and the absence of an inhibitor. Active movement with selective inhibition of directional sensing is manifest as an inverse relationship in distribution of angular frequencies between fugetactic and chemotactic sectors; if fugetaxis is inhibited (<1) chemotaxis will be augmented above normal (>1). Abrogation of directional sensing is manifest as a decrease of angular frequency distributions in both sectors towards zero. In this way it was demonstrated that both neutrophil chemotaxis and fugetaxis was significantly inhibited by pertussis toxin (p=0.007 and p=0.003 respectively). 8-Br-cAMP also selectively inhibited fugetaxis (p=4.6×10⁻⁶) while the same concentration of this intracytoplasmic nucleotide agonist augmented chemotaxis (p=0.0008). Wortmannin pretreatment of cells prior to placement in the 0 to 120 nM or 0 to 1.2 pM gradient generated more complex results than expected. Wortmannin significantly inhibited chemotaxis (p=0.0020) and augmenting fugetaxis (p=<0.0001) in the 0 to 120 nM gradient and in contrast to this significantly augmenting chemotaxis (p<0.0001) and inhibiting fugetaxis (p<0.0001) in the context of the 0 to 1.2 μM IL-8 gradient.

Further, the differential sensitivities of neutrophil chemotaxis and fugetaxis to wortmannin and 8-Br-cAMP were demonstrated. Both PI3K and cAMP have been shown to play a significant role in gradient sensing and directional decision making in eukaryotic cells including Dictyostelium, neutrophils, neurons and T-cells. It was also demonstrated that intracytoplasmic cAMP levels differentially inhibit fugetaxis or chemorepulsion which is consistent with previous findings in eukaryotic neurons and T-cells. Wortmannin system inhibited the predominant direction of movement observed under control conditions in the gradient and augments the contrary directional decision which was not previously predominantly seen under control conditions. The distribution of PI3K and PTEN to the leading or trailing edge of the cell is thought to play a critical role in directional decision making in the context of eukaryotic cell chemotaxis. Chemotaxis is down-regulated in the context of wortmannin in the shallow 0 to 120 nM gradient as expected but surprisingly fugetaxis is augmented. When fugetaxis is inhibited by wortmannin in the steeper IL-8 gradient chemotaxis is augmented. This data supports previous work indicating a PI3K independent pathway governing the directional decision of neutrophils and that indicates that the leading and trailing edges can be interchangeable and that the localization of PI3K and or a second protein or proteins such as PTEN can determine the directional decision in the absence of PI3K activity.

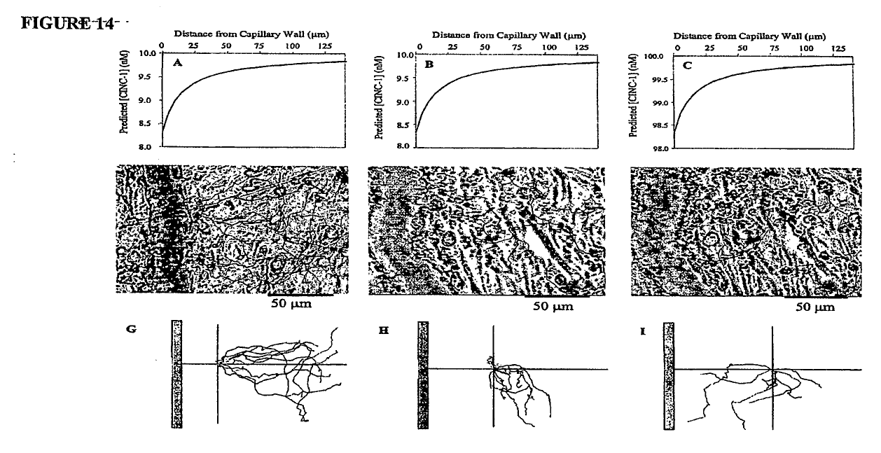

Having demonstrated robust bi-directional migration of neutrophils to a defined IL-8 gradient in vitro, this observation was confirmed in viva. Neutrophil migratory responses to the IL-8 orthologue, cytokine induced neutrophil chemoattractant-1 (CINC-1,) was evaluated in a rat model. CINC-1 and IL-8 are known and potent chemoattractants for murine neutrophils and signal migration via CXCR2. Rat CINC-1, unlike rat IL-8 has been cloned and is commercially available. Diffusive chemokine gradients were established in tissues adjacent to venules in mesentery which has been exteriorized in anesthetized animals. Diffusive gradients with peak concentrations adjacent to the point of superfusion and declining towards the venule as a result of adsorption of chemokine by matrix proteins, binding of chemokine to receptor and internalization of chemokine/receptor complexes and representation of chemokine on the luminal surface of endothelial cells. Chemokine gradients can be mathematically modeled in this context on the basis of predicable absorption and diffusion rates of the chemokine through tissue (FIGS. 14A through C). It is important to note that this gradient model predicts that the gradient shape between the source of chemokine superfusion and vessel wall is the same shape for all peak chemokine concentrations. The steepness of the gradient at any fixed point between the superfused chemokine and the vessel wall will therefore remain constant while the absolute concentration of chemokine seen at that point varies. The in vivo model therefore proves to be of use in determining the effect of gradient steepness and absolute concentration on the directional decision of cells in vivo.

Two types of experiments were established in this model. First, mesenteric tissue adjacent to a venule was superfused with chemokine at a fixed concentration of 1 nM, 10 nM or 100 nM for 90 minutes. Neutrophil migration was subsequently recorded by time lapse video microscopy and migrating neutrophils positively identified as such by subsequent acridine orange staining (FIG. 14D). Under these conditions, peak transendothelial migration of neutrophils from the blood occurred towards peak concentrations of the chemokine of 10 nM. Concentrations of 1 nM lead to minimal neutrophil adhesion to the luminal surface of the venule and transmigration and concentrations of 100 nM lead to accumulation of neutrophils around the vessel without transmigration towards the peak concentrations of CINC-1 (data not shown). In the second set of experiments the application of a chemokine gradients with a peak concentration of 10 nM (FIG. 14E and Video 6) or 100 nM for 45 minutes was replaced sequentially by a gradient with a peak concentration of 100 nM (FIG. 14F and Video 7) or 10 nM in order to replace a potentially chemotactic gradient with a fugetactic gradient. Cell migration was tracked as previous described using MetaMorph software (FIG. 14I).

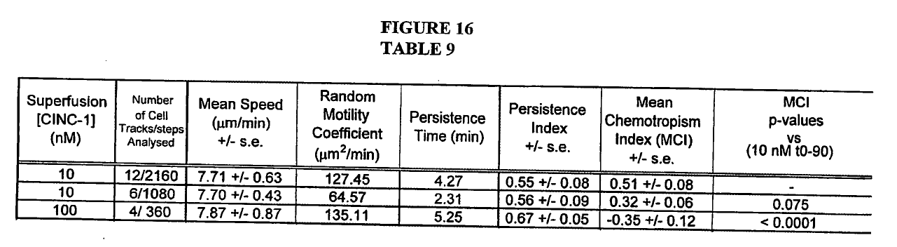

Cells were observed undergoing chemotaxis out of the mesenteric venule towards peak concentrations of chemokine of 10 nM in adjacent tissues as previously described (FIG. 12H). However, in contrast, when a gradient with a peak concentration at the point of superfusion of 100 nM replaced the previous lower concentration of chemokine, neutrophils were observed to migrate back towards the mesenteric venule (FIG. 14I). Directional movement up or down a gradient was quantitated as previously described for cells migrating in defined gradients in vitro. Cell velocities and random motility coefficients of neutrophils migrating under these gradient conditions in vivo towards or away from peak concentrations of chemokine of 10 nM and 100 nM varied between 7.70 and 7.87 μm per minute and 64.57 to 135.11 μm²/min (FIG. 16, Table 9). These velocities and random motility coefficients were not significantly different from those seen for cell migrating in the gradients of similar steepness and absolute concentration of chemokine in vitro and varied between 2.0 and 5.1 microns/minute and between 504.11 and 831.33 μm²/min. Interestingly persistence times for cells migrating in vivo were significantly less in vivo (2.31 to 5.25 min) than those seen in vitro (11.1 to 14.6 min) in gradients with peak concentrations of 10 nM and 100 nM and 12 nM and 120 nM, respectively and may reflect the complexity of the surface over which the cells migrate in vivo as compared to the in vitro setting. Finally quantitative measurement of the directional bias of cells in gradients in vivo, including mean chemotropism index indicated that cells predominantly migrate towards a diffusive gradient of CINC-1 with a peak concentration of 10 nM with MCI of +0.32+/−0.06 whereas cells moved away when this gradient was replaced with a gradient with a peak concentration of 100 nM CINC-1 with a MCI of −0.35+/−0.12.

### CONCLUSIONS

The in vitro and in vivo presented above rigorously demonstrate the ability neutrophils to move up or down a chemokine gradient. In contrast to the current paradigm, which argues that neutrophils only enter tissues as a result of positive chemitocatic agents, these findings indicate the existence of neutrophil chemorepellents which actively exclude neuttrophils form heathy uninfected tissues. Ultimately, these findings raise the possibility for the design of a novel class of anti-inflammatory agents which actively repel neutrophils from specific anatomic sites.

Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A method for identifying a nucleic acid expressed in a concentration dependent manner, comprising:
determining a first nucleic acid expression profile of a first cell at a first position in an agent concentration gradient,
determining a second nucleic acid expression profile of a second cell at a second position in the agent concentration gradient,
determining a difference between the first and second nucleic acid expression profiles,
wherein the first position in the agent concentration gradient corresponds to a first concentration of agent, and the second position in the agent concentration gradient corresponds to a second concentration of agent, and at least the second cell has migrated through the agent concentration gradient.

2. The method of claim 1, wherein the nucleic acid expression profile is an mRNA expression profile.

3. The method of claim 1, wherein the agent concentration gradient is a ligand concentration gradient.

4. The method of claim 1, wherein the agent concentration gradient is a chemokine concentration gradient.

5. The method of claim 4, wherein the chemokine concentration gradient is selected from the group consisting of SDF-1α, SDF-1β, IP-10, MIG, GROα, GROβ, GROγ, IL-8, PF4, MCP, MIP-1α, MIP-1β, MIP-1γ (mouse), MCP-2, MCP-3, MCP-4, MCP-5 (mouse), RANTES, fractalkine, lymphotactin, CXC, IL-8, GCP-2, ENA-78, NAP-2, IP-10, MIG, I-TAC, SDF-1α, BCA-1, PF4, Bolekine, HCC-1, Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), C-10 (mouse), C Lymphotactin, and CX₃C Fracktelkine (Neurotactin) and ITAC concentration gradients.

6. The method of claim 3, wherein the agent concentration gradient is a cytokine concentration gradient.

7. The method of claim 6, wherein the cytokine concentration gradient is selected from the group consisting of PAF, N-formylated peptides, C5a, LTB₄ and LXA₄, CXC, IL-8, GCP-2, GRO, GROα, GROβ, GROγ, ENA-78, NAP-2, IP-10, MIG, I-TAC, SDF-1α, BCA-1, PF4, Bolekine, MIP-1α, MIP-1β, RANTES, HCC-1, MCP-1, MCP-2, MCP-3, MCP-4, MCP-5 (mouse), Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), MIP-1γ (mouse), C-10 (mouse), C Lymphotactin, and CX₃C Fracktelkine (Neurotactin), SDF-1α, SDF-1β, met-SDF-1β, IL-1, IL-2, IL-3, IL-4, IL-5, IL-6, IL-7, IL-10, IL-12, IL-15, IL-18, TNF, IFN-α, IFN-β, IFN-γ, granulocyte-macrophage colony stimulating factor (GM-CSF), granulocyte colony stimulating factor (Q-CSF), macrophage colony stimulating factor (M-CSF), TGF-β, FLT-3 ligand, VEGF, DMDA, endothelin and CD40 ligand concentration gradients.

8. The method of claim 1, wherein the first concentration of agent is a zero concentration of agent, and the second concentration of agent is a non-zero concentration of agent.

9. The method of claim 1, wherein the first concentration of agent is greater than the second concentration of agent.

10. The method of claim 1, wherein the first cell has migrated through the agent concentration gradient.

11. The method of claim 1, wherein migration through the agent concentration gradient is fugetactic migration.

12. The method of claim 1, wherein migration through the agent concentration gradient is chemotactic migration.

13. The method of claim 1, wherein the nucleic acid expression profile is determined by Northern analysis.

14. The method of claim 1, wherein the nucleic acid expression profile is determined by polymerase chain reaction (PCR) analysis.

15. The method of claim 1, wherein the nucleic acid expression profile is determined by nucleic acid chip analysis.

16. The method of claim 1, wherein the gradient is a step gradient.

17. The method of claim 1, wherein the gradient is a continuous gradient.

18. The method of claim 1, wherein the gradient comprises a second gradient co-existing with the first gradient.

19. The method of claim 1, wherein the first and second cells are adult cells.

20. The method of claim 1, wherein the first and second cells are human cells.

21. The method of claim 1, wherein the first and second cells are primary cells.

22. The method of claim 1, wherein the first and second cells are hemopoietic cells.

23. The method of claim 1, wherein the first and second cells are T lymphocytes.

24. The method of claim 1, wherein the first and second cells are neural cells.

25. A method for identifying a compound that can modulate cell migration in one or more agent concentration gradients comprising:
contacting a migratory cell in an agent concentration gradient with a test compound;
determining the nucleic acid expression profile in the cell; and
identifying a change in expression of a gene expression product.

26. The method of claim 25, wherein the cell migration is chemotaxic migration and the gene expression product is a chemotactic specific gene expression product.

27. The method of claim 25, wherein the cell migration is fugetaxic migration and the gene expression product is a chemotactic specific gene expression product.

28. The method of claim 25, wherein the nucleic acid expression profile is an mRNA expression profile.

29. The method of claim 25, wherein the agent concentration gradient is a ligand concentration gradient.

30. The method of claim 25, wherein the agent concentration gradient is a chemokine concentration gradient.

31. The method of claim 30, wherein the chemokine concentration gradient is selected from the group consisting of SDF-1α, SDF-1β, IP-10, MIG, GROα, GROβ, GROγ, IL-8, PF4, MCP, MIP-1α, MIP-1β, MIP-1γ (mouse), MCP-2, MCP-3, MCP-4, MCP-5 (mouse), RANTES, fractalkine, lymphotactin, CXC, IL-8, GCP-2, ENA-78, NAP-2, IP-10, MIG, I-TAC, SDF-1α, BCA-1, PF4, Bolekine, HCC-1, Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), C-10 (mouse), C Lymphotactin, and CX₃C Fracktelkine (Neurotactin) and ITAC concentration gradients.

32. The method of claim 25, wherein the agent concentration gradient is a cytokine concentration gradient.

33. The method of claim 32, wherein the cytokine concentration gradient is selected from the group consisting of the cytokine concentration gradient is selected from the group consisting of PAF, N-formylated peptides, C5a, LTB₄ and LXA₄, CXC, IL-8, GCP-2, GRO, GROα, GROβ, GROγ, ENA-78, NAP-2, IP-10, MIG, I-TAC, SDF-1α, BCA-1, PF4, Bolekine, MIP-1α, MIP-1β, RANTES, HCC-1, MCP-1, MCP-2, MCP-3, MCP-4, MCP-5 (mouse), Leukotactin-1 (HCC-2, MIP-5), Eotaxin, Eotaxin-2 (MPIF2), Eotaxin-3 (TSC), MDC, TARC, SLC (Exodus-2, 6CKine), MIP-3α (LARC, Exodus-1), ELC (MIP-3β), I-309, DC-CK1 (PARC, AMAC-1), TECK, CTAK, MPIF1 (MIP-3), MIP-5 (HCC-2), HCC-4 (NCC-4), MIP-1γ (mouse), C-10 (mouse), C Lymphotactin, and CX₃C Fracktelkine (Neurotactin), SDF-1α, SDF-1β, met-SDF-1β, IL-1, IL-2, IL-3, IL-4, IL-5, IL-6, IL-7, IL-10, IL-12, IL-15, IL-18, TNF, IFN-α, IFN-β, IFN-γ, granulocyte-macrophage colony stimulating factor (GM-CSF), granulocyte colony stimulating factor (G-CSF), macrophage colony stimulating factor (M-CSF), TGF-β, FLT-3 ligand, VEGF, DMDA, endothelin and CD40 ligand concentration gradients.

34. The method of claim 25, wherein the nucleic acid expression profile is determined at two different concentrations of agent.

35. The method of claim 34, wherein the two different concentrations of agent are a zero concentration of agent and a non-zero concentration of agent.

36. The method of claim 35, wherein the cell at a zero concentration of gradient has migrated through a gradient.

37. The method of claim 25, wherein the nucleic acid expression profile is determined by Northern analysis.

38. The method of claim 25, wherein the nucleic acid expression profile is determined by polymerase chain reaction (PCR) analysis.

39. The method of claim 25, wherein the nucleic acid expression profile is determined by nucleic acid chip analysis.

40. The method of claim 25, wherein the gradient is a step gradient.

41. The method of claim 25, wherein the gradient is a continuous gradient.

42. The method of claim 25, wherein the gradient comprises a second gradient co-existing with the first gradient.

43. The method of claim 25, wherein the cell is an adult cell.

44. The method of claim 25, wherein the cell is a human cell.

45. The method of claim 25, wherein the cell is a primary cell.

46. The method of claim 25, wherein the cell is a hemopoietic cell.

47. The method of claim 25, wherein the cell is a T lymphocyte.

48. The method of claim 25, wherein the cell is a neural cell.

49. A method for inhibiting cell fugetaxis comprising
contacting a cell undergoing or likely to undergo fugetaxis with an agent that inhibits a fugetaxis specific gene expression product in an amount effective to inhibit fugetaxis.

50. The method of claim 49, wherein the fugetaxis specific gene expression product is a nucleic acid or a peptide.

51. The method of claim 49, wherein the fugetaxis specific gene expression product is a signaling molecule.

52. The method of claim 49, wherein the signaling molecule is selected from the group consisting of cell division cycle 42, annexin A3, Rap1 guanine nucleotide exchange factor, adenylate cyclase 1, JAK binding protein, and Rho GDP dissociation inhibitor alpha.

53. The method of claim 52, wherein the signaling molecule is selected from the group consisting of cell division cycle 42 (cdc42), ribosomal protein S6 kinase, BAI1-associated protein 2, GTPase regulator associated with FAK, protein kinase C-beta 1, phosphoinositide-specific phospholipase C-beta 1, nitric oxide synthase 1, phosphatidylinositol -4-phosphate 5-kinase, and MAP kinase kinase kinase kinase 4.

54. The method of claim 49, wherein the fugetaxis specific gene expression product is a extracellular matrix related molecule.

55. The method of claim 54, wherein the extracellular matrix related molecule is selected from the group consisting of chitinase 3-like 1 (cartilage glycoprotein-39), carcinoembryonic antigen-related cell adhesion molecule 6, matrix metalloproteinase 8 (neutrophil collagenase), integrin cytoplasmic domain-associated protein 1, ficolin (collagenfibrinogen domain-containing) 1, epithelial V-like antigen 1, vascular endothelial growth factor (VEGF), fibulin 1, carcinoembryonic antigen-related cell adhesion molecule 3, and lysosomal-associated membrane protein 1.

56. The method of claim 49, wherein the fugetaxis specific gene expression product is a cytoskeleton related molecule.

57. The method of claim 56, wherein the cytoskeleton related molecule is selected from the group consisting of ankyrin 1 (erythrocytic), S100 calcium-binding protein A12 (calgranulin C), plectin 1 (intermediate filament binding protein, 500 kD), microtubule-associated protein RPEB3, microtubule-associated protein 1A like protein (MILP), capping protein (actin filament, gelsolin-like), and ankyrin 2 (neuronal).

58. The method of claim 49, wherein the fugetaxis specific gene expression product is a cell cycle molecule.

59. The method of claim 58, wherein the cell cycle molecule is selected from the group consisting of v-kit Hardy-Zuckerman 4 feline sarcoma viral oncogene homolog, lipocalin 2 (oncogene 24p3), lectin, (galactoside-binding, galectin 3), RAB31 (member RAS oncogene family), disabled (Drosophila) homolog 2 (mitogen-responsive phosphoprotein), RAB9 (member RAS oncogene family, pseudogene 1), and growth differentiation factor 8.

60. The method of claim 49, wherein the fugetaxis specific gene expression product is an immune response related molecule.

61. The method of claim 61, wherein the immune response related molecule is selected from the group consisting of major histocompatibility complex (class II, DR alpha), S100 calcium-binding protein A8 (calgranulin A), small inducible cytokine subfamily A (Cys-Cys), eukaryotic translation initiation factor 5A, small inducible cytokine subfamily B (Cys-X-Cys) (member 6, granulocyte chemotactic protein 2), Fc fragment of IgG binding protein, CD24 antigen (small cell lung carcinoma cluster 4 antigen), MHC class II transactivator, T cell receptor (alpha chain), T cell activation (increased late expression), MKP-1 like protein tyrosine phosphatase, T cell receptor gamma constant 2, T cell receptor gamma locus, cytochrome P450 (subfamily IVF, polypeptide 3, leukotriene B4 omega hydroxylase).

62. The method of claim 49, wherein the cell is an immune cell.

63. The method of claim 49, wherein the contacting occurs in vivo in a subject having or at risk of having an abnormal immune response.

64. The method of claim 49, wherein the cell is a neural cell.

65. The method of claim 49, wherein the fugetaxis specific gene expression product is chemokine (C-X3-C) receptor 1.

66. A method for inhibiting cell chemotaxis comprising
contacting a cell undergoing or likely to undergo chemotaxis with an agent that inhibits a chemotaxis specific gene expression product in an amount effective to inhibit chemotaxis.

67. The method of claim 66, wherein the chemotaxis specific gene expression product is a nucleic acid or a peptide.

68. The method of claim 66, wherein the cell is a immune cell.

69. The method of claim 66, wherein the contacting occurs in vivo in a subject having or at risk of having an abnormal immune response.

70. The method of claim 66, wherein the abnormal immune response is an inflammatory response.

71. The method of claim 66, wherein the abnormal immune response is an autoimmune response.

72. The method of claim 71, wherein the autoimmune response is selected from the group consisting of rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

73. The method of claim 66, wherein the abnormal immune response is a graft versus host response.

74. The method of claim 66, wherein the chemotaxis specific gene expression product is a signaling molecule.

75. The method of claim 74, wherein the signaling molecule is selected from the group consisting of G protein-coupled receptor kinase 6, vaccinia related kinase 1, PTK2 protein tyrosine kinase 2, STAM-like protein containing SH3 and ITAM domains 2, signal-induced proliferation-associated gene 1, CD47 antigen (Rh-related antigen, integrin-associated signal transducer), and protein tyrosine phosphatase (non-receptor type 12).

76. The method of claim 74, wherein the signaling molecule is selected from the group consisting of PTK2 (focal adhesion kinase), MAP kinase kinase kinase kinase 2, guanine nucleotide binding protein, PT phosphatase (receptor), cdc42-binding protein kinase beta, Ral GEF (RalGPS1A), MAP kinase 7, autotaxin, inositol 1,4,5-triphosphate receptor, phosphoinositide-3-kinase gamma, PT phosphatase (non-receptor), RAS p21 protein activator (GAP), RAS guanyl releasing protein 2, and Arp23 complex 20 kDa subunit.

77. The method of claim 66, wherein the chemotaxis specific gene expression product is a extracellular matrix related molecule.

78. The method of claim 77, wherein the extracellular matrix related molecule is selected from the group consisting of spondin 1 (f-spondin, extracellular matrix protein), collagen type XVIII (alpha 1), CD31 adhesion molecule, tetraspan 3, glycoprotein A33, and angio-associated migratory cell protein.

79. The method of claim 66, wherein the chemotaxis specific gene expression product is a cytoskeleton related molecule.

80. The method of claim 79, wherein the cytoskeleton related molecule is selected from the group consisting of actin related protein 23 complex (subunit 4, 20 kD), tropomyosin 2 (beta), SWISNF related matrix associated actin dependent regulator of chromatin (subfamily a, member 5), spectrin beta (non-erythrocytic 1), myosin light polypeptide 5 (regulatory), keratin 1, plakophilin 4, and capping protein (actin filament, muscle Z-line, alpha 2).

81. The method of claim 66, wherein the chemotaxis specific gene expression product is a cell cycle molecule.

82. The method of claim 81, wherein the cell cycle molecule is selected from the group consisting of FGF receptor activating protein 1, v-maf musculoaponeurotic fibrosarcoma (avian) oncogene homolog, cyclin-dependent kinase (CDC2-like) 10, TGFB inducible early growth response 2, retinoic acid receptor alpha, anaphase promoting complex subunit 10, RAS p21 protein activator (GTPase activating protein, 3-Ins-1,3,4,5, -P4 binding protein), cell division cycle 27, programmed cell death 2, c-myc binding protein, mitogen-activated protein kinase kinase kinase 1, TGF beta receptor III (betaglycan, 300 kDa), and G1 to S phase transition 1.

83. The method of claim 66, wherein the chemotaxis specific gene expression product is an immune response related molecule.

84. The method of claim 83, wherein the immune response related molecule is selected from the group consisting of major histocompatibility complex class II DQ beta 1, bone marrow stromal cell antigen 2, Burkitt lymphom a receptor 1 (GTP binding protein, CXCR5), CD7 antigen (p41), Stat2 type a, T cell immune regulator 1, and interleukin 21 receptor.

85. The method of claim 66, wherein the cell is a neural cell.

86. A method for promoting cell fugetaxis comprising
contacting a cell with a non-chemokine agent that promotes fugetaxis in an amount effective to promote fugetaxis.

87. The method of claim 86, wherein the contacting occurs in vivo in a subject having a disorder characterized by lack of fugetaxis.

88. The method of claim 86, wherein the cell is a hematopoietic cell.

89. The method of claim 88, wherein the hematopoietic cell is a T lymphocyte.

90. The method of claim 86, wherein the cell is a neural cell.

91. A method for promoting cell chemotaxis comprising
contacting a cell with a non-chemokine agent that promotes chemotaxis in an amount effective to promote chemotaxis.

92. The method of claim 91, wherein the contacting occurs in vivo in a subject having a disorder characterized by lack of chemotaxis.

93. The method of claim 91, wherein the cell is a hematopoietic cell.

94. The method of claim 93, wherein the hematopoietic cell is a T lymphocyte.

95. The method of claim 91, wherein the cell is a neural cell.

96. The method of claim 62, wherein the immune cell is a selected from the group consisting of T-cells, B-cells, NK cells, dendritic cells, monocytes and macrophages.

97. The method of claim 96, wherein the immune cell is an inflammatory selected from the group consisting of neutrophils, basophils, eosinophils and mast cells.

98. The method of claim 88, wherein the hematopoietic cell is an immune cell is a selected from the group consisting of T-cells, B-cells, NK cells, dendritic cells, monocytes and macrophages.

99. The method of claim 98, wherein the immune cell is an inflammatory selected from the group consisting of neutrophils, basophils, eosinophils and mast cells.

100. A method for promoting neutrophil chemotaxis comprising contacting a neutrophil with IL-8 in an amount effective to promote chemotaxis.

101. The method of claim 100, wherein the neutrophil is contacted with a low concentration of IL-8.

102. The method of claim 101, wherein the low concentration of IL-8 is between about 10 ng/ml to about 500 ng/ml.

103. The method of claim 100, wherein the contacting occurs in vivo in a subject having a disorder characterized by lack of neutrophil chemotaxis.

104. The method of claim 103, wherein the disorder is selected from the group consisting of bacterial infections and granulomatous diseases.

105. The method of claim 103, wherein IL-8 is provided to the subject on a material surface coated with IL-8.

106. The method of claim 105, wherein the material surface is implanted within the subject.

107. The method of claim 103, wherein IL-8 is provided to the subject in a controlled release formulation.

108. A method for promoting neutrophil fugetaxis comprising contacting a neutrophil with IL-8 in an amount effective to promote fugetaxis.

109. The method of claim 108, wherein the neutrophil is contacted with a high concentration of IL-8.

110. The method of claim 109, wherein the concentration of IL-8 is between about 1 microgram/ml to about 10 micrograms/ml.

111. The method of claim 108, wherein the contacting occurs in vivo in a subject having a disorder characterized by lack of neutrophil fugetaxis.

112. The method of claim 111, wherein the disorder is selected from the group consisting of inflammatory or immune mediated diseases, rejection of a transplanted organ or tissue, rheumatoid arthritis, automimmune diseases and asthma.

113. The method of claim 108, wherein IL-8 is provided to the subject on a material surface coated with IL-8.

114. The method of claim 113, wherein the material surface is implanted within the subject.

115. The method of claim 108, wherein IL-8 is provided to the subject in a controlled release formulation.

116. A method for inhibiting IL-8 induced neutrophil chemotaxis comprising contacting a neutrophil with wortmannin in an amount effective to inhibit chemotaxis and optionally induce fugetaxis by the neutrophil.

117. A method for inhibiting IL-8 induced neutrophil fugetaxis comprising contacting a neutrophil with LY294002 in an amount effective to inhibit fugetaxis and optionally induce chemotaxis by the neutrophil.

118. The method of claim 4, wherein the chemokine concentration gradient is a SDF-1 concentration gradient.

119. The method of claim 4, wherein the chemokine concentration gradient is an IL-8 concentration gradient.

120. The method of claim 6, wherein the cytokine concentration gradient is a SDF-1 concentration gradient.

121. The method of claim 6, wherein the cytokine concentration gradient is an IL-8 concentration gradient.
